(19) United States
(12) Patent Application Publication
(10) Pub. No.: US 2016/0338361 A1
(43) Pub. Date: Nov. 24, 2016

(54) SYSTEMS, METHODS, AND COMPOSITIONS FOR PROMOTING PATHOGEN CONTROL AND FOOD PRESERVATION

(71) Applicant: MICRO-NATURE LLC, San Francisco, CA (US)

(72) Inventors: Richard Baird SMITTLE, Morgantown, WV (US); Gregory Dean SUNVOLD, Lewisburg, OH (US); John Boyd PHELPS, Andover, NJ (US); John HOMMEYER, San Francisco, CA (US)

(73) Assignee: MICRO-NATURE LLC, San Francisco, CA (US)

(21) Appl. No.: 15/116,511

(22) PCT Filed: Feb. 4, 2015
(86) PCT No.: PCT/US2015/014525

§ 371 Date: Aug. 3, 2016

Related U.S. Application Data
(60) Provisional application No. 61/935,408, filed on Feb. 4, 2014.
(60) Provisional application No. 62/027,222, filed on Jul. 21, 2014.

(51) Int. Cl. A01N 63/02; A23K 10/16; A23L 3/3571; A23K 50/48; A23L 33/135; C12N 1/20; A23K 40/30

(52) U.S. Cl. CPC A01N 63/02; C12N 1/20; A23K 10/16; A23K 40/30; A23K 50/48; A23L 33/135; A23L 3/3571; A23V 2002/00

(57) ABSTRACT

The present teachings disclose a topical application composition. The topical application composition includes: (1) a non-fermenting bacteria that is in a substantially non-fermenting state and is produced from fermentation of the bacteria; (2) a fermentation byproduct produced from the fermentation; and (3) a fluid portion.

### RELATED CASES

This application claims priority to U.S. provisional applications No. 61/935,408, filed Feb. 4, 2014, and No. 62/027,222, filed Jul. 21, 2014, and are incorporated by reference for all purposes.

### FIELD

The present teachings relate generally to systems, methods, and compositions used to promote food preservation and safety and to sanitize inedible surfaces. More specifically, the present teachings relate to systems, methods, and compositions used to treat the surface of animal and human food products, as well inedible surfaces, with a topical application that promotes food safety and preservation, and that decontaminates inedible surfaces, in a safe and effective manner.

### BACKGROUND

Human and pet foods are currently made by a number of processing techniques. These techniques typically require deactivating pathogenic and food-spoilage microorganisms associated with the ingredients, equipment, and/or conditions used to process food. For example, certain foods are harvested close to soil, significantly increasing the chances of contamination with food-spoilage microorganisms, or pathogens such as E. coli or Salmonella. Likewise, meat used in pet food are often byproducts of rendering, which typically produces food contaminated with Salmonella. In fact, contaminants such as pathogens and food-spoilage microorganisms may arise from almost anywhere, including from airborne ingredients and dust particles, from latent pathogens or food-spoilage microorganisms associated with equipment surfaces, from externally applied ingredients, and the like.

One common human food (such as canned vegetables, soups and meats) and pet food processing technique used to decontaminate foods is retorting or autoclaving. The retort process, however, is problematic in that it requires high amounts of heat, lengthy exposure of food to heat, and in some cases high amounts of pressure. The significant amount of heating, pressure and time is not only costly, but also alters the nutrient quality of food in an undesirable manner. As such, improved systems, methods and compositions relating to food manufacture that overcome the drawbacks of the retort process and yet still lessen the risk of product re-contamination are desirable.

Moreover, given the daunting effort to keep food free from contamination by pathogens and/or spoilage microorganisms, there remains a need for food processing techniques that effectively produce pathogen-free and shelf-stable food in a safe, effective, and inexpensive manner that does not compromise the quality of the food. What is therefore needed are systems, methods, and compositions that can be used to treat the surface of food products and inedible surfaces to produce food products that are safe and shelf-stable and inedible surfaces that are decontaminated.

### SUMMARY OF THE INVENTION

In one aspect, the present teachings disclose a topical application composition. The composition includes (i) a non-fermenting bacteria that is in a substantially non-fermenting state and is produced from fermentation of the bacteria; (ii) a fermentation byproduct produced from fermentation; and (iii) a fluid portion. The fluid portion may includes at least one member chosen from a group comprising water, growth medium, culture energy source, and buffered solution. According to one embodiment of the present teachings, the fluid portion is less than about 1% by weight of the topical application composition.

According to one embodiment of the present teachings, the topical application the non-fermenting bacteria and the fermentation byproduct constitute a solid residue that is present in the topical application composition in an original amount, and the topical application composition includes the solid residue in a concentrated amount that is between about 2 times and about 20 times greater than said original amount. Preferably, the concentrated amount is about 8 times greater than said original amount.

According to one embodiment of the present teachings, an amount of non-fermenting bacteria in the topical application is between about 1×10³ cfu/(gram of the topical application) and about 1×10¹⁰ cfu/(gram of the topical application). At least one of the non-fermenting bacteria includes at least one food-safety bacteria or food-preserving bacteria that is at least one member chosen from a group comprising Pediococcus acidilactici, Pediococcus pentosaceus, Lactococcus lactis, Lactococcus cremoris, Lactobacillus delbruckii var bulgaricus, Lactobacillus plantarum, Lactobacillus pentosum, Streptococcus thermophilus, Lactobacillus sakei and Lactobacillus curvatus, Lactobacillus acidophilus, Lactobacillus reuteri, Lactobacillus plantarum, Lactobacillus casei, Lactobacillus johnsonii, Lactobacillus rhamnosus, Lactobacillus gasseri, Bifidobacterium lactis, Bifidobacterium infantis, Bifidobacterium longum, Saccharomyces boulardii, Lactobacillus salivarus, Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescents, Bifidobacterium animalis, Bifidobacterium thermophilium, Enterococcus faecalis, Streptococcus cremoris, Streptococcus salivarius, Streptococcus diacetylactis, Streptococcus intermedius, Lactobacillus paracasei, Streptococcus thermophiles, Streptococcus salivarius subsp. thermophilus, Bacillus cereus, Propionibacterium freundenreichii, Oxalobacter formagenes, Bifidobacterium bifidus, and Saccharomyces cerevisiae. According to another embodiment of the present teachings, at least one of the non-fermenting bacteria includes a health-promoting bacteria that is at least one member chosen from a group comprising Lactobacillus acidophilus, Lactobacillus reuteri, Lactobacillus plantarum, Lactobacillus casei, Lactobacillus johnsonii, Lactobacillus rahamnosus, Lactobacillus gasseri, Bifidobacterium lactis, Bifidobacterium infantis, Bifidobacterium longum, Saccharomyces boulardii, Lactobacillus salivarus, Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescsents, Bifidobacterium animalis, Bifidobacterium thermophilium, Enterococcusfaecalis, Streptococcus cremoris, Streptococcus salivarius, Streptococcus diacetylactis, Streptococcus intermedius, Lactobacillus paracasei, Streptococcus thermophiles, Streptococcus salivarius subsp. Thermophilus, Bacillus cereus, Proprionibacteria freundenreichii, Bacillus coagulans (L. sporegenes), Oxalobacter formagenes, Bifidobacterium bifidus, and Saccharomyces cerevisiae.

A fermentation byproduct may include at least one member chosen from a group comprising bacteriocin, pediocin, hydrogen peroxide, and lactate. The fermentation byproduct may include at least one antimicrobial lactic acid producing bacteria metabolite chosen from a group comprising phenyllactic acid, 3-hydroxyphenyllactic acid, 4-hydroxyphenylactic acid, 3-hydroxy propanaldehyde, 1,2 propandiol, 1,3 propandiol, hydrogen peroxide, ethanol, acetic acid, carbon dioxide, carbonic acid, propanoic acid, butyric acid, cyclic dipeptides, cyclo(L-Phe-L-Pro), cyclo(L P-Traps-4-OH-L-Pro), 3-(R)-hydroxydecanoic acid, 3-hydroxy-5-cic dodecanoic acid, 3-(R)-hydroxy dodecanoic acid, and 3-(R)-hyroxytetradecanoic acid. According to yet another embodiment of the present teachings, the fermentation byproduct includes at least one bacteriocin that is a lantibiotic (Class II) and/or a non-lantibiotic (Class II). According to another embodiment of the present teachings, the fermentation byproduct includes at least one bacteriocin selected from a group comprising nisin A, nisin Z, nisin Q, nisin F, nisin U, nisin U2, salivarcin X, lacticin J46, lacticin 481, lacticin 3147, salivarcin A, salivarcin A2, salivarcin A3, salivarcin A4, salivarcin A5, salivarcin B, streptin, salivaricin A1, streptin, streptococcin A-FF22, BHT-Aa, BHT Ab, mutacin BNY266, mutacin 1140, mutacin K8, mutacin II, smbAB, bovicin HJ50, bovicin HC5, macedocin, plantaricin W, lactocin 5, cyctolysin, enterocin A, divercin V41, divercin M35, bavaricin, coagulin, pediocin PA-1, mundticin, piscicocin CS526, piscicocin 126/Via, sakacin P, leucocin C, sakacin 5X, enterocin CRL35/mundticin, avicin A, mundticin I, enterocin HF, bavaricin A, ubericin A, leucocin A, mesentericin Y105, sakacin G, plantaricin 423, plantaricin C19, curvacin A/sakacin A, carnobacteriocin BM1, enterocin P, piscicoin V1b, penocin A, bacteriocin 31, bacteriocin RC714, hiracin JM79, bacteriocin T8, enterocin SE-K4, carnobacteriocin B2, SRCAM 1580, and CONCENSUS.

In another aspect, the present teachings disclose a substantially pathogen-free and/or spoilage-microorganism-free food composition. The substantially pathogen-free and/or spoilage-microorganism-free food composition includes: (i) a topical application including: (a) a non-fermenting bacteria that is in a substantially non-fermenting state and is produced from fermentation of the bacteria; and (b) a fermentation byproduct produced from the fermentation; and (ii) a food product having a surface that includes the topical application. The substantially pathogen-free and/or spoilage-microorganism-free food composition may also include at least one member chosen from a group comprising flavor enhancer, palatant, stabilizing agent, food coating stabilizer, fragrance, binder, color, and coloring agent. Preferably, the substantially pathogen-free and/or spoilage-microorganism-free food composition includes less than about 10 cfu of a pathogen and/or a food-spoilage microorganism per gram of the food product.

In yet another aspect, the present teachings disclose a method for producing a topical application. The method for producing a topical application includes: (i) mixing a bacteria in a growth culture including a growth medium and an energy source; and (ii) fermenting the bacteria in the presence of the growth culture to produce a fermented growth culture comprising a non-fermenting bacteria and a fermentation byproduct, such that non-fermenting bacteria is in a substantially non-fermenting state. According to one embodiment of the present teachings, in mixing, the growth culture includes a fluid portion. Fermenting may be carried out at a temperature that is between about 28° C. and about 55° C.

According to one embodiment of the present teachings, inoculating includes inoculating the food product with a health-promoting bacteria. The method for producing a topical application may further include concentrating the fermented growth culture by removing a certain amount of the fluid portion from the fermented growth culture. Concentrating may include separating an amount of the fluid portion from the fermented growth culture using at least one technique chosen from a group comprising sedimenting, centrifuging, vacuuming, decanting, drying, freeze drying, spray drying, and evaporating. The method for producing a topical application may further include drying the fermented growth culture.

In yet another aspect, the present teachings disclose a method for producing a safe and/or shelf-stable food product. The method includes: (i) obtaining a topical application and a food product, and the topical application includes a non-fermenting bacteria and a fermentation byproduct, and the non-fermenting bacteria is in a substantially non-fermenting state; (ii) applying the topical application to a surface of the food product and producing an inoculated food product; and (iii) incubating the inoculated food product to produce a shelf-stable food product that is substantially free of pathogens and/or spoilage microorganisms. According to one embodiment of the present teachings, obtaining may include fermenting a bacteria to produce the non-fermenting bacteria and the fermentation byproduct. Preferably, the shelf-stable food product includes less than about 10 cfu of pathogens and/or spoilage microorganisms per gram of shelf-stable food product.

Incubating may be carried out at a temperature that is between about 28° C. and about 55° C. Applying may include applying the topical application to the surface of the food product at a concentration that is between about 0.0001% by weight/weight of said topical application. Applying may be carried out using at least one technique chosen from a group comprising coating, spraying, soaking, misting, aerosolizing, affixing, and atomizing.

According to one embodiment of the present teachings, the food product includes at least one member chosen from a group comprising kibbled food, kibble, expanded food, pelleted food, extruded food, refrigerated food, refrigerated treat, frozen food, frozen treat, biscuit, raw food, fried foods, treat, soft-moist food, soft-moist treat, pellet, fine, broken piece, jerky-style treat, injection-molded treat, treat, supplement, prepared salad ingredient, ground fruit, grounded vegetable, prepared meal, meat, slaughtered carcass, prepared food, meat piece, meat chunk, fabricated meat chunk, fabricated protein chunk, livestock feed, steam-flaked feed, and aquaculture feed. Preferably, the food product has a moisture content of less than about 10% by weight.

The process of producing a safe and/or shelf-stable food product may include packaging the shelf-stable food product. Preferably, the non-fermenting bacteria promotes human or animal health after the shelf-stable food product is consumed by a human or an animal.

In yet another aspect, the present teachings disclose a process for decontaminating an inedible surface. The process includes applying a topical application to an inedible surface such that the topical application substantially kills and/or inhibits growth of pathogens and/or food-spoilage microorganisms on the inedible surface, and wherein the topical application includes a non-fermenting bacteria and a fermentation byproduct, and the non-fermenting bacteria is in a substantially non-fermenting state. Applying may be carried out using at least one technique chosen from a group comprising spraying, misting, washing, soaking, misting, aerosolizing, affixing, and atomizing. According to one embodiment of the present teachings, the inedible surface includes at least one member chosen from a group comprising pipe, tool, chopper, grinder, hammer mill, roller mill, flaker, emulsifier, blender, block pre-breaker, block breaker, extruder, coating equipment, APEC coater, spray bar, dryer, conveyor, pellet mill, steam flaker, vortex mill, storage bin, band saw, knife, cutting surface, countertop, wood chopping block used in food preparation, stainless steel counter top, counter top, bathroom, wet bar, alcohol serving establishment, drainage system, disposal system, sink drain, kitchen sink, toilet, toilet bowl rim, bath drain, bath tub, garbage can, barn environment, barn stall, horse stall, livestock exhibition hall, livestock bedding area, retention pond, sewage holding tank, areas around sewage holding tanks, dog kennel, dog cage, cat cage, cat carrier, dog carrier, cattery, automotive garage, air recirculation system on jet airliner, shrimp shell after meat has been removed, fish parts after fillets have been removed, animal parts after meat has been removed, human hair, dog hair coat, diaper, cream, skin, dermatitis, psoriasis, eczema, bed sore, dentifrice, oral rinse, vaginal rinse, douche, tampon, feminine pad, waste pail, garbage can, dumpster, waste handling container, commercial waste management vehicle, garbage truck, waste hauling equipment, waste capture equipment, bin, can, vehicle, tote, conveyer, waste processing equipment, waste, under-arm, vagina, foot, outer ear, and diaper.

In yet another aspect, the present teachings disclose a wet food composition. The wet food composition includes: (i) a wet food; (ii) a bacteria; (iii) an energy source; and (iv) a buffer; and wherein the wet food composition has a moisture content that is at least about 15% by weight and a pH that is between about 4.5 and about 4.9, and wherein the bacteria is a food-safety bacteria and/or a food-preserving bacteria that is substantially non-fermenting/viable, and wherein the wet food composition is substantially free of pathogens and/or food-spoilage microorganisms. The buffer may be at least one member chosen from a group comprising calcium carbonate, sodium bicarbonate, carbonic acid, pyrophosphates, sodium acid pyrophosphate, malic acid, potassium citrate, sodium citrate, calcium citrate, monopotassium phosphate, potassium tartrate, vinegar and tricalcium phosphate.

According to one embodiment of the present teachings, the wet food composition further includes a salt and/or a syneresis-controlling substance. A syneresis-controlling substance may be at least one member chosen from a group comprising pea powder, gum arabic, guar gum, hydrocolloid, carboxymethylcellulose, locust bean gum, cassia gum, carageenan, iota-carageenan, kappa-carageenan, milk, milk product, milk protein, casein, pork plasma, textured vegetable protein, gluten, corn gluten, wheat gluten, starch, corn starch, rice starch, potato starch, tapioca starch, sorghum starch, oat starch, soy, soy protein, soy protein concentrate, soy protein isolate, egg, egg derivatives, transglutaminase, gelatin, and polysaccharide. A salt may be at least one member chosen from a group comprising sodium chloride, potassium chloride, sea salt, and calcium chloride.

A concentration of food-safety bacteria and/or food-preserving bacteria in the wet food composition may be between about 1×10³ cfu/g of wet food composition and about 1×10⁹ cfu/g of wet food composition. According to one embodiment of the present teachings, a food-safety bacteria and/or a food-preserving bacteria is at least one member chosen from a group comprising Pediococcus acidilactici, Pediococcus pentosaceus, Lactococcus lactis, Lactococcus cremoris, Lactobacillus delbruckii var bulgaricus, Lactobacillus plantarum, Lactobacillus pentosum, Streptococcus thermophilus, Lactobacillus sakei and Lactobacillus curvatus, Lactobacillus acidophilus, Lactobacillus reuteri, Lactobacillus plantarum, Lactobacillus casei, Lactobacillus johnsonii, Lactobacillus rhamnosus, Lactobacillus gasseri, Bifidobacterium lactis, Bifidobacterium infantis, Bifidobacterium longum, Saccharomyces boulardii, Lactobacillus salivarus, Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescents, Bifidobacterium animalis, Bifidobacterium thermophilium, Enterococcus faecalis, Streptococcus cremoris, Streptococcus salivarius, Streptococcus diacetylactis, Streptococcus intermedius, Lactobacillus paracasei, Streptococcus thermophiles, Streptococcus salivarius subsp. thermophilus, Bacillus cereus, Propionibacterium freundenreichii, Oxalobacter formagenes. The wet food composition may also include a health-promoting bacteria that is at least one member chosen from a group comprising Lactobacillus acidophilus, Lactobacillus reuteri, Lactobacillus plantarum, Lactobacillus casei, Lactobacillus johnsonii, Lactobacillus rahamnosus, Lactobacillus gasseri, Bifidobacterium lactis, Bifidobacterium infantis, Bifidobacterium longum, Saccharomyces boulardii, Lactobacillus salivarus, Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescsents, Bifidobacterium animalis, Bifidobacterium thermophilium, Enterococcus faecalis, Streptococcus cremoris, Streptococcus salivarius, Streptococcus diacetylactis, Streptococcus intermedius, Lactobacillus paracasei, Streptococcus thermophiles, Streptococcus salivarius subsp. Thermophilus, Bacillus cereus, Proprionibacteria freundenreichii, Bacillus coagulans (L. sporegenes), and Oxalobacter formagenes.

Preferably, the wet food composition is shelf-stable for a time that at least about six months.

In yet another aspect, the present teachings disclose a process for producing a wet food composition. The process includes (i) obtaining one or more food ingredients, an energy source, a food-safety bacteria and/or food-preserving bacteria, and a buffer; (ii) mixing one or more of food ingredients to produce a food product; (iii) inoculating the food product with the food-safety bacteria and/or the food-preserving bacteria to produce an inoculated food product; (iv) incubating the inoculated food product to produce an incubated food product, wherein the incubating is sufficient to produce a pH in the incubated food product that is less than about 4.3; and (v) adding the buffer to the incubated food product to produce a wet food composition having a pH that is between about 4.5 and about 4.9; wherein the wet food composition has a moisture content that is at least about 15% by weight, and the wet food composition is substantially free of pathogens and/or spoilage microorganisms. According to another embodiment of the present teachings, inoculating includes inoculating the food product with a health-promoting bacteria. According to another embodiment of the present teachings, the process for producing a wet food composition further includes adding a salt and/or a syneresis-controlling substance to the wet food composition.

### BRIEF DESCRIPTION OF THE DRAWINGS

FIG. 1 is a flowchart showing certain salient steps used in a process for producing a topical application, according to one embodiment of the present teachings.

FIG. 2 is a flowchart, showing certain salient steps that involve using a topical application used in a process for producing a food product that is substantially free of pathogens and/or food-spoilage microorganisms, according to one embodiment of the present teachings.

FIG. 3 is a flowchart showing certain salient steps used in a process for producing a wet food composition, according to one embodiment of the present teachings.

FIG. 4 is a graph showing death of salmonella surrogates on fruit and vegetables that are coated with fermented salmon meal, and the salmonella death is presented on a logarithmic scale versus time.

FIG. 5 is a graph showing death of salmonella surrogates on fruit and vegetables that are coated with digested salmon meal, and the salmonella death is presented on a logarithmic scale versus time.

FIG. 6 is a graph showing death of salmonella surrogates on fruit and vegetables that are coated with fermented beef broth, and the salmonella death is presented on a logarithmic scale versus time.

FIG. 7 is a graph showing death of salmonella surrogates on fruit and vegetables without treatment, and the salmonella death is presented on a logarithmic scale versus time.

FIG. 8 is a graph showing the impact of Pediococci strains on the death of salmonella surrogates, which are coated in in chicken broth and 2% dextrose, and the salmonella death is presented on a logarithmic scale versus time.

FIG. 9 a graph showing impact of 3% Pediococci fermented beef broth culture on death of salmonella surrogates applied to kibbles, and the salmonella death is presented on a logarithmic scale versus time.

FIG. 10 is a graph showing impact of 3% Pediococci fermented chicken broth culture on death of salmonella surrogates applied to kibbles, and the salmonella death is presented on a logarithmic scale versus time.

FIG. 11 is a graph showing impact of 3% Pediococci, Lactobacilli, Bifidobacterium, and Bacilli fermented beef broth culture on death of salmonella surrogates applied to kibbles, and the salmonella death is presented on a logarithmic scale versus time.

FIG. 12 is a graph showing impact of 3% Pediococci, Lactobacilli, Bifidobacterium, and Bacilli fermented chicken broth culture on death of salmonella surrogates applied to kibbles, and the salmonella death is presented on a logarithmic scale versus time.

FIG. 13 is a graph showing death of salmonella surrogates on kibbles that are not treated with a fermented culture, and the salmonella death is presented on a logarithmic scale versus time.

FIG. 14 is a flowchart showing certain steps used to generate broth sources used in Example 3, below.

FIG. 15 is a bar graph showing the effect of fermentation cultures that were derived through various growth conditions on death of salmonella surrogates that were incubated under various temperatures and time conditions, and the salmonella death is presented on a logarithmic scale versus time.

FIG. 16 is a graph comparing impact of varying concentrations of untreated and treated fermentation cultures on the death of salmonella surrogates, and the salmonella death is presented on a logarithmic scale versus the ratio of a final concentration of Pediococci strains to an original amount of Pediococci strains.

FIG. 17 is a graph comparing varying concentrations of untreated fermentation cultures, including Pediococci strains, stored at varying temperature and time conditions and their effects on inactivating Salmonella surrogates, and inactivation of Salmonella surrogates is presented on a logarithmic scale versus ratio of a final concentration of Pediococci strains to an original amount of Pediococci strains.

FIG. 18 is a graph comparing varying concentrations of untreated and treated fermentation cultures stored at 24° C. for 24 hours and for 48 hours on inactivating Salmonella surrogates, and inactivation of Salmonella surrogates is presented on a logarithmic scale versus ratio of a final concentration of Pediococci strains to an original amount of Pediococci strains.

FIG. 19 is a graph comparing varying concentrations of treated fermentation cultures stored at different temperature on inactivating Salmonella surrogates, and inactivation of Salmonella surrogates is presented on a logarithmic scale versus ratio of a final concentration of Pediococci strains to an original amount of Pediococci strains.

FIG. 20 is a graph showing impact of 3% by weight of Pediococci in fermented chicken broth culture on death of Salmonella surrogates applied to coated kibbles when stored at 24° C. for 24 hours, and the graph also shows time versus the death of Salmonella surrogates presented on a logarithmic scale.

FIG. 21 is a graph showing impact of 3% by weight of Pediococci in fermented chicken broth culture on death of Salmonella surrogates applied to coated kibbles when stored at 24° C. for 48 hours, and the graph also shows time versus the death of Salmonella surrogates presented on a logarithmic scale.

FIG. 22 is a graph showing impact of 3% by weight of Pediococci in fermented chicken broth culture on death of Salmonella surrogates applied to coated kibbles when stored at 37° C. for 24 hours, and the graph also shows time versus the death of Salmonella surrogates presented on a logarithmic scale.

FIG. 23 is a line graph showing impact of 3% by weight of Pediococci in fermented chicken broth culture on the death of Salmonella surrogates applied to coated kibbles when stored at 37° C. for 48 hours, and the graph also shows time versus the death of Salmonella surrogates presented on a logarithmic scale.

FIG. 24 is a graph showing impact of 3% by weight of Pediococci in fermented chicken broth culture on death of Salmonella surrogates applied to coated Kibbles when stored at 37° C. for 4 hours followed by storage at 24° C., and the graph also shows time versus the death of Salmonella surrogates presented on a logarithmic scale.

FIG. 25 is a graph showing death of Salmonella and E. coli O157:H7 surrogates, cat kibbles coated a liquid palatant in combination with resuspended cells, and death of Salmonella and E. coli O157:H7 surrogates is presented on a logarithmic scale versus days of storage at 22° C.

FIG. 26 is a graph showing death of salmonella surrogates on dog kibbles coated with a liquid palatant in combination with resuspended cells, and death of Salmonella surrogates is presented on a logarithmic scale versus days of storage at 22° C.

### DETAILED DESCRIPTION OF THE PREFERRED EMBODIMENTS

In one aspect, the systems, methods, and compositions of the present teachings disclose a topical application composition. As explained further herein, a topical application composition may be applied to a human or animal food surface to promote food preservation and/or food safety. A topical application composition may also be applied to an inedible surface to promote control the growth of pathogens and/or food-spoilage microorganisms on that inedible surface.

According to one embodiment of the present teachings, a topical application composition includes a solid residue and a fluid portion. The solid residue includes bacteria that are in a substantially non-fermenting state and one or more fermentation byproducts. The fluid portion may include any fluid such as a growth media, water or a buffer solution.

In certain embodiments of the present teachings, these bacteria are food-preserving bacteria that promote preservation and shelf-stability of a food on which a topical application is applied by reducing and/or eliminating the growth and/or survival of one or more food-spoilage microorganisms. In other embodiments of the present teachings, these bacteria are food-safety bacteria that promote safety of a food on which a topical application is applied by reducing and/or eliminating the growth and/or survival of one or more pathogens. In certain embodiments of the present teachings, a food-preserving bacteria is a food-safety bacteria.

Representative food-preserving bacteria and/or food-safety bacteria in a topical application may include at least one member chosen from a group comprising Pediococcus acidilactici, Pediococcus pentosaceus, Lactococcus lactis, Lactococcus cremoris, Lactobacillus delbruckii var bulgaricus, Lactobacillus plantarum, Lactobacillus pentosum, Streptococcus thermophilus, Lactobacillus sakei and Lactobacillus curvatus, Lactobacillus acidophilus, Lactobacillus reuteri, Lactobacillus plantarum, Lactobacillus casei, Lactobacillus johnsonii, Lactobacillus rhamnosus, Lactobacillus gasseri, Bifidobacterium lactis, Bifidobacterium infantis, Bifidobacterium longum, Saccharomyces boulardii, Lactobacillus salivarus, Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescents, Bifidobacterium animalis, Bifidobacterium thermophilium, Enterococcus faecalis, Streptococcus cremoris, Streptococcus salivarius, Streptococcus diacetylactis, Streptococcus intermedius, Lactobacillus paracasei, Streptococcus thermophiles, Streptococcus salivarius subsp. thermophilus, Bacillus cereus, Propionibacterium freundenreichii, Oxalobacter formagenes, and Saccharomyces cerevisiae.

Bacteria used in a topical application composition may also promote health in a human or animal and may be referred to as “health-promoting bacteria.” According to one embodiment of the present teachings, a health-promoting bacteria is at least one member chosen from a group comprising Lactobacillus acidophilus, Lactobacillus reuteri, Lactobacillus plantarum, Lactobacillus casei, Lactobacillus johnsonii, Lactobacillus rahamnosus, Lactobacillus gasseri, Bifidobacterium lactis, Bifidobacterium infantis, Bifidobacterium longum, Saccharomyces boulardii, Lactobacillus salivarus, Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescsents, Bifidobacterium animalis, Bifidobacterium thermophilium, Enterococcus faecalis, Streptococcus cremoris, Streptococcus salivarius, Streptococcus diacetylactis, Streptococcus intermedius, Lactobacillus paracasei, Streptococcus thermophiles, Streptococcus salivarius subsp. Thermophilus, Bacillus cereus, Proprionibacteria freundenreichii, Bacillus coagulans (L. sporegenes), and Oxalobacter formagenes. According to the present teachings, the presence of a health-promoting bacteria in a topical application composition provides a means of delivering one or more health benefits to a human or animal that consumes a food that is coated with a topical application composition (that includes the health-promoting bacteria).

A topical application composition may include any combination of a food-preserving bacteria, a food-safety bacteria, and/or a health-promoting bacteria, to produce the benefits associated with each type of bacteria. By way of example, a topical application may include a bacteria that is a food-preserving bacteria and a food-safety bacteria as a component of a food product that is shelf-stable and safe. The topical application composition may then also include a health-promoting bacteria that delivers health benefits to a human or animal that consumes the topical application. According to one embodiment of the present teachings, a topical application includes a Pediococci (i.e., a food-safety and a food-preserving bacteria) and a Lactobacillus (i.e., a health-promoting bacteria that promotes intestinal health). According to another embodiment of the present teachings, a topical includes a Pediococci (i.e., a food-safety bacteria) and a Bifidobacteria (i.e., a health-promoting bacteria that promotes intestinal health). According to yet another embodiment of the present teachings, a topical application includes a Pediococci (i.e., a food-safety bacteria) and a Enterococcus (i.e., a health-promoting bacteria that promotes intestinal health).

As mentioned above, the bacteria in a topical application composition are in a substantially non-fermenting state. According to one embodiment of the present teachings, at least about 1×10³ cfu/g of bacteria in a topical application composition are not in a fermenting state. According to another embodiment of the present teachings, at least about 1×10⁵ cfu/g of bacteria in a topical application composition are not in a fermenting state. In certain embodiments of the present teachings, a topical application also includes at least some bacteria that are fermenting. According to one embodiment of the present teachings, up to about 1×10¹⁰ cfu/g of bacteria in a topical application composition are fermenting.

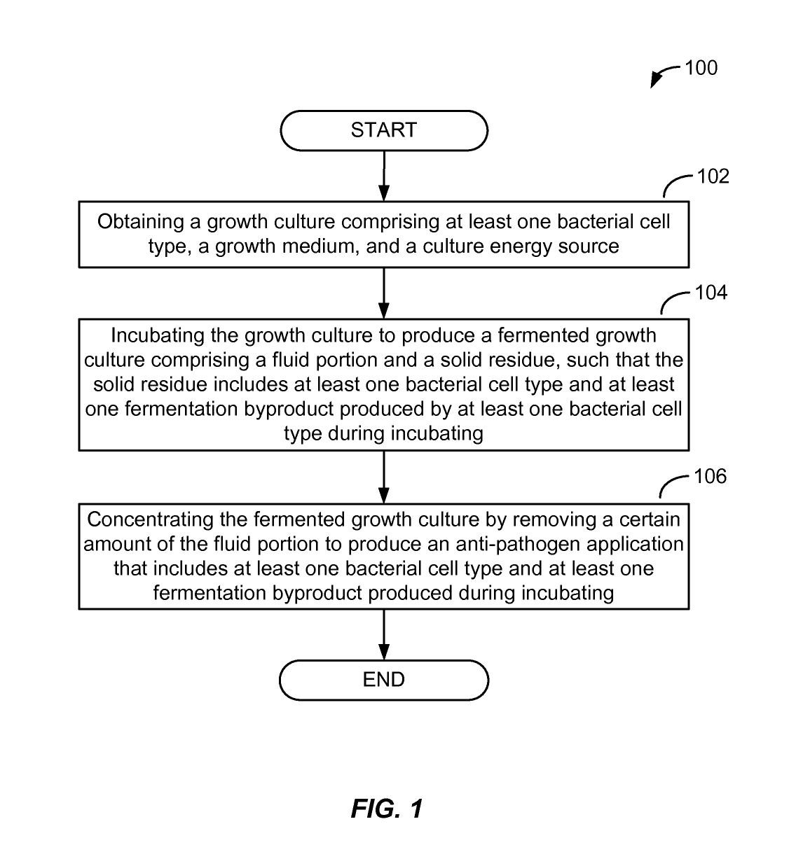

A topical application also includes one or more fermentation byproducts. Preferably, a fermentation byproduct is byproduct of fermentation that is produced during growth, metabolism, and/or fermentation of the bacteria prior to being used in a topical application composition (e.g., as described below with reference to treating step 104 of FIG. 1). The fermentation byproducts of the present teachings are thought to promote food safety and preservation by facilitating growth inhibition and/or death of one or more pathogens or food-spoilage microorganisms.

A fermentation byproduct may include at least one member chosen from a group comprising bacteriocin, pediocin, hydrogen peroxide, lactate, glycoprotein, and acid mucin. According to one embodiment of the present teachings, the fermentation byproduct includes at least one antimicrobial lactic acid producing bacterial metabolite chosen from a group comprising phenyllactic acid, 3-hydroxyphenyllactic acid, 4-hydroxyphenylactic acid, 3-hydroxy propanaldehyde, 1,2 propandiol, 1,3 propandiol, hydrogen peroxide, ethanol, acetic acid, carbon dioxide, carbonic acid, propanoic acid, butyric acid, cyclic dipeptides, cyclo(L-Phe-L-Pro), cyclo(L P-Traps-4-OH-L-Pro), 3-(R)-hydroxydecanoic acid, 3-hydroxy-5-cic dodecanoic acid, 3-(R)-hydroxy dodecanoic acid, and 3-(R)-hyroxytetradecanoic acid. According to another embodiment of the present teachings, the fermentation byproduct include at least one bacteriocin that is a lantibiotic (Class II) or a non-lantibiotic (Class II). According to yet another embodiment of the present teachings, the fermentation byproduct include at least one bacteriocin selected from a group comprising nisin A, nisin Z, nisin Q, nisin F, nisin U, nisin U2, salivarcin X, lacticin J46, lacticin 481, lacticin 3147, salivarcin A, salivarcin A2, salivarcin A3, salivarcin A4, salivarcin A5, salivarcin B, streptin, salivaricin A1, streptin, streptococcin A-FF22, BHT-Aa, BHT Ab, mutacin BNY266, mutacin 1140, mutacin K8, mutacin II, smbAB, bovicin HJ50, bovicin HC5, macedocin, plantaricin W, lactocin 5, cyctolysin, enterocin A, divercin V41, divercin M35, bavaricin, coagulin, pediocin PA-1, mundticin, piscicocin CS526, piscicocin 126/V1a, sakacin P, leucocin C, sakacin 5X, enterocin CRL35/mundticin, avicin A, mundticin I, enterocin HF, bavaricin A, ubericin A, leucocin A, mesentericin Y105, sakacin G, plantaricin 423, plantaricin C19, curvacin A/sakacin A, carnobacteriocin BM1, enterocin P, piscicoin V1b, penocin A, bacteriocin 31, bacteriocin RC714, hiracin JM79, bacteriocin T8, enterocin SE-K4, carnobacteriocin B2, SRCAM 1580, and CONCENSUS.

The fluid portion of a topical application may include one or more fluids. By way of example, growth media used during a process of producing a topical application (e.g., as explained below with reference to FIG. 1) may be a fluid in a topical application composition. Likewise, a fluid such as water or a buffer solution (e.g., saline, Butterfield's phosphate buffered solution, tris buffers, and the like), may be added to a topical application composition.

The topical application composition may be produced as a concentrate (i.e., a concentrated dose or amount of the solid residue in the topical application). According to one embodiment of the present teachings, a topical application composition has an original amount of solid residue relative to an amount of fluid portion, by weight. According to one embodiment of the present teachings, a topical application concentrate has an amount of solid residue relative to an amount of the fluid portion, by weight, that is between about 2 times and about 20 times greater than the original amount. Preferably, a topical application concentrate has a concentration of solid reside relative to the fluid portion that is about 8 times the original amount.

A concentration of bacteria in a topical application corresponds to the solid/culture ratio and may be expressed as colony forming units (“cfu”) of bacteria per unit volume or weight of a topical application, i.e., “cfu/ml” or “cfu/g,” respectively. A solid/culture ratio value of 1:1 corresponds to about 1.2×10⁹ cfu/ml, 2:1 corresponds to about 2.4×10⁹ cfu/ml, 4:1 corresponds to about 4.8×10⁹ cfu/ml or cfu/g, and 8:1 corresponds to about 9.6×10⁹ cfu/ml.

A topical application composition may be a pure-packed bacterial culture, or a “concentrate,” that includes the solid residue substantially separated from a fluid portion. According to one embodiment of the present teachings, a pure packed cell culture includes an amount of bacteria in a topical application that is between about 1×10¹¹ cfu/(gram of the topical application composition) and about 1×10¹² cfu/(gram of the topical application composition). A topical application composition that is a concentrate may also include a certain amount of fluid that is either added to the composition or is a residual amount of fluid that remains after the solid residue is separated (e.g., as explained below with reference to step 106 of FIG. 1, below).

The concentration of live bacteria in a topical application is any concentration of live bacteria sufficient to promote human or animal health, food safety, and/or food preservation. In certain embodiments of the present teachings, the number of live microorganisms applied per unit of topical application composition is at least one concentration of bacteria chosen from a group comprising between about 1,000 cfu/(gram of the topical application composition) and about 100,000 cfu/(gram of the topical application composition), between about 10,000 cfu/(gram of the topical application composition) and about 1,000,000 cfu/(gram of the topical application composition), between about 100,000 cfu/(gram of the topical application composition) and about 10,000,000 cfu/(gram of the topical application composition), between about 1,000,000 cfu/(gram of the topical application composition) and about 100,000,000 cfu/(gram of the topical application composition), and between about 100,000,000 cfu/(gram of the topical application composition) and about 10,000,000,000 cfu/(gram of the topical application composition).

Such concentrations of live bacteria in a topical application may be used to promote pathogen control and safety on a food or on an inedible surface (explained in further detail below). In other words, these concentrations of live bacteria in a topical application, when applied to a food or an inedible surface under the appropriate conditions, produce a food or an inedible surface that is substantially free of pathogens and/or food-spoilage microorganisms. According to one embodiment of the present teachings, substantially free of pathogens and/or food-spoilage microorganisms means the food has less than about 10 cfu of pathogens or food-spoilage microorganisms per gram of food. According to another embodiment of the present teachings, substantially free of pathogens and/or food-spoilage microorganisms means the inedible surface has less than 10 cfu of pathogens of food-spoilage microorganisms per cm² of inedible surface.

The pH of a topical application composition may vary. According to certain embodiments of the present teachings, a topical application has a pH value that is between about 3.0 and about 3.4, between about 3.4 and about 3.8, between about 3.8 and about 4.3, between about 4.3 and about 4.7, or between about 4.7 and about 5.1. In other embodiments of the present teachings, the topical application composition has a pH value that is sufficient to facilitate food safety and food preservation. The present teachings recognize that in food products stored with a topical application, pH is stable over time because the bacteria therein are in a substantially non-fermenting state (i.e., because lactic acid production associated with fermentation would lower the pH). Likewise, no proteolysis occurs (e.g., because bacilli growth would result in proteolysis that would increase the pH).

A topical application composition may include additional components to facilitate its use. By way of example, a topical application composition may include at least one member chosen from a group comprising flavor enhancer, palatant, food coating stabilizer, fragrance, binder, color, buffer, and coloring agent. Such components may, for example, be used to produce a topical application that is more flavorful (e.g., to facilitate use in a food) and/or more fragrant (e.g., to facilitate use on an inedible surface). Likewise, such components (e.g., a palatant) may be used to produce a food product having an improved or acceptable taste.

Further, to facilitate storage and stability of a topical application composition, and/or to slow the death of bacteria in the topical application composition, a preservative or stabilizing agent may be added. According to one embodiment of the present teachings a topical application composition is stored with at least one preservative or stabilizing agent chosen from a group comprising glycerol, dextrose, vitamin E, milk solids, sugar concentrates, propylene glycol, dimethyl sulfoxide (DMSO), mannitol, sorbitol, casein, meat concentrates, humectants, non-ionizing compounds that include many humectants, glycine betaine, sugars, sucrose, fructose, galactose, lactose, ethylene glycol, erythritol, threitol, dimethylformamide, 2-methyl-2,4-pentanediol, trehalose, Tween 80, and capsular material. According to one embodiment of the present teachings, a preservative or stabilizing agent may be added to a topical application composition to produce a preserved topical application composition having between about 30% by weight of preservative and about 70%, by weight of preservative.

The systems, methods, and compositions of the present teachings also disclose processes for preparing a topical application composition. To this end, FIG. 1 is flowchart showing certain salient steps of a process 100, according to one embodiment of the present teachings, for producing a topical application. FIG. 1 begins with a step 102, which includes obtaining a growth culture that includes at least one bacteria, a growth medium, and an energy source. A bacteria, including at least one of a food-safety bacteria, a food-preserving bacteria, or a health-promoting bacteria, is substantially similar to its counterparts described above with reference to a topical application composition.

A growth medium used to produce a topical application comprises a water-based liquid and various micro-nutrients (e.g., vitamins and minerals) that facilitate the growth of bacteria that is used in a topical application. In one embodiment of the present teachings, a growth media includes at least one member chosen from a group comprising beef broth, chicken broth, turkey broth, vegetable broth, fish broth, salmon broth, meat broth, Swanson® beef broth, Swanson® chicken broth, Swanson® seafood broth, De Man, Rogosa, and Sharpe (MRS) agar/broth, Lactobacillus Selective (LBS) agarbroth, Trypticase Soy Broth (TSB) supplemented with yeast extract and Tween 80, corn steep solids, brewers yeast, and bakers yeast.

An energy source used to produce a topical application may be at least one member chosen from a group comprising apple juice, apple juice concentrate, dextrose, dextrose monohydrate, dextrose hydride, grape sugar, D-glucose, corn sugar, sucrose, lactose, maltose, corn syrup solids, high fructose corn syrup, levulose, glucose, galactose, xylose, ribose, mannose, sorbose, amino acids, high fructose corn syrup, apple pulp, honey, sugar, maple syrup, pear juice, grape juice, orange juice, pear juice concentrate, grape juice concentrate, orange concentrate, and fruit juice. In one preferred embodiment of the present teachings, an energy source comprises dextrose or apple juice concentrate. In other embodiments of the present teachings, an energy source is any culture energy source sufficient to provide energy to grow bacteria.

Next, a step 104 includes incubating the growth culture to produce a fermented growth culture, which includes a fluid portion and a solid residue. The solid residue may include bacteria and one or more fermentation byproducts that are produced by the bacteria during this incubating step.

The temperature at which incubating step 104 is carried out may vary. In certain embodiments of the present teachings, the incubation temperature has a value that is between about 32° C. and about 35° C., between about 35° C. and about 38° C., between about 38° C. and about 41° C., between about 41° C. and about 43° C., between about 43° C. and about 46° C., or between about 46° C. and about 49° C. In other embodiments of the present teachings, incubating is carried out at more than one temperature. Incubation is preferably carried out at between about 28° C. and about 32° C., and more preferably, carried out at about 30° C.

The length of incubating step 104 may also vary. In certain embodiment of the present teachings, the length of incubation is dependent on the temperature of incubation. According to the present teachings, the length of incubation has a time value that is between about 4 hours and about 8 hours, between about 8 hours and about 12 hours, between about 12 hours and about 18 hours, between about 18 hours and about 24 hours, between about 24 hours and about 48 hours, or between about 48 hours and about 72 hours.

According to one embodiment of the present teachings, the fermented growth culture produced according to the embodiment of FIG. 1 is a topical application. According to another embodiment of the present teachings, however, the fermented growth culture is concentrated to produce a topical application, or a concentrate. To this end, a step 106 of process 100 includes concentrating the fermented growth culture by removing a certain amount of the fluid portion from the fermented growth culture to produce a topical application. As with the fermented growth culture, the topical application produced according to step 106 includes at least one bacteria and one or more fermentation byproducts in a solid residue. The relatively concentrated doses of a topical application that may be produced according to step 106, however, represent a preferred embodiment of the present teachings.

Concentrating in step 106 may be carried out by any technique or combinations of techniques well known to those of skill in the art. According to one embodiment of the present teachings, concentrating may be carried out by at least one technique chosen from a group sedimenting, centrifuging, vacuuming, decanting, drying, freeze drying, spray drying, and rotary evaporating.

According to another embodiment of the present teachings, concentrating in step 106 includes preparing multiple batches of a fermented growth culture (e.g., as described above with reference to step 104) and combining multiple solid residues separated from each fermented growth culture batch to prepare a single topical application concentrate.

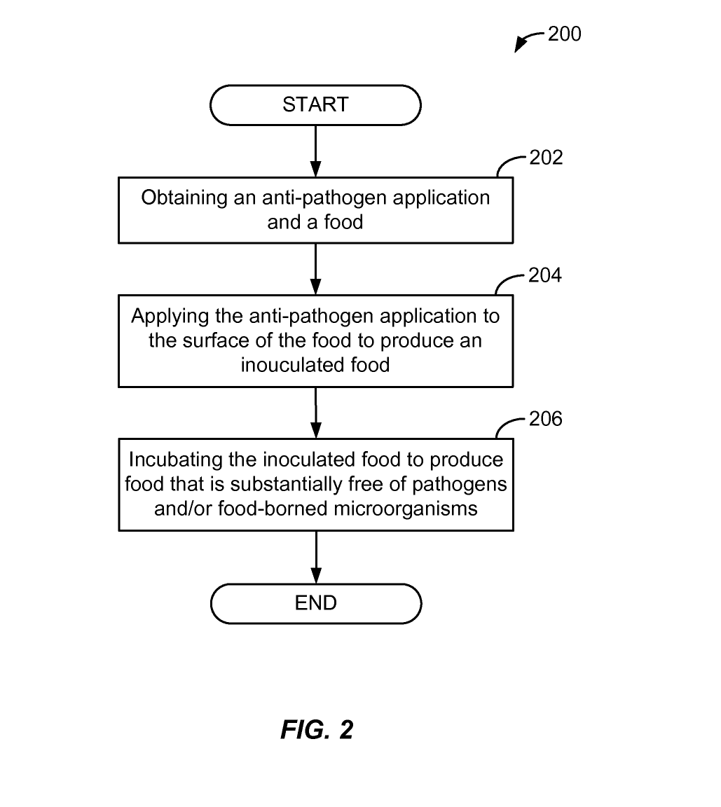

After a topical application is prepared according to the present teachings, it may be applied to a surface of a food to promote food safety and/or food preservation, including by killing and/or inhibiting the growth of one or more pathogens and/or food-spoilage microorganisms associated with food contamination. To this end, FIG. 2 is a flowchart showing certain salient steps for a process 200 for producing a food product that is substantially free of pathogens and/or food-spoilage microorganisms, according to one embodiment of the present teachings.

Process 200 begins with a step 202, which includes obtaining a topical application and a food. The topical application may include one or more food-safety, food-preserving, and/or health-promoting bacteria, which are substantially similar to their counterparts described above with reference to a topical application composition.

The embodiment of FIG. 2 contemplates obtaining any food. The food may have a moisture content that is at least about 15%, and preferably, at least about 10%. According to one embodiment of the present teachings, a food includes at least one member chosen from a group comprising kibbled food, kibbles, expanded food, pelleted food, extruded food, refrigerated food, refrigerated treats, frozen foods, frozen treats, biscuits, raw foods, fried foods and treats, soft-moist foods, soft-moist treats, pellets, fines, broken pieces, jerky-style treats, injection-molded treats, treats, supplements, prepared salad ingredients, ground fruits or vegetables, ground fruits or vegetables, prepared meals, meat, slaughtered carcasses, prepared foods, meats, meat pieces, meat chunks, fabricated meat chunks, fabricated protein chunks, livestock feeds, steam-flaked feeds, and aquaculture feeds.

Next, a step 204 includes applying, to a surface of the food, the topical application, to produce an inoculated food. Applying a topical application to a surface of a food may be carried out using any technique well known to those of skill in the art. By way of example, applying a topical application to a food includes at least one technique chosen from a group comprising coating, pouring, brushing, dribbling, spraying, soaking, misting, aerosolizing, affixing, and atomizing. In one embodiment of the present teachings, a topical application is applied to the surface of food that is tumbled in a batch-coating operation, e.g., using a paddle mixer, vibrating conveyor, screw conveyor, bucket conveyor, or some other device that results in a fluidized bed of kibbles. In another embodiment of the present teachings, a topical application is applied to a food surface using spray-on misting, e.g., as food pieces pass through a ribbon mixer. In another embodiment of the present teachings, a topical application is applied to a food surface by spray-on misting, e.g., using a screw conveyor.

In certain embodiments of the present teachings, techniques used to apply a topical application to a food surface vary based on the form of the food product. By way of example, a kibbled pet food may include a porous body having a core that is surrounded by a surface. The surface and pores on the surface are accessible to contamination with pathogens and are similarly accessible to a topical application. In such embodiments, the topical application is coated onto the surface of kibbles after extrusion and drying and before packaging. A topical application may be applied to any surface of a kibble, including any pore that enables the migration of a topical application into the interior of a kibble. In certain embodiments of the present teachings, a food surface is coated with fat, palatants, or other coating materials prior to or after application of a topical application to a surface of a kibble to facilitate surface application of the topical application.

The percentage, by weight, of a topical application applied to a surface of a food (i.e., the weight of a topical application/weight of a food piece) is any amount sufficient to promote food safety and/or food preservation. In certain embodiments of the present teachings, percentage of topical application is at least one value chosen from a group comprising between about 0.2% by weight of a food piece and about 0.5% by weight of a food piece, between about 0.5% by weight of a food piece and about 1% by weight of a food piece, between about 1% by weight of a food piece and about 1.5% by weight of a food piece, between about 1.5% by weight of a food piece and about 2% by weight of a food piece, between about 2% by weight of a food piece and about 2.5% by weight of a food piece, between about 2.5% by weight of a food piece and about 3% by weight of a food piece, between about 3% by weight of a food piece and about 5% by weight of a food piece, and between about 5% by weight of a food piece and about 10% by weight of a food piece. In other embodiments of the present teachings, the percentage, by weight, of a topical application that is applied to a surface of a food is at least one value chosen from a group comprising between about 0.01% by weight of a food piece and about 0.2% by weight of a food piece, between about 0.2% by weight of a food piece and about 1% by weight of a food piece, between about 1% by weight of a food piece and about 1.5% by weight of a food piece, between about 1.5% by weight of a food piece and about 2% by weight of a food piece, between about 2% by weight of a food piece and about 2.5% by weight of a food piece, or between about 2.5% by weight of a food piece and about 3% by weight of a food piece.

The density of food pieces on which a topical application is applied may vary, e.g., based on how a food is prepared, how it is shaped, its ingredient composition, and how a food is dried. According to certain embodiments of the present teachings, a density of food on which a topical application is applied has a value that is between about 1 g/(cm² of a food piece) and about 2 g/(cm² of a food piece), between about 2 g/(cm² of a food piece) and about 3 g/(cm² of a food piece), between about 3 g/(cm² of a food piece) and about 4 g/(cm² of a food piece), between about 4 g/(cm² of a food piece) and about 6 g/(cm² of a food piece), between about 6 g/(cm² of a food piece) and about 8 g/(cm² of a food piece), between about 8 g/(cm² of a food piece) and about 10 g/(cm² of a food piece), between about 10 g/(cm² of a food piece) and about 12 g/(cm² of a food piece), between about 12 g/(cm² of a food piece) and about 20 g/(cm² of a food piece), between about 20 g/(cm² of a food piece) and about 40 g/(cm² of a food piece), between about 40 g/(cm² of a food piece) and about 60 g/(cm² of a food piece), between about 60 g/(cm² of a food piece) and about 80 g/(cm² of a food piece), or between about 80 g/(cm² of a food piece) and about 100 g/(cm² of a food piece).

Further, the present teachings recognize that the shape of food pieces (e.g., kibbled pet food) on which a topical application is applied vary widely. The present teachings recognize that a topical application may be applied on food pieces of any shape, so long as the topical application may be applied to the surfaces of those food pieces.

According to one embodiment of the present teachings, a food surface on which a topical application is applied has a temperature value that is between about 0° C. and about 20° C., between about 20° C. and about ° C., between about 30° C. and about 35° C., between about 35° C. and about 48° C., between about 40° C. and above 45° C., between about 45° C. and about 50° C., between about 50° C. and about 55° C., between about 55° C. and about 50° C., or between about 60° C. and about 65° C. In alternate embodiments of the present teachings, a food surface on which a topical application is applied has a temperature value that is between about 35° C. and about 48° C., or between about 40° C. and about 45° C.

Likewise, applying a topical application to a food may be facilitated by including, in the topical application, one or more surfactants, binders, or ingredients that improve the ability of the topical application to coat onto a surface of a food piece. To this end, a topical application may further include at least one member chosen from a group comprising lecithin, glycerol, propylene glycol, betonies, and trimethylglycine.

Next, a step 206 includes incubating the inoculated food to produce a food that is substantially free of pathogens and/or food-spoilage microorganisms. In one embodiment of the present teachings, incubating the inoculated food, i.e., having a topical application applied thereto is carried out at a temperature that is between about 42° C. and about 50° C. for a time that is between about 16 hours and about 20 hours. In another embodiment of the present teachings, incubating is carried out at a temperature that is between about 57° C. and about 60° C. for a time that is between about 4 hours and about 8 hours. In yet another embodiment of the present teachings, incubating is carried out at a temperature that is between about 29° C. and about 32° C. for a time that is between about 48 hours and about 72 hours. The present teachings recognize that such incubation conditions promote elimination of pathogens and/or spoilage microorganisms in food on which a topical application is applied. In certain embodiments of the present teachings, however, an incubating step is not required.

According to the embodiment of FIG. 2, a food product that is substantially free of pathogen and/or spoilage microorganisms, i.e., a food product that is safe and/or shelf-stable, is produced. According to one embodiment of the teachings, a food product produced according to the embodiment of FIG. 2 has less than about 100 cfu/mg of spoilage microorganisms and/or pathogens on or in the food product. A pathogen may be at least one member chosen from a group comprising Salmonella, pathogenic Escherichia coli, Shigella, Listeria monocytogenes, Staphylococcus aureus, Campylobacter jejuni, Campylobacter coli, Clostridium botulinum, Clostridium perfringens, Trichinella spiralis, Vibrio parahaemolyticus, Vibrio cholera. A food-spoilage microorganism may be at least one member chosen from a group comprising Rhizopus nigricans, Penicillium, Aspergillus niger, Bacillus subtilis, Enterobacter aerogenes, Saccharomyces, Zygosaccharomyces, Micrococcus roseus, Aspergillus, Rhizopus, Erwinia, Botrytis, Rhodotorula, Alcaligenes, Clostridium, Proteus vulgaris, Pseudomonas fluorescens, Micrococcus, Lactobacillus, Leuconostoc, Alcaligenes, Flavobacterium, Proteus, and Acetobacter.

It is noteworthy that present food manufacturing environments strive to reduce the level of live bacteria that is present in, throughout, and on the surface of food. Current manufacturing environments are typically set up with routine procedures to monitor the plant's environment to assure the products produced are free of pathogens and/or spoilage microorganisms. Manufacturing personnel are often instructed to clean and disinfect manufacturing personnel routinely while further striving to prevent infestation of products through their own interaction with the food and equipment. The unplanned but nonetheless resulting outcome is the mindset of killing all bacteria within the manufacturing environment and on the food in order to avoid the risk of pathogen and/or spoilage microorganism infestation. In fact, killing all bacteria unintentionally creates an environment that is easier for pathogens and food-spoilage microorganisms to grow due to reduced competition from other bacteria.

The systems, methods, and compositions of the present teachings are, therefore, counterintuitive to the mindset of preventing bacteria contamination associated with conventional techniques. Rather than indiscriminately, and with significant effort and expense, undertaking the effort to kill all bacteria in a food-manufacturing environment, often by repeatedly sterilizing food and/or equipment, the present teachings promote using a topical application to selectively deactivate pathogenic and food-spoilage microorganisms using live bacteria, preferably in its substantially non-fermenting bacteria.

Further, the present teachings provide the advantage of combining a food with a topical application that may be packaged to produce shelf-stable food product that is substantially free of pathogens and/or food-spoilage microorganisms. The present teachings recognize that topical applications, and in particular, topical application concentrates, are more lethal to pathogens and/or food-spoilage microorganisms, than simple fermentation solutions. In other words, they provide “enhanced killing power” of pathogens and food-spoilage microorganisms.

Using a topical application to sterilize food provides several key advantages, including: (1) substantially killing or inhibiting the growth of pathogens and/or food-spoilage microorganisms; (2) contributing minimal to no off-taste when applied to the surface of food; (3) minimizing the chance of food being re-infested with pathogens and/or food-spoilage microorganisms; (4) inexpensive/low capital solutions for use in food manufacturing environments; and (5) reduction or elimination of the need to use harmful or expensive chemicals on a food product. Indeed, a topical application has no known detrimental impact associated with its use on a finished product or ingredient. Further, use of a topical application avoids problems associated with using fermentation to sterilize food, as fermentation often results in a buildup of excessive amounts of acid during fermentation, thus producing an undesirable taste in the food that is not found in food treated with a topical application that provides bacteria in a substantially non-fermenting state.

Use of a topical application also provides key advantages that may be useful to existing food-processing techniques. By way of example, during a batch application of a topical application to the surface of food that is being processed, food pieces may travel through various machinery and experience vibration or abrasive forces as they are being coated and packaged. Typically, at least some of the food pieces fall off during such processing (e.g., in association with fines or broken kibble pieces). The broken food pieces are often susceptible to pathogen growth in growth niches that end up contaminating the food pieces, particularly where water activity is in excess of about 0.85 (which generally supports Salmonella growth). Such issues arise under many circumstances, including but not limited to when water-based palatants are applied to dried kibbles, where there is a significant change in temperature to the product during production, when food pieces (e.g., kibble) are packed into a hermetically sealed package, or when food pieces are more than about 5° C. greater than ambient temperatures during packing. However, where the broken food pieces had been coated with a topical application, according to the present teachings, pathogen activity and/or the activity of food-spoilage microorganisms on those food pieces is reduced. This is particularly advantageous because often, small amounts (typically less than about 10%) of the fines and broken pieces of food resulting from food production are normally re-worked into future batches of food in order to prevent waste. Thus, an unintended benefit of a topical application that has been applied to food that produces fines or broken pieces is that the topical application may limit the growth of pathogens such as salmonella in the pile of fines or broken pieces, even where water is available to support growth of pathogens and/or food-spoilage microorganisms. The topical application associated with the fines and broken pieces that are re-worked into future batches of products could also have the unintended benefit of providing anti-salmonella components, such as lactic acid and bacteriocins capable of decreasing pathogens such as Salmonella, associated with the interior of the food pieces.

Further, use of a topical application is noteworthy given the considerable interest that now exists in using certain bacteria (i.e., probiotics) for improving the health of a human or animal. Accordingly, a further benefit of applying a topical application on a food is to provide bacteria on a food to be consumed by a human or animal, thus delivering health benefits associated with beneficial bacteria to that host. In some situations intestinal health may be improved. In other situations, dental health, joint and mobility health, reduced inflammation and improved mood benefits may arise from the ingestion of certain bacteria that have been used to confer a benefit to the surface of the food.

In another aspect, the systems, methods, and compositions disclosed herein are also used to promote sterility or sanitation (i.e., substantially killing and/or inhibiting growth of pathogens and/or food-spoilage microorganisms) of inedible surfaces. By way of example, a topical application may be applied to an inedible surface for the purpose of reducing contamination (e.g., by pathogens and/or food-spoilage microorganisms) risk during the preparation of food, feed, beverages, beauty care products, health care products, drugs, air handling systems, and other application. This action limits the risk of infection or contamination by pathogens and/or food-spoilage microorganisms, and also avoids any undesirable bacterial metabolites/toxins produced from their growth.

The present teachings contemplate use of a topical application to sterilize or sanitize any inedible surface, including a surface of a human or animal. According to one embodiment of the present teachings, an inedible surface is at least one member chosen from a group comprising pipe, tool, chopper, grinder, hammer mill, roller mill, flaker, emulsifier, blender, block pre-breaker, block breaker, extruder, coating equipment, APEC coater, spray bar, dryer, conveyor, pellet mill, steam flaker, vortex mill, storage bin, band saw, knife, cutting surface, countertop, wood chopping block used in food preparation, stainless steel counter top, counter top, bathroom, wet bar, alcohol serving establishment, drainage system, disposal system, sink drain, kitchen sink, toilet, toilet bowl rim, bath drain, bath tub, garbage can, barn environment, barn stall, horse stall, livestock exhibition hall, livestock bedding area, retention pond, sewage holding tank, areas around sewage holding tanks, dog kennel, dog cage, cat cage, cat carrier, dog carrier, cattery, automotive garage, air recirculation system on jet airliner, shrimp shell after meat has been removed, fish parts after fillets have been removed, animal parts after meat has been removed, human hair, dog hair coat, diaper, cream, skin, dermatitis, psoriasis, eczema, bed sore, dentifrice, oral rinse, vaginal rinse, douche, tampon, feminine pad, waste pail, garbage can, dumpster, waste handling container, commercial waste management vehicle, garbage truck, waste hauling equipment, waste capture equipment, bin, can, vehicle, tote, conveyer, waste processing equipment, waste, under-arm, vagina, foot, outer ear, diaper, stainless steel equipment such as pipes, tools, choppers, grinders, hammer mills, roller mills, flakers, emulsifiers, blenders, block pre-breakers, block breakers, transfer belts, pneumatic transfer equipment, bucket elevators, screw elevators, intermediate storage bins, finished product storage bins, weigh scales, dry filling equipment, extruders, coating equipment, APEC coaters, spray bars, dryers, conveyors, pellet mills, steam flakers, vortex mills, storage bins, band saws, knives, cutting surfaces, counters, and countertops.

In certain embodiments of the present teachings, the number of bacteria contained within a topical application that is applied to an inedible surface associated with food production is a value that is between about 1×10⁵ cfu/(cm² of inedible surface) and about 5×10⁵ cfu/(cm² of inedible surface), between about 5×10⁵ cfu/(cm² of inedible surface) and about 1×10⁶ cfu/(cm² of inedible surface), between about 1×10⁶ cfu/(cm² of inedible surface) and about 1.5×10⁶ cfu/(cm² of inedible surface), between about 1.5×10⁶ cfu/(cm² of inedible surface) and about 2×10⁶ cfu/(cm² of inedible surface), between about 2×10⁶ cfu/(cm² of inedible surface) and about 2.5×10⁶ cfu/(cm² of inedible surface), between about 2.5×10⁶ cfu/(cm² of inedible surface) and about 3×10⁶ cfu/(cm² of inedible surface), between about 3×10⁶ cfu/(cm² of inedible surface) to about 4×10⁶ cfu/(cm² of inedible surface) and between about 4×10⁶ cfu/(cm² of inedible surface) and about 1×10⁹ cfu/(cm² of inedible surface). In alternate embodiments of the present teachings, the number of bacterial cells in a topical application applied onto various areas used in a food production facility is a value that is between about 5×10⁵ cfu/(cm² of inedible surface) and about 1×10⁶ cfu/(cm² of inedible surface), between about 1×10⁶ cfu/(cm² of inedible surface) and about 1.5×10⁶ cfu/(cm² of inedible surface), between about 1.5×10⁶ cfu/(cm² of inedible surface) and about 2×10⁶ cfu/(cm² of inedible surface), and between about 4×10⁶ cfu/(cm² of inedible surface) and about 1×10⁹ cfu/(cm² of inedible surface).

The amount of topical application that is applied to an inedible surface may vary based on the degree of activity needed to inactivate pathogens and/or food-spoilage microorganisms on the inedible surface. In certain embodiments of the present teachings, a topical application is applied to an inedible surface at a value that is between about 0.25 mg/(cm² of inedible surface) and about 1 mg/(cm² of inedible surface), between about 1 mg/(cm² of inedible surface) and about 2 mg/(cm² of inedible surface), between about 2 mg/(cm² of inedible surface) and about 3 mg/(cm² of inedible surface), between about 3 mg/(cm² of inedible surface) and about 4 mg/(cm² of inedible surface), between about 4 mg/(cm² of inedible surface) and about 6 mg/(cm² of inedible surface), between about 6 mg/(cm² of inedible surface) and about 8 mg/(cm² of inedible surface), between about 8 mg/(cm² of inedible surface) and about 10 mg/(cm² of inedible surface), between about 10 mg/(cm² of inedible surface) and about 12 mg/(cm² of inedible surface), or about 12 mg/(cm² of inedible surface) and about 20 mg/(cm² of inedible surface).

As but one example, the topical application may be applied to shrimp shells after the meat has been removed. This serves to lessen the likelihood the shells will degrade. As another example, the topical application may also be applied to the hair on a human's head for the purpose of straightening curly hair. As yet another example, the topical application may be applied to dog hair coats to reduce the smell of “wet dog.” As yet another example, the topical application may be applied to diapers or creams to reduce irritation or infections of the buttocks causing rashes and other discomfort. As yet another example, the topical application may also be applied to under-arms, vagina, feet, and outer ears for the purpose of lessening rashes or preventing undesirable bacteria growth.

According to other embodiments the present teachings, a topical application is applied to the surface of a human or animal to promote health. In particular, a topical application may be applied to a surface of a human or animal to disinfect or sterilize an injured or unhealthy human or animal surface. According to one embodiment of the present teachings, a topical application is applied directly or in the form of a cream. The topical application may used, for example, to treat or alleviate dermatitis, psoriasis, eczema, and other forms of skin maladies. A topical application may also be used in hospital environments to treat bed sores caused by staphylococcus infections. A topical application may also be applied to dentifrices, oral rinses and vaginal rinses and douches. A topical application may further be applied to tampons and feminine pads.

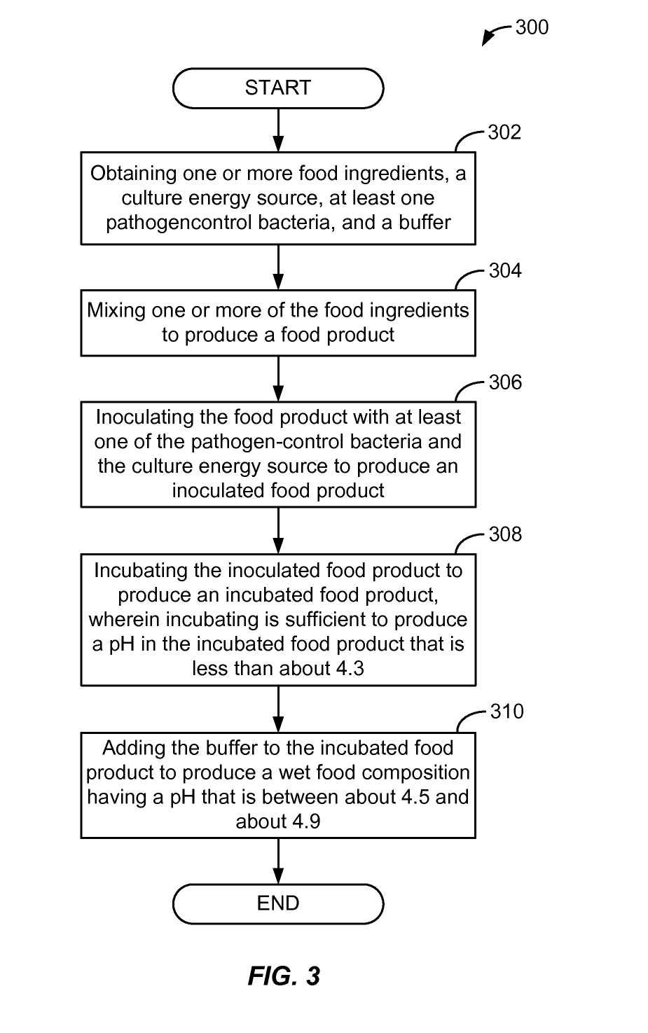

In yet another aspect, the present teachings disclose systems, methods, and compositions associated with producing a wet food composition. To this end, FIG. 3 is flowchart of a process 300 showing certain salient steps, according to one embodiment of the present teachings, for producing a wet food composition. Process 300 begins with a step 302, which includes obtaining one or more food ingredients, a culture energy source, at least one food-safety bacteria and/or at least one food-preserving bacteria, and a buffer. A culture energy source, a pathogen control bacteria, and a food-spoilage bacteria are substantially similar to their counterparts described above with reference to a topical application. A food ingredient is any edible substance that has a moisture content of at least about 15% by weight of the edible substance, and any ingredient combined with food, so long as the combined food ingredients maintains a moisture content of at least about 15%. A buffer may be any buffer sufficient to neutralize acid produced during fermentation. By way of example, a buffer is at least one member chosen from a group comprising calcium carbonate, sodium bicarbonate, carbonic acid, pyrophosphates, sodium acid pyrophosphate, malic acid, potassium citrate, apple juice concentrate, acetic acid, sodium acetate, calcium malate, sodium citrate, calcium citrate, monopotassium phosphate, potassium tartrate, vinegar, and tricalcium phosphate.

Next, a step 304 includes mixing one or more of the food ingredients to produce a food product. Mixing may be carried out by any technique well known to those of skill in the art.

Next, a step 306 includes inoculating the food product with at least one food-safety bacteria and/or at least one food-preserving bacteria to produce an inoculated food product. Inoculating may be carried out by any technique well known to those of skill in the art. According to one embodiment of the present teachings, a food product may also be inoculated with health-promoting bacteria.

Next, a step 308 includes incubating the inoculated food product to produce an incubated food product. Incubating may be carried out during conditions that are sufficient to produce a pH in the incubated food product that is less than about 4.5, and preferably less than about 4.3. Such reduction in pH may largely be driven by the production of lactic acid produced by fermenting bacteria on the inoculated food product. According to one preferred embodiment of the present teachings, the inoculated food product is incubated for a time that is between about 16 hours and about 20 hours at a temperature that is between about 42° C. and about 50° C. to cause the pH of the food to be reduced to less than pH 4.3.

This reduction in pH, while promoting food safety and food preservation, may also produce a food product that, while edible, is not well tolerated by some animals (e.g., dogs, which are prone to suffer from emesis due to the consumption of relatively low-pH foods, or humans who may consider highly acidic foods undesired or unpalatable). To address this, process 300 of FIG. 3 next includes an optional step 310, which includes adding the buffer to the incubated food product to produce a wet food composition having a pH that is between about 4.5 and about 4.9. The present teachings recognize that use of a buffer in step 310 decreases the acid load on the wet food composition, thus producing a food composition that not only is substantially free of pathogens and/or food-spoilage microorganisms, but is also generally well tolerated by animals and humans.

Further steps or components may be used to improve the quality of a wet food composition. According to one embodiment of the present teachings, a salt or a salt-like substance is added (e.g., in conjunction with step 304) for the purpose of extracting sugars, cellular nutrients, proteins, polypeptides and amino acids from a food to aid in buffering the food matrix. Salt further serves to isolate fat from meat and thus prevents fat from inhibiting fermentation of the bacteria in the wet food composition. Salt also provides the benefit of enhancing the flavor of the food. A salt or salt-like substance may include at least one member chosen from a group comprising sodium chloride, potassium chloride, sea salt, and calcium chloride.

Normal processing conditions of wet foods involve high amounts of heating that indirectly provides chemical reactions of various food components that improve the flavor profile of said food. It has been surprisingly found that filling the product at a lower temperature, about 100° F. to 135° F., results in improved product taste and acceptance.

According to another embodiment of the present teachings, a binder and/or a syneresis-controlling substance is added (e.g., in conjunction with step 304). A binder or a syneresis-controlling substance may be at least one member chosen from a group comprising pea powder, gum arabic, guar gum, hydrocolloids, carboxymethylcellulose, locust bean gum, cassia gum, carageenans, iota-carageenan, kappa-carageenan, milk, milk products, milk proteins, casein, pork plasma, textured vegetable proteins, glutens, corn gluten, wheat gluten, starches, corn starch, rice starch, potato starch, tapioca starch, sorghum starch, oat starch, soy, soy protein, soy protein concentrate, soy protein isolate, egg, egg derivatives, transglutaminase, gelatins, and polysaccharides. In particular, such substances may be useful in high-moisture foods for mitigating the effects of syneresis that occur with meat-based food products.

According to another embodiment of the present teachings, a wet food composition may be preserved to facilitate shelf-stability. By way of example, a bacteria (e.g., Pediococci) and a culture energy source (e.g., dextrose) may be used to stabilize a food such as meat, vegetables, fruit, pet food, pet treats or any mixture thereof. By way of another example, a food stabilizing bacteria (e.g., Pediococci), a health promoting bacteria (e.g., Lactobacillus, Bifidobacterium), and an energy source (e.g., dextrose, sucrose), may be used to stabilize a food such as meat, vegetables, fruit, pet food, pet treats or any mixture thereof. By way of yet another example, a food stabilizing bacteria (e.g., Pediococci), a health promoting bacteria (e.g., Lactobacillus, Bifidobacterium), a culture energy source (e.g., dextrose, sucrose), and a buffering agent (e.g., calcium carbonate, sodium bicarbonate) may be used to stabilize a food such as meat, vegetables, fruit, pet food, pet treats or any mixture thereof. By way of yet another example, wet foods (e.g., food containing moisture in amount that is greater than 15% by weight of the food) may be preserved using a food stabilizing bacteria (e.g., Pediococci) to become shelf stable by obtaining a low pH (i.e., less than 4.4) that is neutralized (i.e., at least 0.2 pH units higher) to provide a shelf-stable food that is comprised at least in part of health promoting bacteria (e.g., Lactobacillus or Bifidobacterium).

The systems, methods, and compositions related to the wet food composition of the present teachings provide several advantages over conventional techniques for producing shelf-stable wet food products, such as retort or autoclave processes. Drawbacks associated with such processes include the need for high amounts of heating, lengthy exposure of the food to the heat, and in some cases, high amounts of pressure. The significant amount of heating and time required for these techniques not only require costly manufacturing equipment, they also alter the nutrient quality, appearance, and texture of foods in an undesirable manner. As such, the wet food composition of the present teachings overcomes the drawbacks of retort and/or autoclave processes, as its use is considerably simpler and less expensive, while producing highly palatable food products and shelf-stable food products. The wet food composition of the present teachings may also be used to provide safe food that may be packed ready to eat.

Likewise, the use of a buffer in producing a shelf-stable wet food composition avoids drawbacks associated with other conventional food preservation and food safety techniques, and in particular, fermentation. To this end, the present teachings disclose a means to lessen the sour taste in a food product that is inoculated with bacteria that produces lactic acid, while still obtaining the benefit of high acid levels through the use of a buffer agent after the fermentation process is complete (but before the food product is packaged and then sealed). The present teachings also further overcome product taste issues by surprisingly identifying an optimal product filling temperature that not only improves product taste and acceptance, but also enables the fermented food to serve as a delivery vehicle for health-promoting bacteria.

Certain examples are set forth below. These examples provide values for various measured parameters and data. These values and data are reported as approximate values.

### EXAMPLES

### Example 1

### Application of a Fermented Meat Broth to Deactivate Salmonella Surrogates Inoculated on Fruits and Vegetables

Example 1 shows applying a topical application to fruits and vegetables to reduce pathogenic activity, according to one embodiment of the present teachings.

Dried apples, dried and diced carrots, and dried and sliced green beans were vacuum infused with canola oil at the level of about 3% application rate, i.e., to produce vacuum-infused dried apples, dried and diced carrots, and dried and sliced green beans, that have a concentration, by weight, of about 3% canola oil.

Separately, four E. coli strains (ATCC BAA 1428, BAA 1429, BAA 1430, and BAA 1431) were individually grown in about 200 ml Trypticase Soy Broth (TSB) with about 1% dextrose for about 48 hours at 35° C. according to a modified method (Niebuhr, S. E., A. Laury, G. R. Acuff and J. S. Dickson. 2005). Evaluation of non-pathogenic surrogate bacteria as process validation indicators for Salmonella enteric for selected antimicrobial treatments, cold storage and fermentation in meat. (FSIS. USDA).

The E. coli cells grown on the TSB 1% dextrose media were harvested via refrigerated centrifugation and then washed with sterile cold Butterfields Phosphate Buffer. All four strains of E. coli were combined together and spray inoculated, using atomizer spray bottles, onto the surface of fruits and vegetables to provide at least about 100,000 cfu/(gram of food product). The E. coli served as the Salmonella surrogate bacteria used in this experiment.

Additionally, a number of treatments were created and then evaluated to compare the influence of a fermentation culture to combat the growth of the E. coli strains on the vacuum infused fruits and vegetables. The composition and process for making these treatments are described below.

Treatment 1 of Table 1 involved the use of a fermentation based on the use of mechanically deboned salmon meal (MDM salmon). The MDM salmon was in a ground form. The MDM salmon was inoculated with Pediococcus acidilactici and P. pentosaceus at the level of about 1×10⁷ cfu/(gram of MDM salmon). The inoculated MDM salmon was placed in a sealed container and then placed in an environment at about 40° C. for about 48 hours to allow it to ferment. The fermented MDM salmon was applied to the dried fruits and vegetables and evaluated as the first treatment noted in Table 1.

Treatment 2 of Table 1 involved the use of a digested protein source based on the use of mechanically deboned salmon meal (MDM salmon). The MDM salmon was in a ground form and was diluted with water (about 50% by weight of MDM salmon and about 50% by weight of water). Hydrochloric acid was added to the MDM salmon at a sufficient level to achieve a pH of about 2.0. The acidified MDM salmon was placed in a sealed container and then placed in an environment at about 80° C. for about 0.5 hours to allow it to digest. The digested MDM salmon was applied to the dried fruits and vegetables and evaluated as the second treatment noted in Table 1.

Treatment 3 of Table 1 involved the use of a fermentation based on the use of beef broth. The beef broth was inoculated with Pediococcus acidilactici and P. pentosaceus at the level of 1×10⁷ cfu/(gram of beef broth). The inoculated beef broth was placed in a sealed container and then placed in an environment at about 40° C. for about 48 hours to allow it to ferment. The fermented beef broth was applied to the dried fruits and vegetables and evaluated as the third treatment noted in Table 1.

Treatment 4 of Table 1 involved the use of no fermentation or digested protein source. The lack of applying a fermentation or digested protein source allowed the evaluation of the dried fruits and vegetables in the presence of E. coli as a reference (i.e., control) treatment and is noted as the fourth treatment in Table 1.

After fermentation (or digestion) was completed, the samples of fruit and vegetables to be treated were inoculated with E. coli and then dried to room temperature for about 15 minutes. The inoculated fruit and vegetables samples were then sprayed with various test treatments (see Table 1). The treated samples were then stored at room temperature (between about 20° C. and about 22° C.). At various time intervals E. coli were enumerated on Violet Red Bile Agar at about 35° C. and 37° C. for 24 hours.

TABLE 1
Description of Test Treatments

| | | | Treatment Addition to |
|---|---|---|---|
| | Treatment | | Dried Fruits and |
| Treatment # | Name | Treatment Composition | Vegetables |
| 1 | Fermented | Mechanically deboned salmon meal | 6.7 g of the fermented |
| | salmon | was inoculated with Pediococcus | salmon meat was added to 50 g |
| | meal on | acidilactici and P. pentosaceus and | of dried fruits and |
| | Fruits & | fermented at 40° C. for 48 h. | vegetables vacuum infused |
| | Vegetables | | with 3% canola oil and 1 g of |
| | | | Butterfield's Phosphate |
| | | | Buffer containing E. coli. |
| 2 | Digested | Digested salmon was obtained by | 4.9 g of the digested salmon |
| | salmon | diluting mechanically deboned | meal was added to 50 g of |
| | meal on | salmon meal with water (1 part | dried fruits and vegetables |
| | Fruits & | salmon meal:1 part water) and | vacuum infused with 3% |
| | Vegetables | adding hydrochloric acid to adjust | canola oil and 1 g of |
| | | pH to 2 and further heating this | Butterfield's Phosphate |
| | | mixture at 80° C. for 0.5 h. | Buffer containing E. coli. |
| 3 | Fermented | Fermented beef broth was obtained | 5 g of the fermented beef |
| | beef broth | by inoculating beef broth with | broth was added to 50 g of |
| | on Fruits & | Pediococcus acidilactici and P. pentosaceus | dried fruits and vegetables |
| | Vegetables | and incubating at 40° C. | vacuum infused with 3% |
| | | for 48 h. | canola oil and 1 g of |
| | | | Butterfield's Phosphate |
| | | | Buffer containing E. coli. |
| 4 | Fruits & | No treatment composition was | No treatment composition |
| | Vegetables | created. | was added to 50 g of dried |
| | Control | | fruits and vegetables vacuum |
| | | | infused with 3% canola oil 1 g |
| | | | of Butterfield's Phosphate |
| | | | Buffer containing E. coli. |

*The values reported above are approximate values.

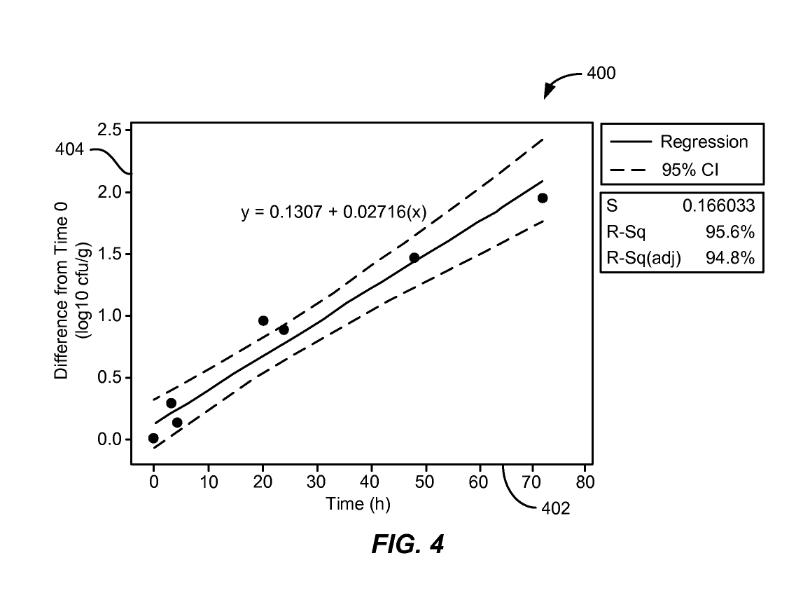

FIG. 4 is a graph 400 showing death of salmonella surrogates on fruit and vegetables coated with fermented mechanically deboned salmon meal (Treatment 1). An x-axis 402 represents the length of time (in hours) the fermentation culture was applied to the fruits and vegetables. A y-axis 404 represents the amount (log₁₀ cfu/g) of Salmonella surrogate (i.e., E. coli) death that occurred compared to the original level of E. coli applied to the fruit and vegetables. The linear regression line was highly significant (P<0.01).

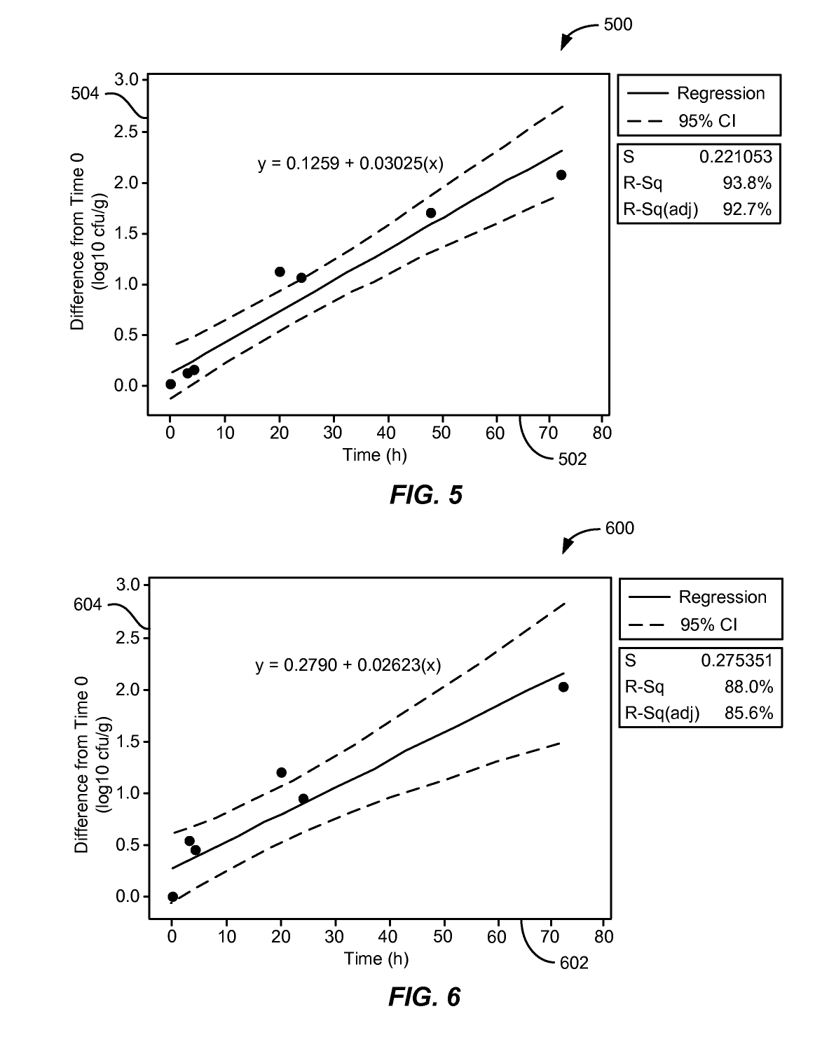

FIG. 5 is a graph 500 showing death of salmonella surrogates on fruits and vegetables coated with digested mechanically deboned salmon meal (Treatment 2). An x-axis 502 represents the length of time (in hours) the fermentation culture was applied to the fruits and vegetables. A y-axis 504 represents the amount (log₁₀ cfu/g) of Salmonella surrogate (i.e., E. coli) death that occurred compared to the original level of E. coli applied to the fruit and vegetables. The linear regression line was highly significant (P<0.01).

FIG. 6 is a graph 600 showing death of salmonella surrogates on fruits and vegetables coated with fermented beef broth (Treatment 3). An x-axis 602 represents the length of time (in hours) the fermentation culture was applied to the fruits and vegetables. A y-axis 604 represents the amount (log₁₀ cfu/g) of Salmonella surrogate (i.e., E. coli) death that occurred compared to the original level of E. coli applied to the fruit and vegetables. The linear regression line was highly significant (P<0.01).

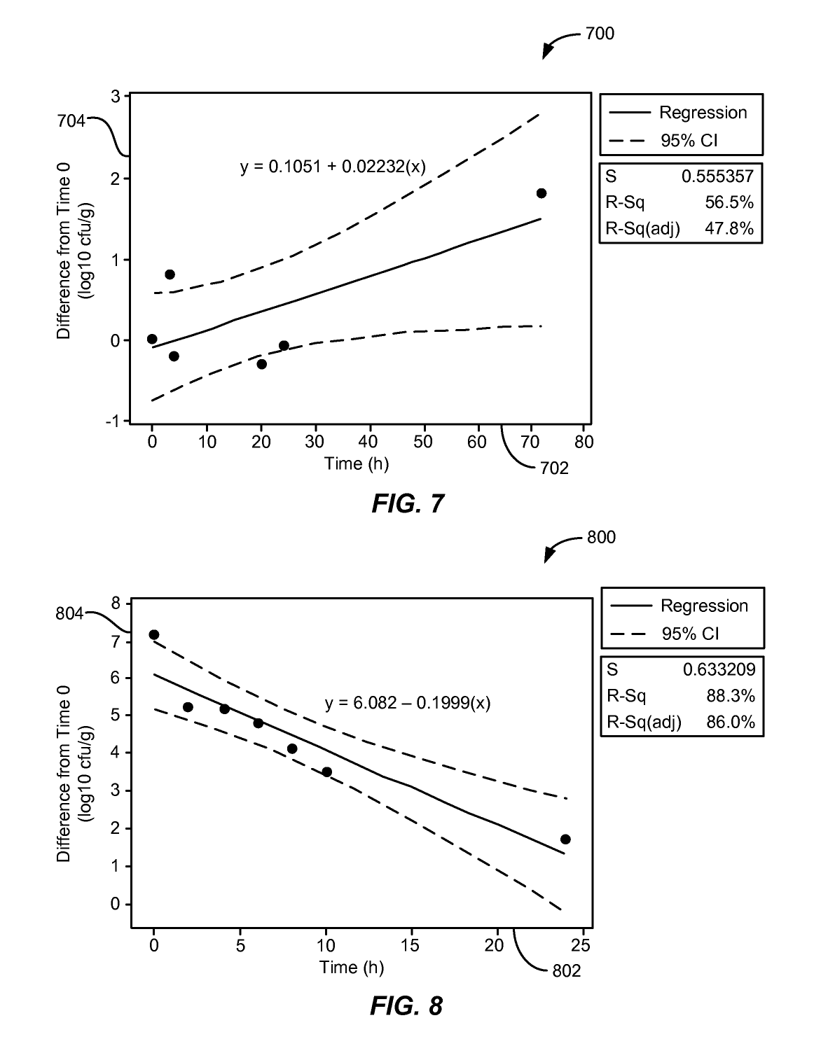

FIG. 7 shows a graph 700 showing impact of salmonella surrogates on fruits and vegetables without treatment (Treatment 4). An x-axis 702 represents the length of time (in hours) the fermentation culture was applied to the fruits and vegetables. A y-axis 704 represents the amount (log₁₀ cfu/g) of Salmonella surrogate (i.e., E. coli) death that occurred compared to the original level of E. coli applied to the fruit and vegetables. The linear regression line was moderately significant (P=0.05).

Treatments 1, 2 and 3 resulted in robust inactivation of Salmonella surrogate bacteria on fruits and vegetables as reflected by statistically significant linear regressions (p<0.01) and high adjusted r-squared values (R²>0.85; see FIGS. 4 to 6), while Treatment 4 resulted in modest death of Salmonella surrogate bacteria as reflected by a moderate significance (p=0.05) and adjusted r-squared value (R²=0.49; see FIG. 7). The reason that Treatment 4 even exhibited a modest reduction in Salmonella surrogate bacteria is likely due to an unexpectedly low pH of the source of dried fruits and vegetables used in this experiment (see Table 2).

TABLE 2
pH of Individual Fruit and Vegetable Sources

| Sample ID | Green Beans | Apple | Carrot |
|---|---|---|---|
| 1 | 4.92 | 4.38 | 5.03 |
| 2 | 4.96 | 4.30 | 5.14 |
| 3 | 4.91 | 4.29 | 5.05 |

*pH solution test involved placing 25 g of mixture into 225 ml of distilled water and then measuring pH, and all values reported in this table are approximate values.

The linear regression procedure was used to determine how long it would take to obtain 3 log₁₀ reduction in the Salmonella surrogates. Results from treatments 1, 2 and 3 represented in Table 1 were composited and used to predict the length of time in which the Salmonella surrogates would be deactivated. The results of a regression analysis of this composited data are noted in Table 3. The linear regression indicates that a 3 log₁₀ reduction in Salmonella surrogates would occur after about 108 hours of fermentation storage.

TABLE 3
Linear Regression Prediction of the Level of Salmonella Surrogate
Reduction Over Time

| Time (h) | Log₁₀ Reductions (at 20 to 22° C.) |
|---|---|
| 96 | 2.80 |
| 108 | 3.11 |
| 120 | 3.43 |
| 144 | 4.06 |

*All values reported in this table are approximate values.

### Example 2

### Application of Meat Broth to Deactivate Salmonella Surrogates Inoculated on Kibbles

Applying a topical application to pet food kibbles to reduce pathogenic activity, according to one embodiment of the present teachings, is shown by Example 2.

Treatment 1 of Table 4 was obtained by fermenting a growth media source that was based on chicken broth. The chicken broth contained about 2% by weight of dextrose and was inoculated with Pediococcus acidilactici and P. pentosaceus at the level of 1×10⁷ cfu/(gram of chicken broth). The inoculated chicken broth was placed in a sealed container and then placed in an environment at about 40° C. for about 48 hours to allow it to ferment. Upon fermentation, the chicken broth fermentation culture was inoculated with E. coli as described in Table 4.

Treatment 2 of Table 4 was obtained by fermenting a growth media source that was based on beef broth. The beef broth was inoculated with Pediococcus acidilactici and P. pentosaceus at the level of 1×10⁷ cfu/(gram of beef broth). The inoculated beef broth was placed in a sealed container and then placed in an environment at about 40° C. for about 48 hours to allow it to ferment. Upon fermentation, the beef broth fermentation culture was sprayed onto kibbles, which were then inoculated with E. coli as described in Table 4.

Treatment 3 of Table 4 was obtained by fermenting a growth media source that was based on chicken broth. The chicken broth was inoculated with Pediococcus acidilactici and P. pentosaceus at the level of about 1×10⁷ cfu/(grams of chicken broth). The inoculated chicken broth was placed in a sealed container and then placed in an environment at about 40° C. for about 48 hours to allow it to ferment. Upon fermentation, the chicken broth fermentation culture was sprayed onto kibbles which were then inoculated with E. coli as described in Table 4.

Treatment 4 of Table 4 was obtained by fermenting a growth media source that was based on beef broth. The beef broth was inoculated with Pediococcus acidilactici, P. pentosaceus, Lactobacillus reuterii, L. acidophilus, Bifidobacterium bifidum, Bacillus coagulans, and B. subtilis at the level of about 1×10⁷ cfu/(gram of beef broth). The inoculated beef broth was placed in a sealed container and then placed in an environment at about 40° C. for about 48 hours to allow it to ferment. Upon fermentation, the beef broth fermentation culture was sprayed onto kibbles which were then inoculated with E. coli as described in Table 4.

Treatment 5 of Table 4 was obtained by fermenting a growth media source that was based on chicken broth. The chicken broth was inoculated with Pediococcus acidilactici, P. pentosaceus, Lactobacillus reuterii, L. acidophilus, Bifidobacterium bifidum, Bacillus coagulans, and B. subtilis at the level of 1×10⁷ cfu/g of chicken broth. The inoculated chicken broth was placed in a sealed container and then placed in an environment at 40° C. for 48 hours to allow it to ferment. Upon fermentation, the chicken broth fermentation culture was sprayed onto kibbles which were then inoculated with E. coli as described in Table 4.

The methods of growing and applying E. coli strains were similar to the ones used in Example 1.

The various test treatments are represented in Table 4.

TABLE 4
Description of Test Treatments Associated with Kibbles

| Treatment # | Treatment Name | Method or Treatment Application |
|---|---|---|
| 1 | Chicken Broth Culture | Fermented chicken broth enriched with Pediococci was |
| | Only (No Kibble | inoculated with E. coli to about 1 × 10⁷ cfu/g and stored |
| | Treated) | at 16 to 22° C. for up to 24 h. |
| 2 | Kibble Treated with | Dry uncoated kibbles were sprayed with a 3% |
| | Pediococci Fermented | application rate of fermented beef broth enriched with |
| | Beef Broth Culture | Pediococci to produce sprayed kibbles that have a |
| | | concentration, by weight, of about 3%. Sprayed |
| | | kibbles were then inoculated with E. coli and stored at |
| | | 20 to 22° C. for up to 72 h. |
| 3 | Kibble Treated with | Dry uncoated kibbles were sprayed with 3% fermented |
| | Pediococci Fermented | chicken broth enriched with Pediococci. Sprayed |
| | Chicken Broth Culture | kibbles were then inoculated with E. coli and stored at |
| | | 20 to 22° C. for up to 72 h. |
| 4 | Kibble Treated with | Dry uncoated kibbles were sprayed with 3% fermented |
| | Pediococci, | beef broth enriched with Pediococci, Lactobacilli, |
| | Lactobacilli, | Bifidobacteria, and Bacilli. Sprayed kibbles were then |
| | Bifidobacteria and | inoculated with E. coli and stored at 20 to 22° C. for up |
| | Bacilli Fermented | to 72 h. |
| | Beef Broth Culture | |
| 5 | Kibble Treated with | Dry uncoated kibbles were sprayed with 3% fermented |
| | Pediococci, | chicken broth previously fermented with Pediococci, |
| | Lactobacilli, | Lactobacilli, Bifidobacteria, and Bacilli. Sprayed |
| | Bifidobacteria, and | kibbles were then inoculated with E. coli and stored at |
| | Bacilli Fermented | 20 to 22° C. for up to 72 h. |
| | Chicken Broth Culture | |

FIG. 8 is a graph 800 showing impact of Pediococci strains on the death of salmonella surrogates in chicken broth and 2% dextrose (Treatment 1). An x-axis 802 represents the length of time (h) the fermentation culture was stored with E. coli. A y-axis 804 represents the level (log₁₀ cfu/g) of Salmonella surrogates (i.e., E. coli). The linear regression line was highly significant (P<0.01).

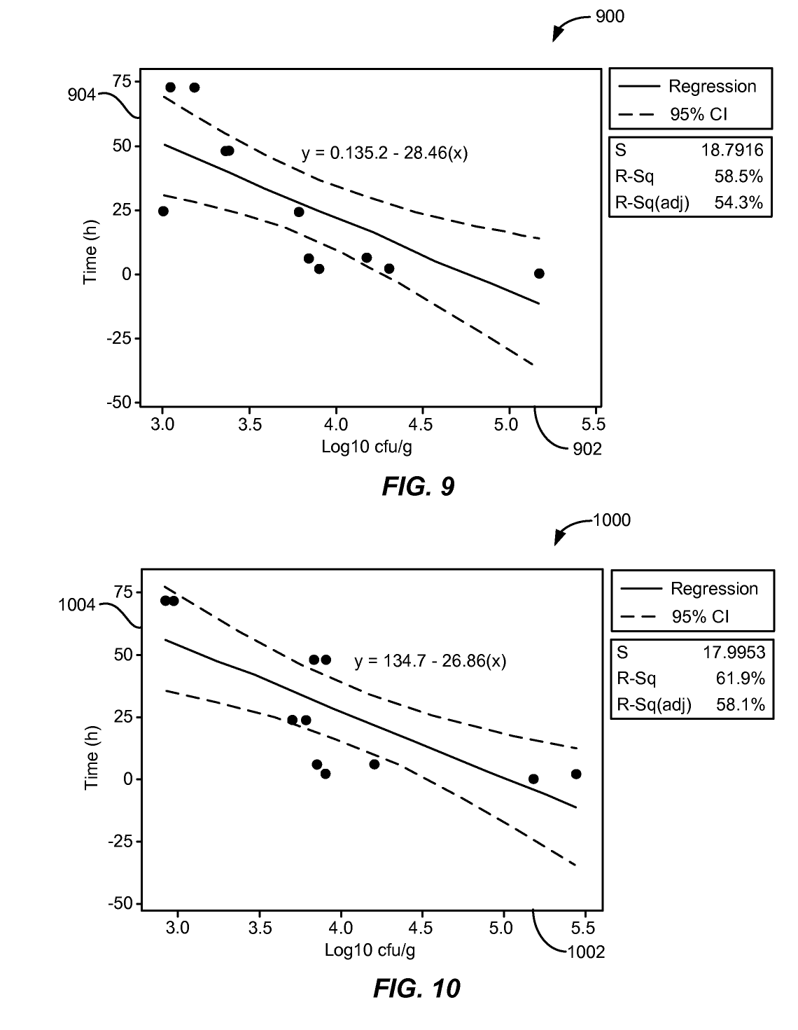

FIG. 9 is a graph 900 showing impact of 3% Pediococci fermented beef broth culture on the death of salmonella surrogates applied to kibbles (Treatment 2). An x-axis 902 represents the level (log₁₀ cfu/g) of Salmonella surrogates (i.e., E. coli). A y-axis 904 represents the length of time (h) the fermentation culture was stored with E. coli. The linear regression line was highly significant (P<0.01).

FIG. 10 is a graph 1000 showing impact of 3% Pediococci fermented chicken broth culture on the death of salmonella surrogates applied to kibbles (Treatment 3). An x-axis 1002 represents the level (log₁₀ cfu/g) of Salmonella surrogates (i.e., E. coli). The linear regression line was highly significant (P<0.01). A y-axis 1004 represents the length of time (h) the fermentation culture was stored with E. coli.

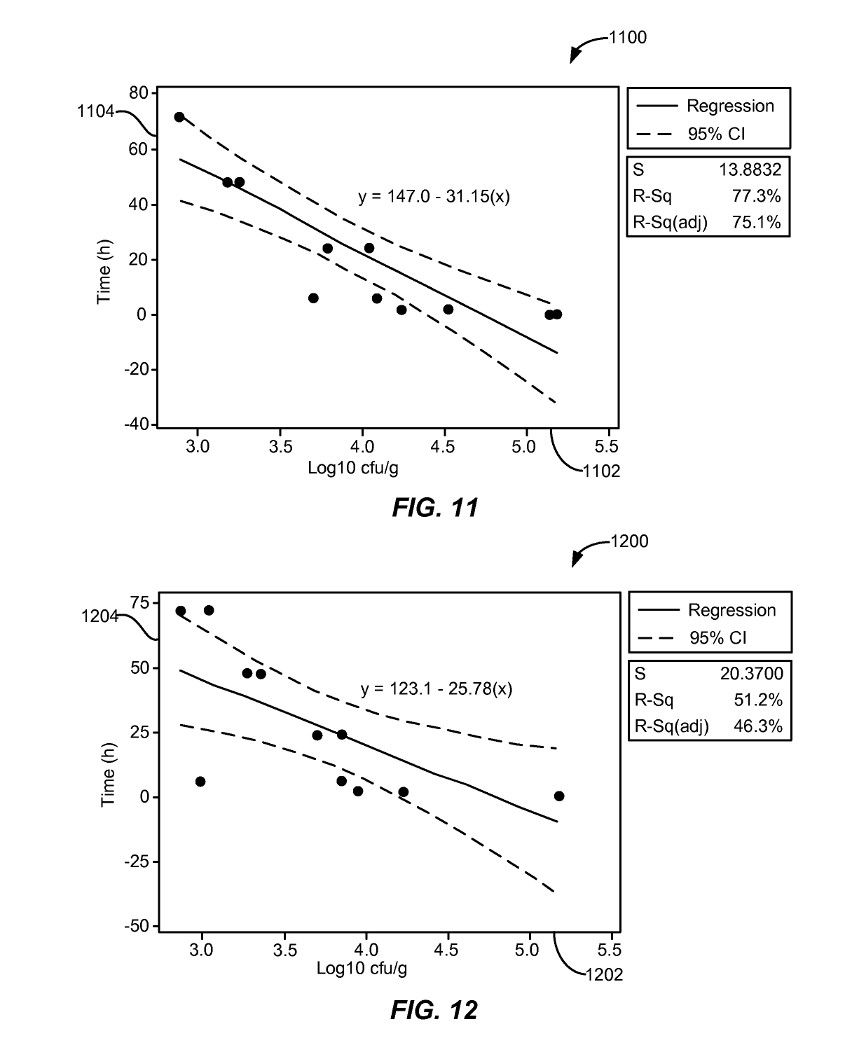

FIG. 11 is a graph 1100 showing impact of 3% Pediococci, Lactobacilli, Bifidobacterium, and Bacilli fermented beef broth culture on the death of salmonella surrogates applied to kibbles (Treatment 4). An x-axis 1002 represents the level (log₁₀ cfu/g) of Salmonella surrogates (i.e., E. coli). A y-axis 1104 represents the length of time (h) the fermentation culture was stored with E. coli. The linear regression line was highly significant (P<0.001).

FIG. 12 is a graph 1200 showing impact of 3% Pediococci, Lactobacilli, Bifidobacterium, and Bacilli fermented chicken broth culture on the death of Salmonella surrogates applied to kibbles (Treatment 5). An x-axis 1202 represents the level (log₁₀ cfu/g) of Salmonella surrogates (i.e., E. coli). A y-axis 1204 represents the length of time (h) the fermentation culture was stored with E. coli. The linear regression line was highly significant (P<0.01).

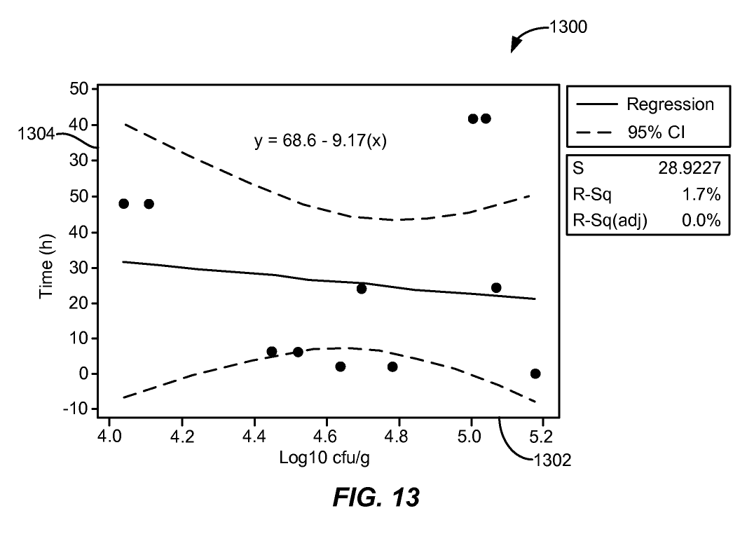

FIG. 13 is a graph 1300 showing impact of not applying a fermented culture to kibbles inoculated with Salmonella surrogates (control). An x-axis 1302 represents the level (log₁₀ cfu/g) of Salmonella surrogates (i.e., E. coli). The linear regression line was not significant (P>0.20). A y-axis 1304 represents the length of time (h) the fermentation culture was stored with E. coli.

Results indicated that Treatment 1, addition of Salmonella surrogates to Chicken broth containing 2% dextrose and Pediococci strains but not applied to kibbles, resulted in 5.48 log₁₀ reduction (p<0.01; adjusted R²=0.86) of the Salmonella surrogates after 24 hours of fermentation (see FIG. 8). These results indicate the ability of Pediococci strains to deactivate Salmonella surrogate strains (i.e., E. coli) either after fermentation or during fermentation results in the destruction of Salmonella surrogates.

Results indicated that Treatment 2, kibbles inoculated with Salmonella surrogates and coated with 3% fermented beef broth containing Pediococci strains, had about a 2.25 log₁₀ reduction of Salmonella surrogates (see FIG. 9; p<0.01; adjusted R2=0.54). Results indicated that Treatment 3, kibbles inoculated with Salmonella surrogates and coated with 3% fermented chicken broth containing Pediococci strains, had about a 2.5 log₁₀ reduction of Salmonella surrogates (see FIG. 10; p<0.01; adjusted R2=0.58).

Results indicated that Treatment 4, kibbles inoculated with Salmonella surrogates and coated with 3% fermented beef broth containing Pediococci, Lactobacilli, Bacterium, and Bacilli strains, had about a 2.5 log₁₀ reduction of Salmonella surrogates (see FIG. 11; p<0.001; adjusted R2=0.75). Results indicated that Treatment 5, kibbles inoculated with Salmonella surrogates and coated with 3% fermented chicken broth containing Pediococci, Lactobacilli, Bacterium, and Bacilli strains, had about a 2.5 log₁₀ reduction of Salmonella surrogates (see FIG. 12; p<0.01; adjusted R2=0.46).

When kibbles were only inoculated with Salmonella surrogates (i.e., E. coli) but not coated with a fermented beef or chicken broth cultures containing various anti-Salmonella strains such as Pediococci, the Salmonella surrogates were not deactivated during a 72 hours time period (see FIG. 13). Thus, the fermentation cultures containing anti-Salmonella strains along with their by-products (e.g., acids, pediocins, bacteriocins, hydrogen peroxide, lactate, etc.) were effective in deactivating the Salmonella surrogates.

### Example 3

### Salmonella Surrogate Inactivation in Broth Using Pediococci Grown at Different Conditions

Applying a topical application that includes Pediococci incubated under varying conditions, according to certain embodiments of the present teachings, to Salmonella surrogates in multiple storage conditions is demonstrated by Example 3.

The Salmonella surrogate organism used in this experiment was E. coli (ATCC strain types BAA 1427, BAA1428, BAA1429, BAA1430, BAA1431). The Pediococci in this experiment served as the bacteria used to create a fermentation culture used to deactivate the E. coli. Prior to assessing their ability to deactivate E. coli, multiple fermentation cultures based on Pediococci were created according to the conditions noted in Table 5.

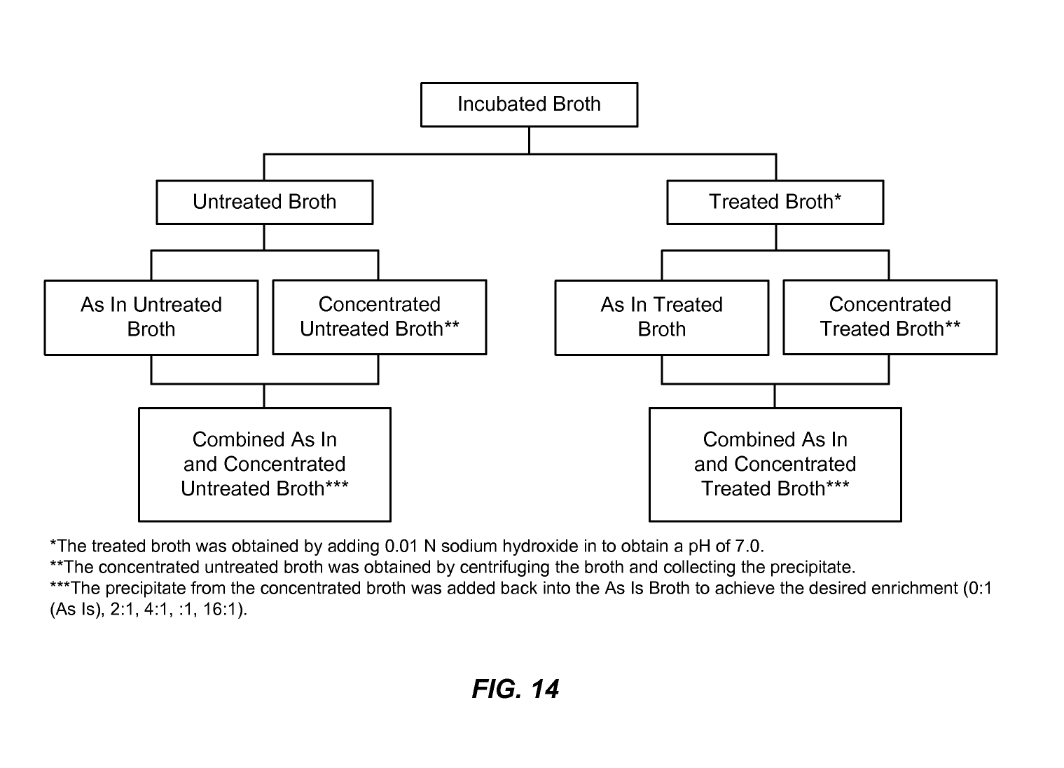

To develop the fermentation culture, the Pediococci starter culture was added to chicken broth (Kroger® Clear Chicken Broth 99% Fat Free, Low Sodium) and supplemented with 2% dextrose (see Table 6). The Pediococci-enriched broth was incubated at 37° C. for 24 h. The resulting incubated broth was used to create the following four different portions: 1) “as is” untreated broth, 2) concentrated untreated broth cells, 3) “as is” treated broth and 4) concentrated treated broth cells (see FIG. 14, which is a flowchart showing certain steps used to generate broth sources for use in Example 3; the amount of “As Is” and Concentrated portions are varied to achieve the desired ratio of “As Is” to Concentrated portions (0:1 (“As Is” Broth), 2:1, 4:1, 8:1 and 16:1).)

Cells were concentrated either 2 times, 4 times, 8 times or 16 times the normal broth level. To create the concentrated untreated cells, a portion of the incubated broth was cooled to 9° C. and then centrifuged for 15 min at 4,000 RPM. The supernatant was decanted and the cells cooled in ice water until being re-suspended (i.e., added back into a portion of the original incubated broth). Another portion of the incubated broth was used to create the treated cells. This portion of the incubated broth was pH adjusted to 7.0 by 0.1N sodium hydroxide to create the treated cells. The treated cells were then used either “as is” or concentrated in the same manner as the untreated cells. The final step in creating the criteria listed in Table 5 was to concentrate the volume necessary to create the intended concentration of the broth.

To then assess the various treatments' relative abilities to deactivate Salmonella surrogates E. coli in solution, 1 ml of E. coli enriched fluid was mixed into 9 ml of each of the treatments noted in Table 5. These mixtures were then stored under a variety of conditions (see Table 7). After the appropriate storage time was completed, the mixture was analyzed for its concentration of E. coli. The analysis technique relied on a cell plate technique using decimal dilutions then plating in Violet Red Bile Agar. The original E. coli inoculum was analyzed and used as the initial challenge count for comparison purposes.

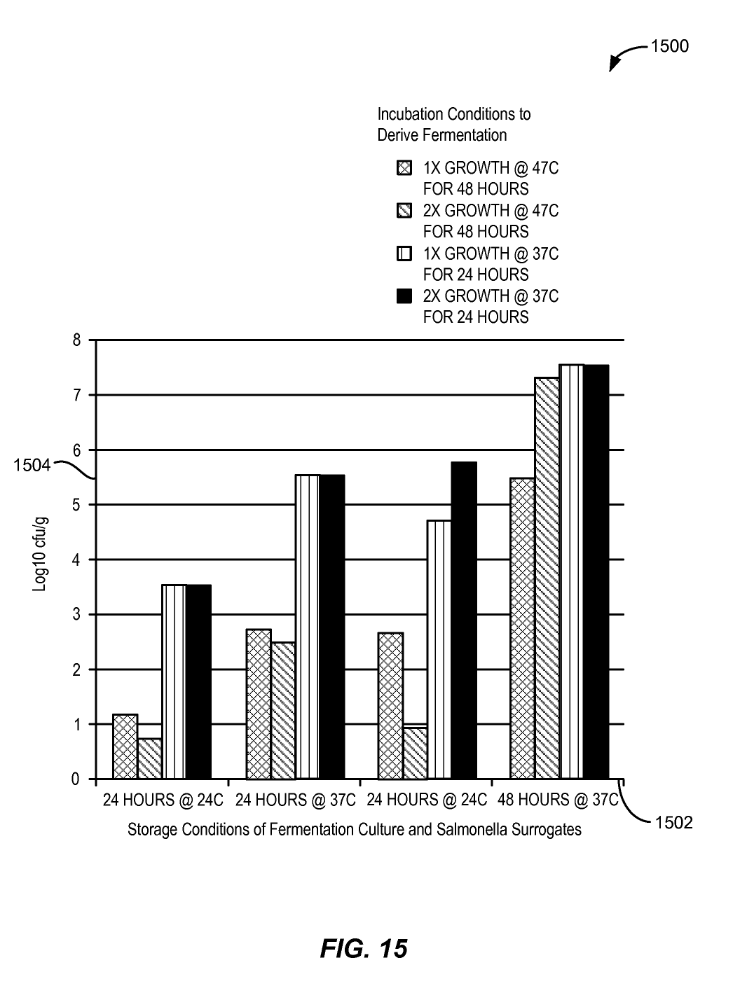

FIG. 15 is a bar graph 1500 showing the growth of salmonella surrogates, incubated under various temperatures and times, in fermentation cultures that were derived through various growth conditions: 1) “as is” incubated broth grown at 47° C. for 48 h, 2) broth grown at 47° C. for 48 hours enriched with twice the concentration untreated Pediococci, 3) “as is” incubated broth is grown at 37° C. for 24 hours and 4) broth grown at 37° C. for 24 hours enriched with twice the concentration. Fermentation cultures were then inoculated with Salmonella surrogates and stored under the following conditions: 1) 24 hours at 24° C., 2) 24 hours at 37° C., 3) 48 hours at 24° C. and 4) 48 hours at 37° C. An x-axis 1502 represents the various Fermentation culture growth conditions and subsequent storage conditions with Salmonella surrogates. A y-axis 1504 represents the level of Salmonella surrogates present (log₁₀ cfu/g).

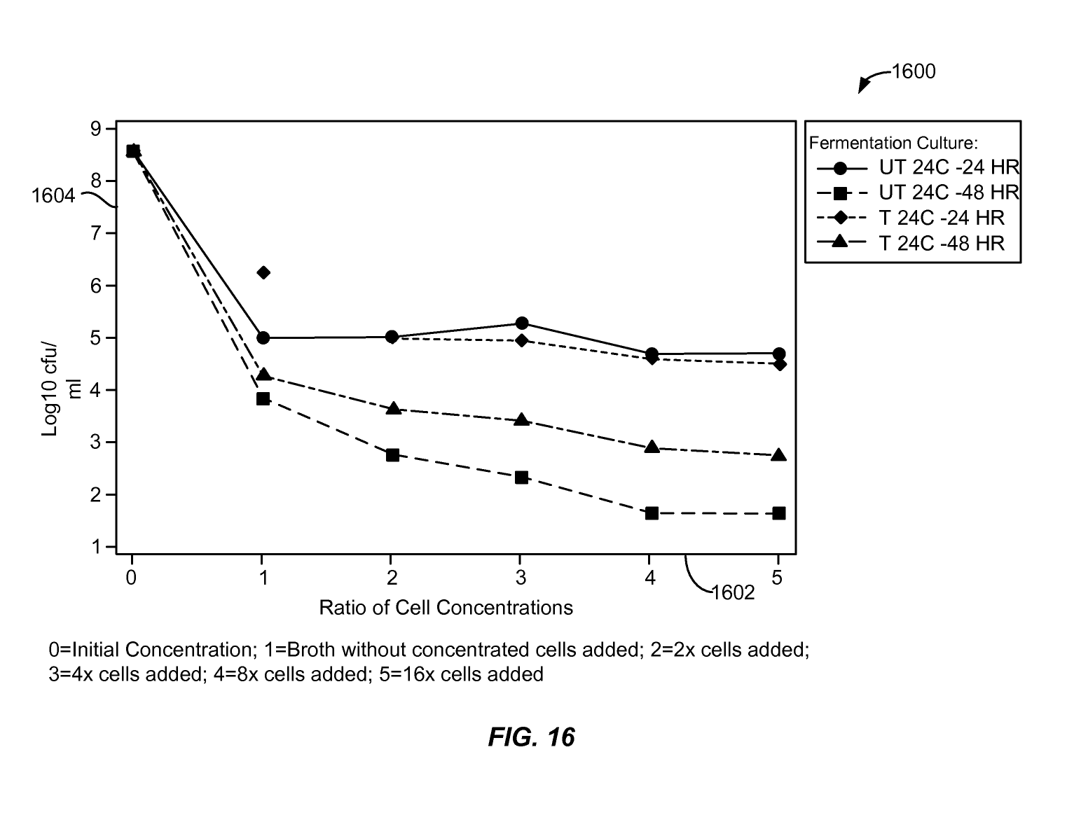

FIG. 16 is a graph 1600 comparing varying concentrations of untreated and treated fermentation cultures' effects on the death of Salmonella surrogates. Fermentation culture sources were derived from the following conditions: 1) untreated broth stored at 24° C. for 24 h, 2) untreated broth stored at 24° C. for 48 h, 3) treated broth stored at 24° C. for 24 hours and the pH adjusted to 7.0 and 4) treated broth stored at 24° C. for 48 hours and the pH adjusted to 7.0. Points on an x-axis 1604 are represented as follows: 0=Initial Concentration; 1=“As Is” Broth; 2=Broth containing twice the concentration of cells; 3=Broth containing four times the concentration of cells; 4=Broth containing eight times the concentration of cells; 5=Broth containing sixteen times the concentration of cells. A y-axis 1602 represents the concentration of Salmonella surrogates (log₁₀ cfu/ml).

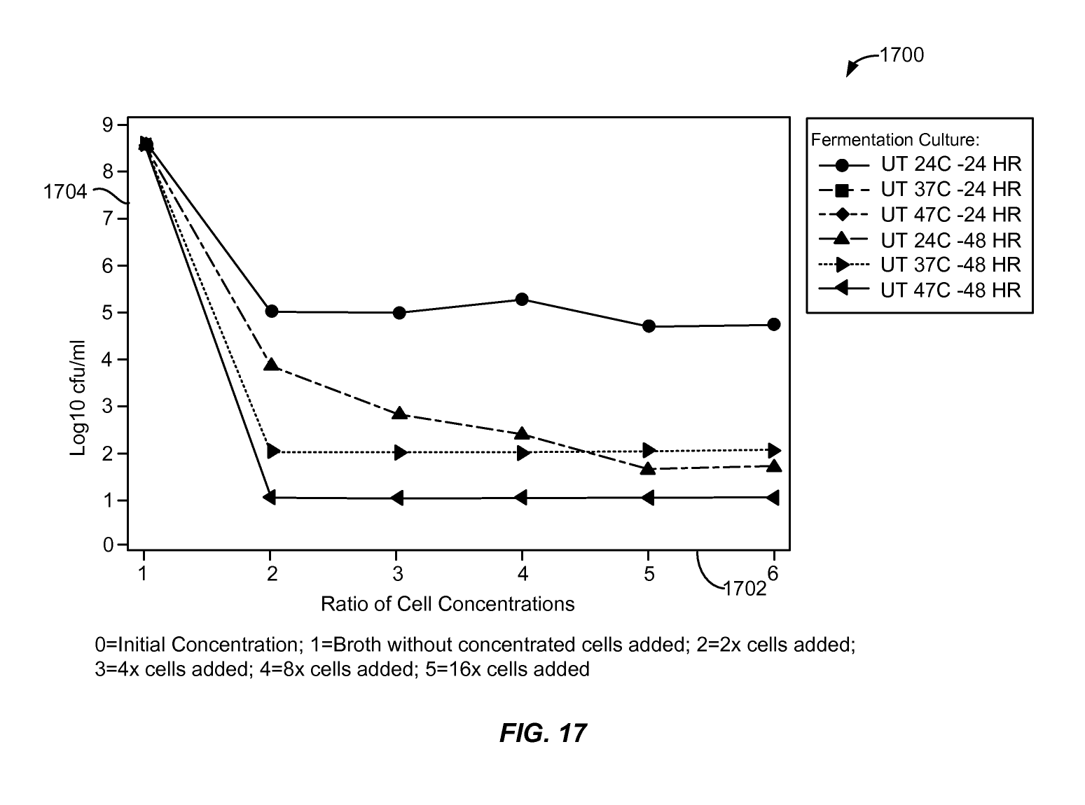

FIG. 17 is a graph 1700 comparing varying concentrations of untreated fermentation cultures stored at varying times and temperatures and their effects on inactivating Salmonella surrogates, i.e., at 24° C., 37° C. and 47° C., for 24 and 48 h. Fermentation cultures were derived from incubating Pediococci at 37° C. for 24 hours and then concentrating cells to the desired levels. Fermentation cultures were then inoculated with E. coli and then stored under the following conditions: 1) 24° C. for 24 h, 2) 37° C. for 24 h, 3) 47° C. for 24 h, 4) 24° C. for 48 h, 5) 37° C. for 48 h, and 6) 47° C. for 48 h. Fermentation cultures were then inoculated with Salmonella surrogates (i.e., E. coli). Points on an x-axis 1502 are represented as follows: 0=Initial Concentration; 1=“As Is” Broth; 2=Broth containing twice the concentration of cells; 3=Broth containing four times the concentration of cells; 4=Broth containing eight times the concentration of cells; 5=Broth containing sixteen times the concentration of cells. A y-axis 1704 represents the concentration of Salmonella surrogates (log₁₀ cfu/ml).

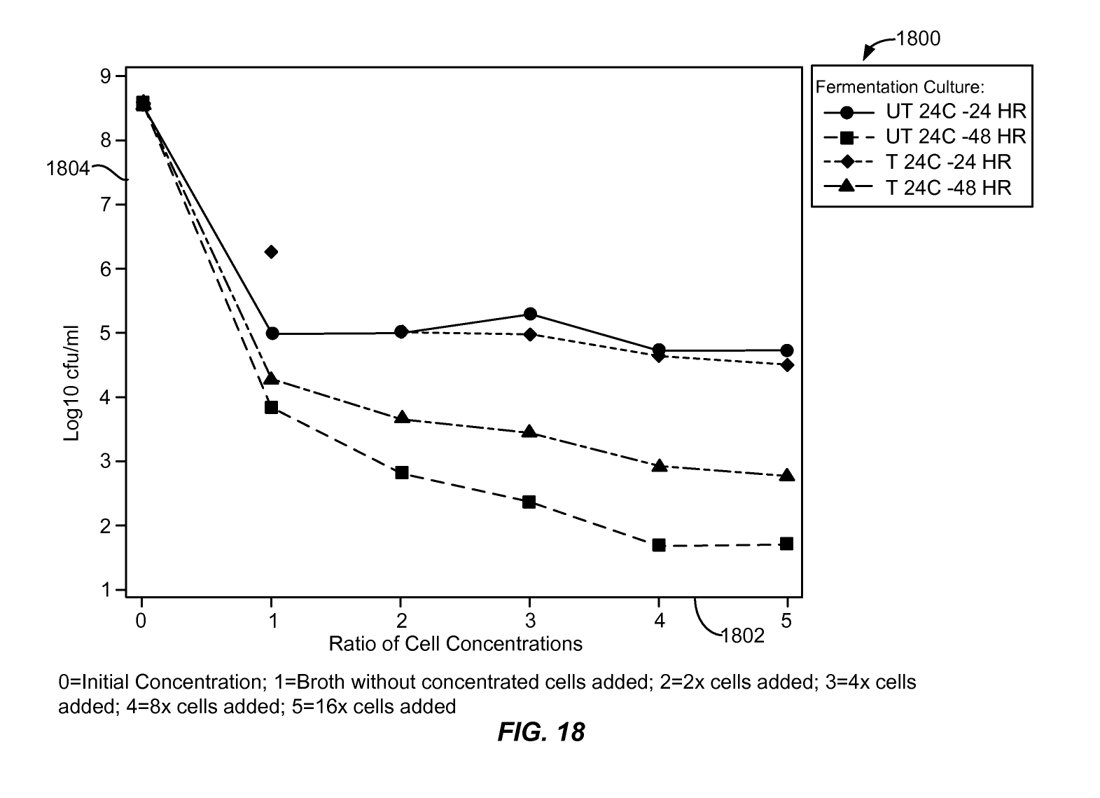

FIG. 18 is a graph 1800 comparing varying concentrations of untreated and treated Fermentation cultures stored at 24° C. for 24 hours and 48 hours on inactivating Salmonella surrogates. Fermentation cultures were derived from incubating Pediococci at 37° C. for 24 hours and then concentrating cells to the desired levels. Fermentation cultures were then inoculated with E. coli and then stored under the following conditions: 1) untreated broth stored at 24° C. for 24 hours; 2) untreated broth stored at 24° C. for 48 hours; 3) treated broth stored at 24° C. for 24 h; and 4) treated broth stored at 24° C. for 48 hours. Points on an x-axis 1802 are represented as follows: 0=Initial Concentration; 1=“As Is” Broth; 2=Broth containing twice the concentration of cells; 3=Broth containing four times the concentration of cells; 4=Broth containing eight times the concentration of cells; 5=Broth containing sixteen times the concentration of cells. A y-axis 1804 represents the concentration of Salmonella surrogates (log₁₀ cfu/ml).

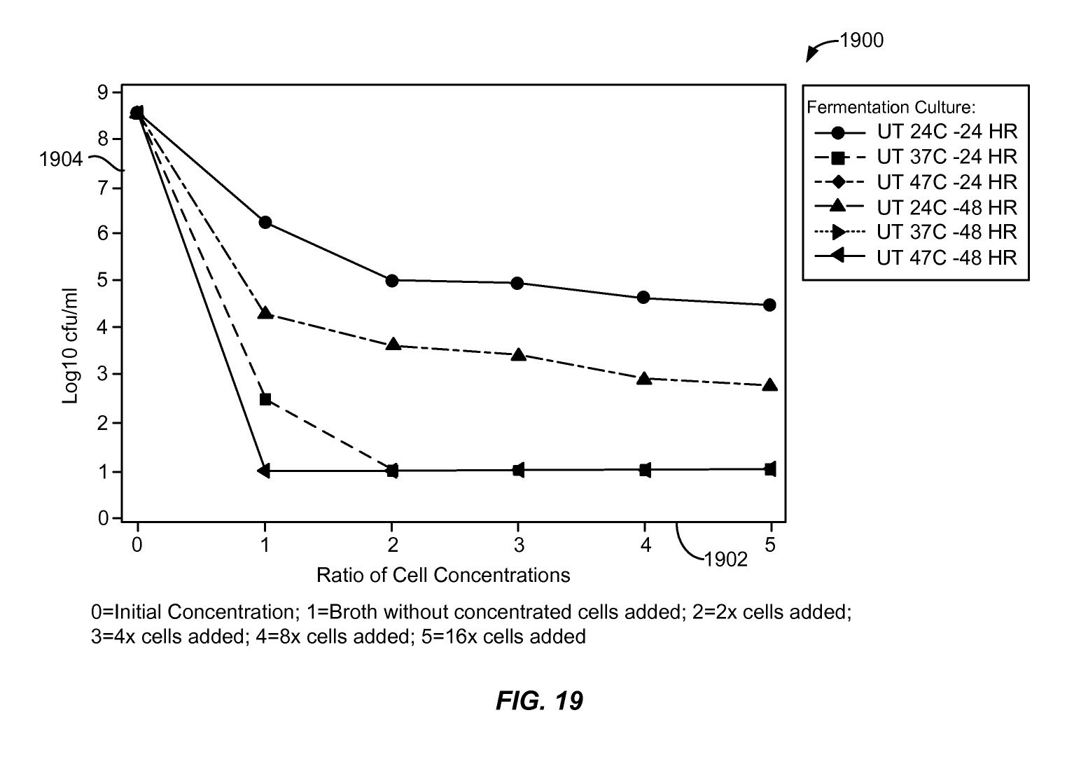

FIG. 19 is a graph 1900 comparing varying concentrations of treated fermentation cultures Stored at 24° C., 37° C. and 47° C. for 24 hours and 48 hours on inactivating Salmonella surrogates. Fermentation cultures were derived from incubating Pediococci at 37° C. for 24 hours and then concentrating cells to the desired levels. Fermentation cultures were then inoculated with E. coli and stored under the following conditions: 1) treated broth stored at 24° C. for 24 hours; 2) treated broth stored at 37° C. for 24 h; 3) treated broth stored at 47° C. for 24 hours; 4) treated broth stored at 24° C. for 48 hours; 5) treated broth stored at 37° C. for 48 hours; and 6) treated broth stored at 47° C. for 48 hours. Points on an x-axis 1902 are represented as follows: 0=Initial Concentration; 1=“As Is” Broth; 2=Broth containing twice the concentration of cells; 3=Broth containing four times the concentration of cells; 4=Broth containing eight times the concentration of cells; 5=Broth containing sixteen times the concentration of cells. A y-axis 1904 represents the concentration of Salmonella surrogates (log₁₀ cfu/ml).

Results indicated that a greater length of time incubating E. coli in the presence of the Pediococci-enriched fermentation cultures (48 hours more than 24 h) is associated with a greater amount of death of E. coli (see FIG. 15). Results further indicated that a temperature of 37° C. rather than 24° C. was more effective at deactivating E. coli. Results also indicated that regardless of being treated or concentrated, Pediococci-enriched fermentation cultures derived from incubation at 37° C. rather than from incubation at 47° C. were more effective at deactivating E. coli.

Further, as shown below with reference to Example 12, the present teachings recognize that incubation at 30° C. is even more effective at deactivating E. coli. Thus, incubation at 30° C. represents a preferred embodiment of the present teachings.

When results from treated and untreated Pediococci incubated broth were compared, no advantage was apparent from the treatment of broth (adjusting to pH 7.0) regardless of the level of broth concentration (see FIG. 16). When results from “as is” (unconcentrated) Pediococci broth were compared to concentrated Pediococci broth, the concentrated broth was more effective in deactivating E. coli, especially when the broth containing E. coli was stored at 24° C. (see FIG. 17). Further, eight times the concentration of Pediococci broth was optimal for deactivating E. coli. These results taken with those represented in FIG. 16 indicate that the factor that deactivates E. coli is closely associated with the Pediococci cells themselves, and thus the lactic acid is less critical in E. coli death than previously believed. Thus, according to certain embodiments of the present teachings, because the factor that deactivates E. coli is associated with the Pediococci cells themselves, dead or dormant Pediococci cells are used in a topical application to inhibit pathogen activity on a food surface.

When broth containing Pediococci and E. coli was stored at 24° C. a reduced amount of E. coli death occurred compared to broth stored at 37° C. and 47° C. (see FIGS. 17, 18 and 19). Results from broth stored at 24° C. also indicated that the optimal concentration for death of E. coli occurred at eight times or greater concentration of Pediococci.

TABLE 5
Description of Criteria Under Which Pediococci Cells Were Incubated

| | | Volume of | |
|---|---|---|---|
| | | Broth Untreated | |
| | | Cells were Obtained | Treated Cells |
| | | from and Added | Added Back Into |
| | Original | Back Into Original | Original |
| | Incubated Broth, | Incubated Broth, | Incubated |
| Treatment # | ml | ml | Broth |
| 1 | 0 | 0 | 0 |
| 2 | 100 | 0 | 0 |
| 3 | 100 | 200 | 0 |
| 4 | 100 | 400 | 0 |
| 5 | 100 | 800 | 0 |
| 6 | 100 | 1,600 | 0 |
| 7 | 100 | 0 | 0 |
| 8 | 100 | 0 | 200 |
| 9 | 100 | 0 | 400 |
| 10 | 100 | 0 | 800 |
| 11 | 100 | 0 | 1,600 |

TABLE 6
Formula for Base Growth of Pediococci

| Ingredient | Percent by Weight |
|---|---|
| Chicken Broth | To 100 |
| Dextrose | 2 |
| Starter Culture* | 0.0626 |

*Starter Culture contains sufficient Pediococcus acidilactici and Pediococcus pentosaceus to provide 1 × 10⁷ cfu/(ml of chicken broth). The concentration, 0.0626%, is an approximate concentration. Since the concentration of bacteria in the starter culture can vary, the actual percentage included in the Fermented Mixture may vary.

TABLE 7
Storage Conditions for E. coli in the Presence of Pediococci

| Condition ID | Temperature, ° C. | Time, h |
|---|---|---|
| A | 24 | 24 |
| B | 24 | 48 |
| C | 37 | 24 |
| D | 37 | 48 |

### Example 4

### Application of a Fermentation Culture to Deactivate Salmonella Surrogates on the Surface of Kibbles

Applying a topical application in a fermentation culture to reduce pathogenic activity on food kibbles, according to one embodiment of the present teachings, is shown by Example 4.

The methods of growing and applying E. coli strains were similar to the ones used in Example 1.

Chicken broth was inoculated with Pediococcus acidilactici and P. pentosaceus at the level of 1×10⁷ cfu/g of chicken broth and enriched with 2% dextrose as a culture energy source. The inoculated chicken broth was placed in a sealed container and then placed in an environment at 37° C. for 24 hours to allow it to ferment. The fermented broth was then sprayed onto two sets of kibbles that were made by condition 1 (first inoculated with salmonella surrogates and then sprayed with fermented chicken broth) or condition 2 (first sprayed with fermented chicken broth and then inoculated with salmonella surrogates). The first set of kibbles were inoculated with a concentrated spray of salmonella surrogates and dried for 24 hours. The inoculated kibbles were then sprayed with an 8:1 (similar to treatment 5 in Table 5) concentrate of the fermented chicken broth (3% w/w). A second set of kibbles was sprayed with the fermented broth and dried for 24 hours. These sprayed kibbles were then inoculated with an 8:1 concentrated spray of salmonella surrogates. After applying the fermented chicken broth and salmonella surrogates to kibbles, the kibbles were stored at the following temperature options: 1) 24° C., 2) 37° C. and 3) 37° C. for 4 hours followed by 24° C. for the remaining time. Note that the third option (37° C. for 4 hours followed by 24° C. for the remaining time was only done using the first set of kibbles (inoculated with salmonella surrogates then sprayed with fermented broth). Kibbles were analyzed for E. coli levels prior to (time 0) and 24, 48 and 72 hours after storage.

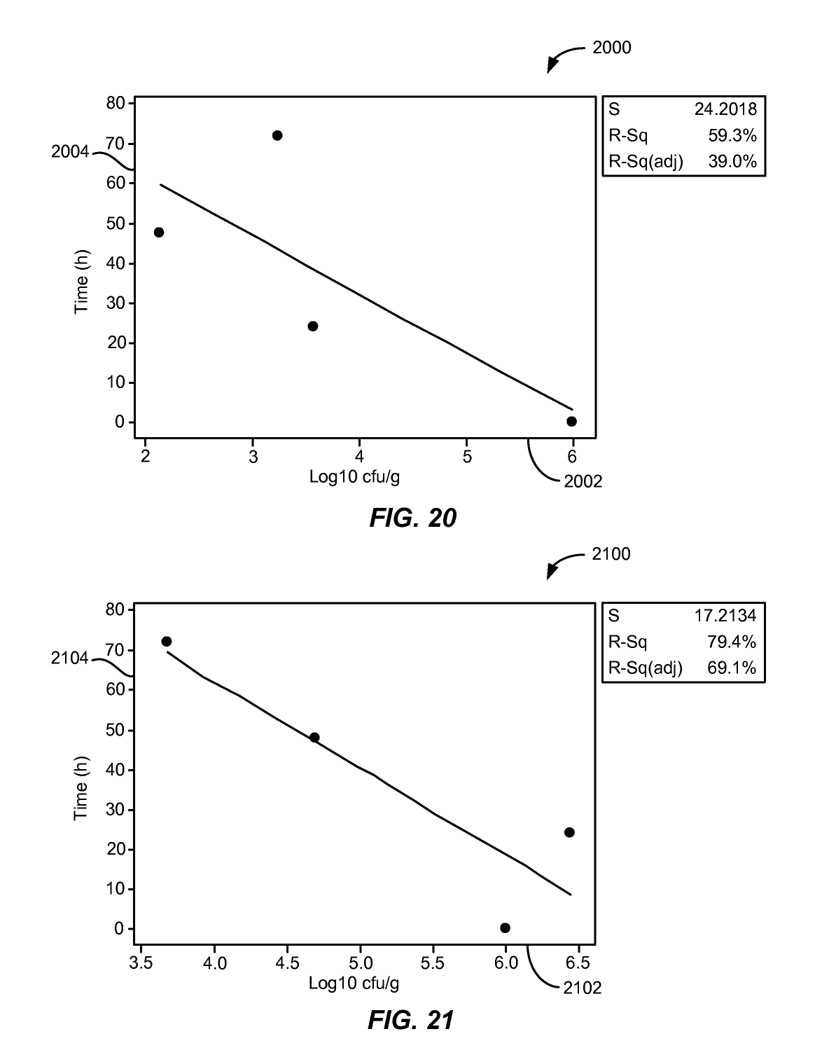

FIG. 20 is a line graph 2000 showing the impact of 3% (w/w) Pediococci fermented chicken broth culture on the inactivation of Salmonella surrogates applied to coated kibbles when stored at 24° C. Samples were first inoculated with Salmonella surrogates and then sprayed with fermented chicken broth. An x-axis 2002 represents the level (log₁₀ cfu/g) of Salmonella surrogates (i.e., E. coli). A y-axis 2004 represents the length of time (h) the fermentation culture was stored with E. coli.

FIG. 21 is a line graph 2100 showing impact of 3% (w/w) Pediococci fermented chicken broth culture on the inactivation of Salmonella surrogates applied to coated kibbles when stored at 24° C. Samples were first sprayed with fermented chicken broth and then inoculated with Salmonella surrogates. An x-axis 2102 represents the level (log₁₀ cfu/g) of Salmonella surrogates (i.e., E. coli). A y-axis 2104 represents the length of time (h) the fermentation culture was stored with E. coli.

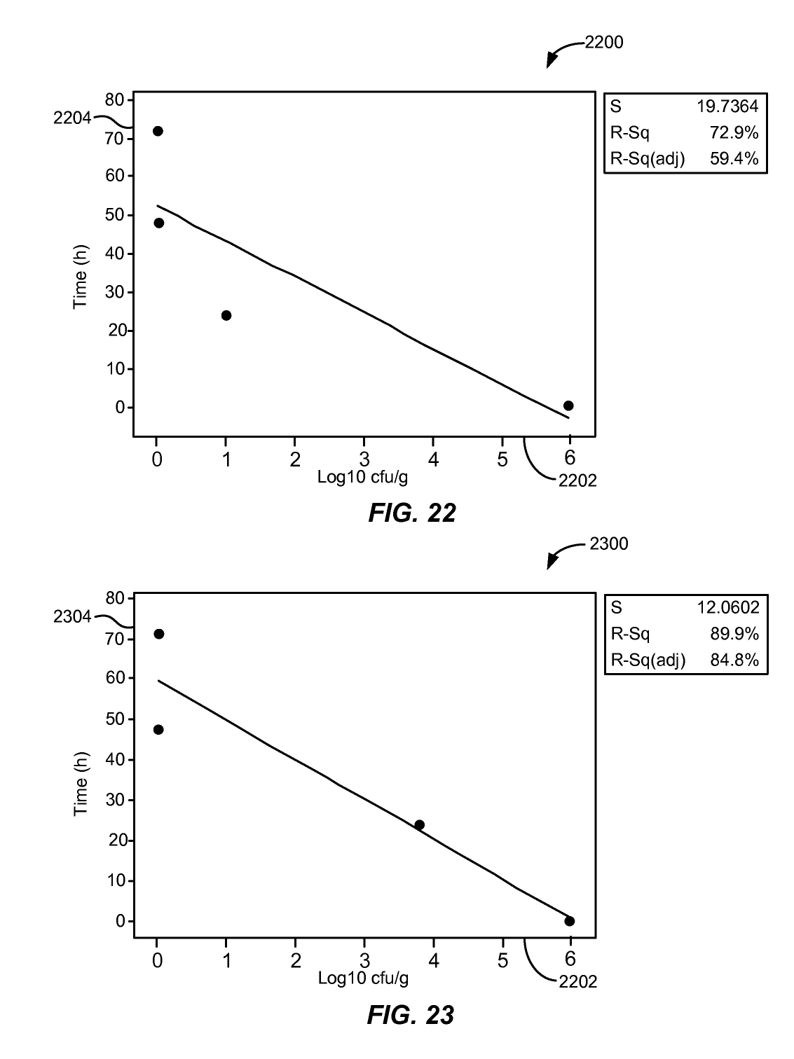

FIG. 22 is a line graph 2200 showing the impact of 3% (w/w) Pediococci fermented chicken broth culture on the inactivation of Salmonella surrogates applied to coated kibbles when stored at 37° C. Samples were first inoculated with Salmonella surrogates and then sprayed with fermented chicken broth. An x-axis 2202 represents the level (log₁₀ cfu/g) of Salmonella surrogates (i.e., E. coli). A y-axis 2204 represents the length of time (h) the fermentation culture was stored with E. coli.

FIG. 23 is a line graph 2300 showing the impact of 3% (w/w) Pediococci fermented chicken broth culture on the inactivation of Salmonella surrogates applied to coated kibbles when stored at 37° C. The samples were first sprayed with fermented chicken broth and then inoculated with Salmonella surrogates. An x-axis 2302 represents the level (log₁₀ cfu/g) of Salmonella surrogates (i.e., E. coli). A y-axis 2304 represents the length of time (h) the fermentation culture was stored with E. coli.

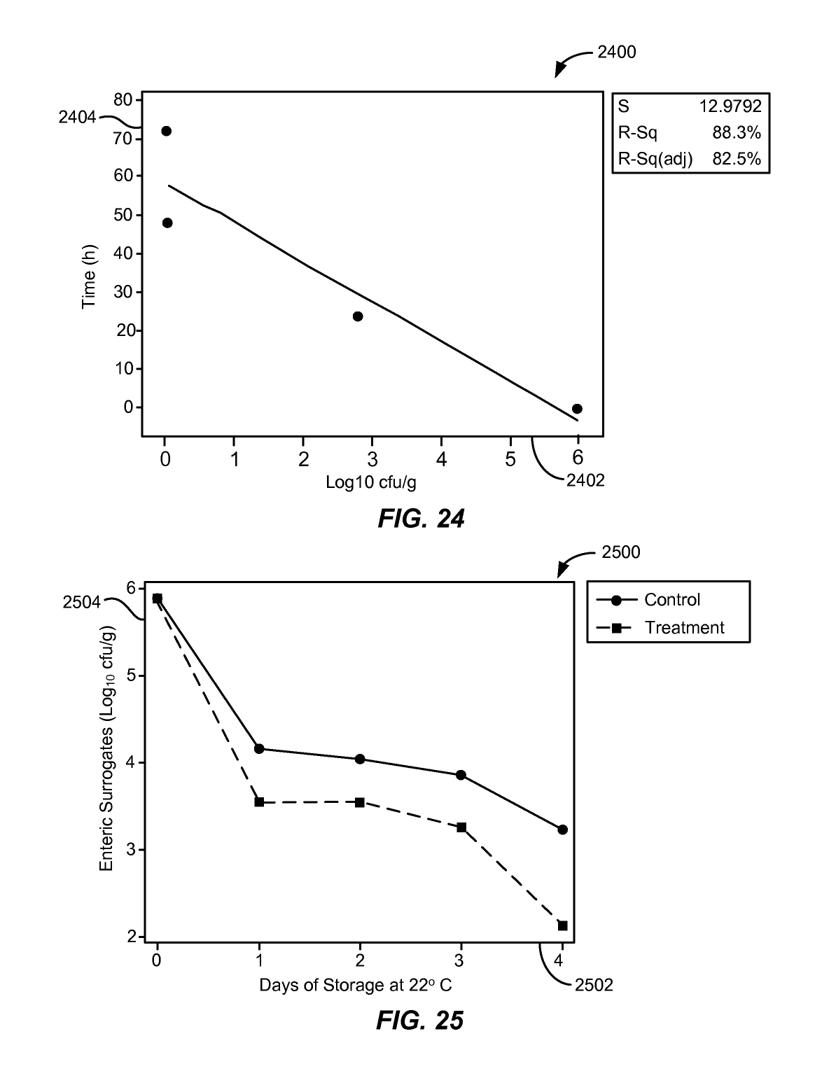

FIG. 24 is a line graph 2400 showing the impact of 3% (w/w) Pediococci fermented chicken broth culture on the inactivation of Salmonella surrogates applied to coated Kibbles when stored at 37° C. for 4 hours followed by 24° C.: Samples were first inoculated with Salmonella surrogates and then sprayed with fermented chicken broth. An x-axis 2402 represents the level (log₁₀ cfu/g) of Salmonella surrogates (i.e., E. coli). A y-axis 2404 represents the length of time (h) the fermentation culture was stored with E. coli.

Results for each temperature condition are reported in FIGS. 20-24. Also reported are the two different conditions by which the kibbles were inoculated with salmonella surrogates and sprayed with fermented chicken broth. Regardless of which condition was used to inoculate and spray the kibbles, storage at 24° C. resulted in almost a four log₁₀ reduction of Salmonella surrogates (see FIGS. 20 and 21). Regardless of which condition was used to inoculate and spray the kibbles, storage at 37° C. resulted in a six log₁₀ reduction of Salmonella surrogates by 48 hours (see FIGS. 22 and 23). Similarly storage at 37° C. for 4 hours followed by storage at 24° C. for up to 72 hours also resulted in a six log₁₀ reduction of Salmonella surrogates by 48 hours (see FIG. 24). The two phase temperature condition (37° C. for 4 hours followed by 24° C.) was chosen as being generally indicative of the cooling phase conditions after kibbles are coated and before they are packaged.

### Example 5

### Freeze-Dried Process of Storage of Topical Application

A process for storing a topical application by freeze drying, according to one embodiment of the present teachings, is shown by Example 5.

In this example, a freeze-dried embodiment of storage of a topical application is demonstrated.

The method of growing Pediococci is similar to what was shown in Example 3 for the concentration of cells eight times the normal broth level.

After fermenting the broth for 24 h, the fermented broth is freeze dried to less than 12% moisture. The freeze-dried fermented broth is then packaged in sealed containers and stored at less than 49° C. for 12 months.

### Example 6

### Rejuvenation of Freeze-Dried Topical Application Applied to Kibbled Pet Food

A process for rejuvenating a freeze-dried topical application, according to one embodiment of the present teachings, is shown by Example 6.

The freeze dried topical application of Example 5 is emptied from the sealed container and placed into 21° C. distilled water for 30 minutes to rejuvenate the bacteria.

After the topical application has been rejuvenated it is applied on the surface of kibbled food (e.g., as demonstrated in treatment 3 of Example 2).

### Example 7

### Refrigerated Process of Storage of Topical Application

A process for storing a topical application by refrigeration, according to one embodiment of the present teachings, is shown by Example 7.

The method of growing Pediococci is similar to what was shown in Example 3 for the concentration of cells eight times the normal broth level.

After fermenting the broth for 24 hours the fermented broth is packaged into sealed containers and refrigerated at 4° C.

### Example 8

### Use of Refrigerated Topical Application Applied to Kibbled Pet Food

A process for using a topical application that was stored by refrigeration for decontaminating kibbled pet food, according to one embodiment of the present teachings, is shown by Example 8.

The refrigerated topical application of Example 7 is allowed to warm up to at least 16° C.

After the topical application has warmed up to at least 16° C., it is applied on the surface of kibbled pet food, as shown in treatment 3 of Example 2.

### Example 9

### Process of Freezing and Storage of a Topical Application

A process for freezing and storing a topical application, according to one embodiment of the present teachings, is shown by Example 9.

The method of growing Pediococci is similar to what was demonstrated in Example 3 for the concentration of cells eight times the normal broth level.

After fermenting the broth for 24 hours the fermented broth is packaged into sealed containers and frozen at −11° C.

### Example 10

### Use of Frozen Topical Application Applied to Kibbled Pet Food

A process for preparing a frozen topical application for decontaminating a food product, according to one embodiment of the present teachings, is shown by Example 10.

The frozen topical application of Example 9 is allowed to warm up to at least 16° C.

After the topical application has warmed up to at least 16° C. it is applied on the surface of kibbled food as demonstrated in treatment three of Example 2.

### Example 11

### Fines and Broken Pieces in Production of Food Pieces

A process for using fines and broken food pieces, that were coated with a topical application in a first food production, in a subsequent food production, according to one embodiment of the present teachings, is shown by Example 11.

The topical application was created similar to the chicken broth with Pediococci strains as noted in Example 4. The topical application was coated onto dog food kibbles. The dog food kibbles were extruded and then dried to six percent moisture. Then the kibbles were placed into a mistcoater (APEC®) where the topical application was applied at 1.5% (w/w).

After the kibbles left the mistcoater, they passed over a screen to allow the fines to be removed from the kibbles. The fines contain small amounts of topical application. The surfaces of the fines residue areas and collection tanks become coated with the topical application. After sufficient accumulation, the fines are re-worked into ingredient mixtures for future production runs of kibbles. The interior of these future kibbles now contains the topical application including lactic acid, bacteriocins and other substances detrimental to the growth of pathogens, including salmonella.

### Example 12

### Salmonella Surrogate Inactivation in Broth Using Pediococci Grown at Different Temperatures

According to the embodiment of Example 12, anti-pathogenic activity of Pediococci samples incubated in broth at different temperatures and then tested in the presence of Salmonella Surrogates is shown.

The Salmonella surrogate organisms used in this experiment were strains of E. coli (ATCC strain types BAA 1427, BAA1428, BAA1429, BAA1430, BAA 1431). Pediococcus acidilactici and Pediococcus pentosaceus served as the bacteria sources used to create a fermentation culture that was used to deactivate the E. coli.

To develop the fermentation cultures, the Pediococci starter culture was added to low sodium chicken broth (Kroger® Clear Chicken Broth 99% Fat Free, Low Sodium) and supplemented with 2% dextrose (see Table 8). Multiple samples of the Pediococci-enriched broth were incubated at 20° C., 30° C. or 37° C. for 24 hours. The resulting incubated broth was used to create the following two different portions: 1) “as is” incubated broth and 2) concentrated incubated broth cells. Cells were concentrated at eight times the normal broth level (i.e., 8:1) for the cultures incubated at 30° C. and 37° C. To create the concentrated cells, a portion of the incubated broth was cooled to 9° C. and then centrifuged for 15 min at 4,000 RPM. The supernatant was decanted and the remaining solid residue constituted cells that were then cooled in ice water until being re-suspended (i.e., added back into a portion of the original incubated broth). The final step in creating the 30° C. and 37° C. concentrated incubated cultures was to add the solid residue collected from eight volumes of concentrated incubated broth cells back to one volume of the “as is” incubated broth to create the 8:1 concentration samples.

The 20° C. incubation resulted in slower cell growth and as a result the density of cells contained in the “as is” incubated broth was lower than broth grown at 30° C. and 37° C. (there was little difference in cell density between the 30° C. and 37° C. incubated broth). As such, the final step in creating the 20° C. incubated culture was to add in additional concentrated cells obtained from the 20° C. incubation to assure that a similar cell concentration existed between the 20° C. concentrated incubated broth and the 30° C. and 37° C. concentrated incubated broth. The method used to assess cell density in the incubated broth involved using a MacFarland modified standard.

To then assess the various treatments' abilities to deactivate Salmonella surrogates E. coli in solution, 1 ml of E. coli enriched fluid was mixed into 9 ml samples of each of the 20° C., 30° C. and 37° C. concentrated incubated broth. These mixtures were then stored in an incubator set at 37° C. for 24 hour. After the appropriate storage time was completed, the mixture was analyzed for its concentration of E. coli. The analysis technique relied on a cell plate technique using decimal dilutions that were plated onto Violet Red Bile Agar and counted.

Results indicated that 30° C. concentrated incubated broth performed as well as 37° C. concentrated incubated broth in killing E. coli (see Table 9). Results further suggest that 20° C. concentrated incubated broth was as effective in killing E. coli as 30° C. and 37° C. concentrated incubated broth (see Table 9). However, comparison of the results from the 20° C. concentrated incubated broth to the 30° C. or 37° C. concentrated incubated broth is somewhat complicated by the slower growth rate of cells at 20° C. and the subsequent need to further adjust the concentration of the 20° C. incubated broth.

TABLE 8
Formula for Base Growth of Pediococci

| Ingredient | Percent by Weight |
|---|---|
| Chicken Broth | To 100 |
| Dextrose | 2 |
| Starter Culture* | 0.0626 |

*Starter Culture contains sufficient Pediococcus acidilactici and Pediococcus pentosaceus to provide 1 × 10⁷ cfu/ml of chicken broth. The concentration, 0.0626%, is an approximate concentration. Since the concentration of bacteria in the starter culture can vary, the actual percentage included in the Fermented Mixture may vary.

TABLE 9
Impact of Incubation Temperature for Deactivating E. coli

| | E. coli level | |
|---|---|---|
| | Before Incubation | E. coli level After Incubation |
| Treatment | (cfu/ml) | (cfu/ml) |
| 37° C. Concentrated | 3.71 × 10⁸ | 1.10 × 10⁴ |
| Incubated Broth | | |
| 30° C. Concentrated | 3.71 × 10⁸ | 6.03 × 10² |
| Incubated Broth | | |
| 20° C. Concentrated | 3.71 × 10⁸ | 3.63 × 10² |
| Incubated Broth | | |

### Example 13

### Use of Bacteria to Create a Gravy for a Shelf-Stable Beef and Vegetable Mixture

According to the embodiment of Example 13, Pediococci are added to preserve a gravy for use in a beef and vegetables mixture that is shelf-stable.

The gravy was prepared by the formula represented in Table 10. Beef broth, flour, and dextrose were combined and then heated to boiling (100° C.) for one min. This mixture resulted in the formation of a gravy. This mixture was then cooled to about 49° C. and then the pediococci culture (Pediococcus acidilactici and Pediococcus pentosaceus) was added. The culture was mixed into the mixture and then allowed to incubate at 37° C. for 48 hours. Upon 48 hours incubation the gravy mixture's pH was 3.5.

TABLE 10
Gravy Formula

| Ingredient | Amount, g |
|---|---|
| Beef Broth | 900 |
| Whole Wheat Flour | 50 |
| Dextrose | 18 |
| Starter Culture* | 1 |

*Starter Culture contains sufficient Pediococcus acidilactici and Pediococcus pentosaceus to provide 1 × 10⁷ cfu/ml of gravy.

### Example 14

### Creating a Shelf-Stable Beef and Vegetables Mixture

According to the embodiments of Example 8, a shelf-stable beef and vegetable gravy mixture was created.

Beef and vegetables was prepared by the formula represented in Table 11. Ground beef (20% fat, 80% lean) was placed in a Food Saver® bag and the bag was sealed. The beef in the sealed bag was then placed into an 80° C. water bath to achieve a meat cook temperature of at least 71° C. for ten min. The meat was cooked at least 74° C. for ten min. The final temperature of the meat was measured at 79° C. After cooking the meat, the food saver bag containing the meat was opened and the peas and sliced carrots were added to the meat. Starter culture (Pediococcus acidilactici and Pediococcus pentosaceus) was mixed into the meat, peas and carrots. Finally, the gravy from Example 1 was added to the entire mixture of meat, peas, carrots and starter culture. This complete mixture was placed into Zip-Lock® tubs and sealed with Zip-Lock® covers. Several tubs were placed in incubation at 20° C. to 22° C. or 37° C.

Weekly assessments for product stability were carried out and are reported in Table 12. As noted, at all time points assessed, the color, odor, gas, visible growth and pH has not changed since Time 0.

TABLE 11
Beef, Vegetables and Gravy Formula

| Ingredient | Amount, g |
|---|---|
| Ground Beef (20% fat, 80% lean) | 900 |
| Peas (canned, water drained) | 200 |
| Carrots (canned, sliced, water drained) | 200 |
| Starter Culture* | 1 |
| Gravy | 900 |

*Starter Culture contains sufficient Pediococcus acidilactici and Pediococcus pentosaceus to provide 1 × 10⁷ cfu/g of beef and vegetables.

TABLE 12
Beef, Vegetables and Gravy Formula Stability Results

| Time | pH | Color | Odor | Gas | Visible Growth |
|---|---|---|---|---|---|
| 0 | 4.0 | Normal | Good | None | None |
| 1 | 4.0 | Normal | Good | None | None |
| 2 | 4.0 | Normal | Good | None | None |
| 3 | 4.0 | Normal | Good | None | None |
| 4 | 4.0 | Normal | Good | None | None |
| 5 | 4.0 | Normal | Good | None | None |

### Example 15

### Use of Bacteria to Create a Gravy for a Chicken and Vegetables Mixture

According to the embodiment of example 15, pediococci are added to preserve a gravy for use in a chicken and vegetables mixture.

The gravy was prepared by the formula represented in Table 13. Chicken broth, flour, and dextrose were combined and then heated to boiling (100° C.) for one minute. This mixture resulted in the formation of gravy. This mixture was then cooled to about 49° C. and then the pediococci culture (which included Pediococcus acidilactici and Pediococcus pentosaceus) was added. The culture was mixed into the mixture and then allowed to incubate at 37° C. for 48 h.

Upon 48 hours incubation the gravy mixture's pH was 3.4.

TABLE 13
Gravy Formula

| Ingredient | Amount, g |
|---|---|
| Chicken Broth | 900 |
| Whole Wheat Flour | 50 |
| Dextrose | 18 |
| Starter Culture* | 1 |

*Starter Culture contains sufficient Pediococcus acidilactici and Pediococcus pentosaceus to provide 1 × 10⁷ cfu/ml of gravy.

### Example 16

### Creating a Shelf-Stable Chicken and Vegetables Mixture

According to the embodiment of Example 16, a chicken, vegetables and gravy mixture was created.

Chicken and vegetables were prepared by the formula represented in Table 14. Chicken was placed in a Food Saver® bag and the bag was sealed. The chicken in the sealed bag was then placed into an 80° C. water bath to achieve a meat cook temperature of at least 71° C. for ten min. The meat was cooked at least 74° C. for ten min. The final temperature of the meat was measured at 79° C. After cooking the meat, the food saver bag containing the meat was opened and the peas and sliced carrots were added to the meat. Starter culture (Pediococcus acidilactici and Pediococcus pentosaceus) was mixed into the meat, peas and carrots. Finally, the gravy from Example 9 was added to the entire mixture of meat, peas, carrots and starter culture. This complete mixture was placed into Zip-Lock® tubs and sealed with Zip-Lock® covers. Several tubs were placed in incubation at 20 to 22° C. or 37° C.

Weekly assessment for product stability were assessed and are reported in Table 15. As noted, at all time points assessed, the color, odor, gas, visible growth and pH has not changed since Time 0.

TABLE 14
Chicken, Vegetables and Gravy Formula

| Ingredient | Amount, g |
|---|---|
| Chicken | 900 |
| Peas (canned, water drained) | 200 |
| Carrots (canned, sliced, water drained) | 200 |
| Starter Culture* | 1 |
| Gravy | 900 |

*Starter Culture contains sufficient Pediococcus acidilactici and Pediococcus pentosaceus to provide 1 × 10⁷ cfu/g of chicken and vegetables.

TABLE 15
Chicken, Vegetables and Gravy Formula Stability Results

| Time | pH | Color | Odor | Gas | Visible Growth |
|---|---|---|---|---|---|
| 0 | 4.0 | Normal | Good | None | None |
| 1 | 4.0 | Normal | Good | None | None |
| 2 | 4.0 | Normal | Good | None | None |
| 3 | 4.0 | Normal | Good | None | None |
| 4 | 4.0 | Normal | Good | None | None |
| 5 | 4.0 | Normal | Good | None | None |

### Example 17

### Use of Bacteria to Create a Gravy for a Fish and Vegetables Mixture

According to the embodiment of Example 17, Pediococci are added to preserve a gravy for use in a fish and vegetable mixture.

The gravy was prepared by the formula represented in Table 16. Chicken broth, flour, and dextrose were combined and then heated to boiling (100° C.) for one min. This mixture resulted in the formation of a gravy. This mixture was then cooled to about 49° C. and then the pediococci culture (Pediococcus acidilactici and Pediococcus pentosaceus) was added. The culture was mixed into the mixture and then allowed to incubate at 37° C. for 48 h.

Upon 48 hours incubation the gravy mixture's pH was 3.8.

TABLE 16
Gravy Formula

| Ingredient | Amount, g |
|---|---|
| Chicken Broth | 900 |
| Whole Wheat Flour | 50 |
| Dextrose | 18 |
| Starter Culture* | 1 |

*Starter Culture contains sufficient Pediococcus acidilactici and Pediococcus pentosaceus to provide 1 × 10⁷ cfu/ml of gravy.

### Example 18

### Creating a Shelf-Stable Fish and Vegetables Mixture

According to the embodiment of Example 18, a fish, vegetable, and gravy mixture was created.

Fish and vegetables were prepared by the formula represented in Table 17. Fish was placed in a Food Saver® bag and the bag was sealed. The fish in the sealed bag was then placed into an 80° C. water bath to achieve a meat cook temperature of at least 71° C. for ten min. The meat was cooked at least 74° C. for ten min. The final temperature of the meat was measured at 79° C. After cooking the meat, the food saver bag containing the meat was opened and the peas and sliced carrots were added to the meat. Starter culture (Pediococcus acidilactici and Pediococcus pentosaceus) was mixed into the meat, peas, and carrots. Finally, the gravy from Example 3 was added to the entire mixture of meat, peas, carrots and starter culture. This complete mixture was placed into Zip-Lock® tubs and sealed with Zip-Lock® covers. Several tubs were placed in incubation at 20° C. to 22° C. or 37° C.

Weekly assessments of product stability are reported in Table 18. As noted, at all time points assessed, the color, odor, gas, visible growth and pH has not changed since Time 0

TABLE 17
Fish, Vegetables and Gravy Formula

| Ingredient | Amount, g |
|---|---|
| Fish (catfish filets, boneless no skin) | 900 |
| Peas (canned, water drained) | 200 |
| Carrots (canned, sliced, water drained) | 200 |
| Starter Culture* | 1 |
| Gravy | 900 |

*Starter Culture contains sufficient Pediococcus acidilactici and Pediococcus pentosaceus to provide 1 × 10⁷ cfu/g of fish and vegetables.

TABLE 18
Fish, Vegetables and Gravy Formula Stability Results

| Time | pH | Color | Odor | Gas | Visible Growth |
|---|---|---|---|---|---|
| 0 | 4.3 | Normal | Good | None | None |
| 1 | 4.0 | Normal | Good | None | None |
| 2 | 4.0 | Normal | Good | None | None |
| 3 | 4.0 | Normal | Good | None | None |
| 4 | 4.0 | Normal | Good | None | None |
| 5 | 4.0 | Normal | Good | None | None |

### Example 19

### Bacterial Cultures Injected into Whole Meats for Human Consumption

According to the embodiment of Example 12, water is heated to about 90° F. and about 1×10⁷ CFU/g of bacterial culture (Pediococci acidilactici and Pediococci pentosaceus) and about 1% dextrose is added to create a starter culture. The bacterial solution is allowed to equilibrate for at least about 15 min before injection into a whole meat. After sufficient equilibration time, the bacterial solution is injected into meats and seafoods. The injection process involves using a needle to sufficiently place the injected solution at least about 5 mm below the surface of the meat. The choices of meats to be injected involve items such as roasts, whole chickens, hams, turkeys, and meatloafs, producing whole meat products that are protected against pathogen growth, have an extended shelf-life, reduce the need for preservatives, have improved taste and palatability, and have additional health benefits associated with the ingestion of beneficial bacteria.

### Example 20

### Chicken Meat Mixed with Squash and Inoculated with Bacteria for Product Stabilization Purposes

According to the embodiment of Example 20, a mixture of chicken meat, vegetables, and other minor ingredients are mixed, cooked, and then inoculated with a live culture of bacteria. A kettle fitted with a thermal heating jacket is capable of holding up to about 100 kg of ingredients. The stainless steel kettle is used as the basin in which the mixing and incubation of the mixture of meat, vegetables, and minor ingredients is carried out. The thermal heating jacket is capable of heating up to about 250° F. and maintaining this temperature in ambient environments as low as about −20° F. Within the kettle is a vertically oriented mixing shaft fitted with an auger for mixing. The vertical shaft is capable of rotating on the vertical axis at the rate of up to about 30 rpm. The kettle is further designed to be closed to prevent outside air from continuously contaminating the kettle's contents. The kettle maintains a one-way gas release valve to prevent internal contents from becoming pressurized and further has a thermo-coupled probe to monitor temperature as well as a probe designed to monitor the pH of the solution contained within the kettle. Contents of the kettle can be added through a port opening on top of the kettle while contents of the kettle can be removed through a port at the bottom of the kettle. About 47.8 kg of chicken meat, 45 kg of squash, 3 kg of beet pulp, 1 kg of trace minerals and vitamins, 1 kg of fish meal, 1 kg of dicalcium phosphate, 0.6 kg of potassium chloride, and 0.6 kg of salt are added into the kettle and heated to about 200° F. Upon reaching about 200° F., the mixture is cooled to about 125° F. and sufficient bacterial culture (Pediococcus acidilactici and Pediococcus pentosaceus) and dextrose are added into the mixture to provide 1×10⁷ cfu/g bacteria and 1% dextrose to create an inoculated mixture. The inoculated mixture is then added to individual trays that are then sealed. The trays containing the inoculated mixture are then incubated for about 8 hours at about 105° F. After incubation is complete, the inoculated mixture has been stabilized due to sufficient bacterial cell growth and subsequent acid production to a pH value of about 4.2. The individual trays containing the inoculated mixture are shelf stable for up to about 24 months.

### Example 21

### Bacterial Culture Spray on an Animal Carcass

According to the embodiment of Example 21, one or more chicken carcasses are sprayed with a live culture of bacteria, and subsequently, meat pieces obtained through trimming the carcasses are stabilized with the live cultures. In Example 21, water is heated to about 90° F. and about 1×10⁷ cfu/g of bacterial culture (Pediococci acidilactici and Pediococci pentosaceus) is added to create an activated starter culture. The starter culture is allowed to equilibrate for at least about 15 min before about 1% dextrose is added to create an activated, enriched starter culture. Upon adding the dextrose, the activated, enriched starter culture is sprayed onto chicken carcasses after the skin has been removed and during the slaughtering process. The activated, enriched bacterial solution is sprayed periodically throughout the slaughtering process. The surfaces of sprayed carcass components, such as necks, backs, and racks contain live and active bacterial cultures that when parts of the chicken are placed in containers for further emulsification and grinding of the meat into piece sizes smaller than 8 mm, the meat becomes inoculated throughout. Emulsified meat is then incubated at about 100° F. for at least about 4 hours, resulting in a pH value that is less than about 4.7 due to active culture growth. The meat with a pH value of less than about 4.7 is now capable of having a shelf-life up to about 3 months at storage conditions less than about 85° F.

### Example 22

### Bacterial Culture Addition to Bacon Bits Served in a Salad Bar

According to the embodiment of Example 22, a live culture of bacteria is mixed into bacon bits served at a salad bar. In Example 18, an activated, enriched starter culture is prepared in a substantially similar manner as that described above with reference to Examples 21. The starter culture is allowed to equilibrate for at least about 15 minutes before about 1% dextrose is added to create an activated, enriched starter culture. Upon adding the dextrose, the activated, enriched starter culture is mixed into bacon bits to improve stability and lessen the chance of food-borne pathogen growth and survival.

### Example 23

### Bacterial Culture Addition into Ground Beef

According to the embodiment of Example 23, a live culture of bacteria is mixed into ground beef. In Example 18, an activated, enriched starter culture is prepared in a substantially similar manner as that described above with reference to Examples 21-22. The activated, enriched starter culture is mixed into ground beef to enable stability and lessen the growth of food-borne pathogens and spoilage microorganisms. The ground beef has the unusual property of being shelf-stable in ambient storage environments and is resistant to food-borne pathogens since it has been inoculated with the activated, enriched starter culture.

### Example 24

### Bacterial Culture Addition into Ground Turkey

According to the embodiment of Example 24, a live culture of bacteria is mixed into ground turkey. In Example 24, an activated, enriched starter culture is prepared in a substantially similar manner as that described above with reference to Examples 21-23. The activated, enriched starter culture is mixed into ground turkey to enable stability and lessen the growth of food-borne pathogens and spoilage microorganisms. The ground turkey has the unusual property of being shelf-stable in ambient storage environments and is resistant to food-borne pathogens since it has been inoculated with the activated, enriched starter culture.

### Example 25

### Bacterial Culture Addition into Sushi

According to the embodiment of Example 25, a live culture of bacteria is topically applied to sushi fish meat. In Example 20, an activated, enriched starter culture is prepared in a substantially similar manner as that described above with reference to Examples 21-24. The activated, enriched bacterial culture is mixed into sushi fish meat to enable stability and lessen the growth of food-borne pathogens and spoilage microorganisms. The sushi has the unusual property of being shelf-stable in ambient storage environments and is resistant to food-borne pathogens, as it has been inoculated with the activated, enriched starter culture.

### Example 26

### Bacterial Culture Addition into Crab Cakes

According to the embodiment of Example 26, a live culture of bacteria is mixed into crab cakes. In Example 26, an activated, enriched starter culture is prepared in a substantially similar manner as that described above with reference to Examples 21-25. The activated, enriched starter culture is mixed into a crab cake meat to enable stability and lessen the growth of food-borne pathogens and spoilage microorganisms. Because of the use of mechanical mixing action of the meat and other ingredients and the continuous bathing of the meat and other ingredients, the activated, enriched starter culture permeates the crab meat and other ingredients and is active internally within the crab cake meat. The crab cake meat has the unusual property of being shelf-stable in ambient storage environments and is resistant to food-borne pathogens, as it has been inoculated with the activated, enriched starter culture.

### Example 27

### Bacterial Culture Marinating of Meat Sticks

According to the embodiment of Example 28, meat sticks are marinated with a live culture of bacteria. In Example 27, an activated, enriched starter culture is prepared in a substantially similar manner as that described above with reference to Examples 21-26. The activated, enriched starter culture is used to marinate uncooked meat sticks to enable stability and lessen the growth of food-borne pathogens and spoilage microorganisms. The marinated meat sticks are incubated at about 105° F. for about 8 hours. Because of the continuous bathing of the meat sticks, the activated, enriched starter culture permeates the meat and is active internally within the meat stick. The meat sticks have the unusual property of being shelf-stable in ambient storage environments and are resistant to food-borne pathogens, as the meat sticks have been inoculated with the activated, enriched starter culture.

### Example 28

### Bacterial Culture Marinating of Meat Strips

According to the embodiment of Example 29, meat strips are marinated with a live culture of bacteria. In Example 28, an activated, enriched starter culture is prepared in a substantially similar manner as that described above with reference to Examples 21-27. The activated, enriched starter culture is used to marinate uncooked meat strips to enable stability and lessen the growth of food-borne pathogens and spoilage microorganisms. The marinated meat strips are incubated at about 105° F. for about 8 hours. Because of the continuous bathing of the meat strips, the activated, enriched starter culture permeates the meat and is active internally within the meat stick. The meat strips have the unusual property of being shelf-stable in ambient storage environments and are resistant to food-borne pathogens, as the meat strips have been inoculated with the activated, enriched starter culture. The meat strips serve a variety of uses such as use in stir fry, snack foods, and use in cooking applications where fried or cooked items such as rice, vegetables, tomatoes, peppers, onions, or other non-meat items are also used.

### Example 29

### Bacterial Culture Marinating of Beef

According to the embodiment of Example 29, roast beef is marinated with a live culture of bacteria. In Example 26, an activated, enriched starter culture is prepared in a substantially similar manner as that described above with reference to Examples 21-28. The activated, enriched starter culture is used to marinate uncooked roast beef to enable stability and lessen the growth of food-borne pathogens. The marinated roast beef is incubated at about 105° F. for about 72 hours. Because of the continuous bathing of the roast beef, the activated, enriched starter culture permeates the meat and is active internally within the roast beef. The roast beef has the unusual property of enabling the meat to be shelf-stable in ambient storage environments and is resistant to food-borne pathogens and spoilage microorganisms, as the roast beef has been inoculated with the activated, enriched bacterial solution. The roast beef is left in the water-based carination solution to provide up to about five years of shelf-life stability. The roast beef can be served ready to eat after about 72 hours of carination in the activated, enriched starter culture without the use of cooking.

### Example 30

### Development of a Fermentation Culture

Development of a fermentation culture containing Pediococci is shown by Example 30.

Chicken broth with 2% Dextrose, 0.1% Tween 80, supplemented with 0.1% threonine; 0.05% serine; 0.05% proline; 0.1% cysteine was pasteurized to 190° F. After cooling to about 125° F., the pasteurized broth was inoculated with Pediococcus acidilactici and P. pentosaceus at the level of 1×10⁷ cfu/g. The inoculated chicken broth was placed in a sealed container and then placed in an environment at 35° C. for 48 h to allow it to ferment. After 48 hours the fermentation culture was removed from the 35° C. environment and kept at ambient temperature until the cells were removed from the fermented broth by centrifugation at 4,000 RPM for 15 min. The recovered cells were resuspended into Butterfield's Phosphate Buffer at 1×10¹ cfu/g and stored in refrigeration (4° C.).

### Example 31

### Fermentation Culture Addition to Liquid Palatants

Applying a fermentation culture to a liquid palatant in order to show the loss of bacterial activity is shown by Example 31.

The methods of growing the fermentation culture were similar to the ones used in Example 30.

To 100 g of liquid palatant, 1.5 g of fermentation culture was added. The fermentation culture was mixed into the liquid palatant by stirring with an inoculating pipette and inverting the palatant inoculum container five times. Since the fermentation cell concentrate contained about 3.2×10¹⁰ cfu/g of Pediococci, the liquid palatant contained about 4.8×10⁸ cfu/g of Pediococci. After 24 h, the level of Pediococci was assessed by the following plate method, MRS Agar at 35° C. for 48 h.

Results from the plate method indicated less than 10 cfu/g of Pediococci after 48 hours of the fermentation culture being added to the liquid palatant. These results indicate that the vast majority of Pediococci are no longer present in the palatant.

### Example 32

### Application of Liquid Palatants to Kibbles Contaminated with Salmonella Surrogates

The effect of applying a liquid palatant containing fermentation culture to kibbles contaminated with salmonella surrogate bacteria is shown by Example 32.

The method of adding the fermentation culture to the liquid palatant was done according to the method described in Example 31.

Two sources of kibbles were evaluated, one kibble source for dogs and another kibble source for cats. The kibbles for dogs contained 3.5% fat. The kibbles for cats contained 5.5% fat.

In order to simulate contaminated kibbles, surrogate organisms for salmonella were applied to the kibbles. The following E. coli strain types of ATCC (BAA 1427, BAA 1428, BAA 1429, BAA 1430, BAA 1431) were used. E. coli were applied at 1×10⁶ cfu/g to the kibbles. The result was the creation of kibbles contaminated with Salmonella and E. coli O157:H7 surrogates.

The contaminated kibble sources were divided in half. Onto the first half 1.5 g of liquid palatant was coated onto every 100 g of kibbles. Onto the second half of kibbles 1.5 g of liquid palatant containing resuspended cells as described in Example 2 was coated onto every 100 g of kibbles. The kibbles were coated by using a garden sprayer to spray the liquid palatant or liquid palatant containing the resuspended cells onto kibbles that were tumbling in a cement mixer powered by an electric motor. All coatings were done at ambient temperature. The liquid palatant containing the resuspended cells was made about 1 hours prior to it being applied to the kibbles.

Coated kibbles were then stored at 22° C. for up to four days. Daily samples of kibbles were assessed for the level of Salmonella surrogates by using Violet Red Bile Glucose (VRBG) agar to enumerate the level of E. coli present. Serial dilutions of washed kibble were used to recover surface bacteria. Serial dilutions of kibble washes were made by dilution with Butterfield's Phosphate Buffer (10⁰, 10⁻¹, 10⁻², 10⁻³). The wash was collected, pipetted into petri plates and then pour plated with VRBG agar. The plates were incubated for 24 hours at 35° C. and then counted for the number of E. coli present within a given sample.

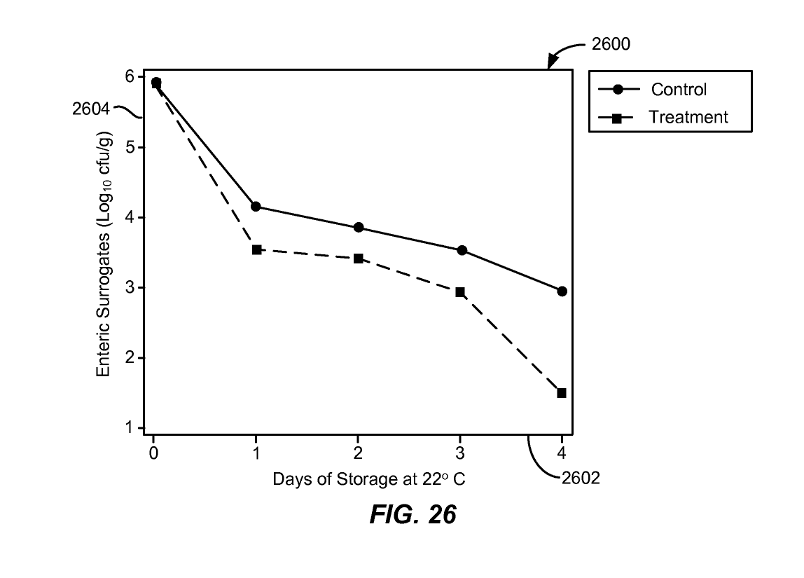

Results from cat kibbles contaminated with E. coli are shown in FIG. 25. Results from dog kibbles contaminated with E. coli are shown in FIG. 26.

FIG. 25 is line a graph 2500 showing death of Salmonella and E. coli O157:H7 surrogates on cat kibbles coated with Control (a liquid palatant alone) or Test (a liquid palatant in combination with resuspended cells). An x-axis 2502 represents the length of time (h) the control or test was applied to the kibbles. A y-axis 2504 represents the amount (log₁₀ cfu/g) of Salmonella surrogate (i.e., E. coli) growth that occurred compared to the original (Time 0) level of E. coli applied to the kibbles. The Test (dashed line with squares) indicates greater Salmonella/E. coli O157:H7 surrogate death at each time point after coating (Day 0) compared to the Control (solid line with circles).

FIG. 26 is a line graph 2600 showing death of salmonella surrogates on dog kibbles coated with Control (a liquid palatant alone) or Test (a liquid palatant in combination with resuspended cells). An x-axis 2602 represents the length of time (h) the control or test was applied to the kibbles. A y-axis 2604 represents the amount (log₁₀ cfu/g) of Salmonella/E. coli O157:H7 surrogate (i.e., E. coli) growth that occurred compared to the original (Time 0) level of E. coli applied to the kibbles. The Test (dashed line with squares) indicates greater Salmonella surrogate death at each time point after coating (Day 0) compared to the Control (solid line with circles).

These results combined with the results in Example 31, clearly indicate that the Pediococci in the fermentation culture do not need to be alive in order to kill the pathogenic bacteria over a 4 day storage period at 22° C.

### Example 33

### Impact of Liquid Palatants Containing Fermentation Culture on Food Palatability

The impact of liquid palatants containing fermentation culture applied to kibbles on food palatability is shown by Example 33.

The method of coating kibbles was done according to the method described in Example 3.

Cat and dog kibbles coated with Control (liquid palatant alone) or Test (liquid palatant in combination with fermentation culture) were compared in a preference test. The preference tests utilized 20 animals fed during a two day period. The standard split plate technique was utilized where Control was placed in one of two bowls while the Test was placed in the other bowl. Consumption of Control and Test were measured each day.

Specifically for the cat preference test, twenty cats (male and female) identified by ear tattoo and cage number were presented the test diets on an individual basis. Two stainless steel bowls, each containing approximately 100 g of diet, were offered once daily for 2 d. Bowl placement was reversed daily and both bowls were presented for 4 h. If one diet was completely consumed prior to the end of the 4 h, both bowls were removed. Food consumption and first choice preference were recorded for each cat.

Specifically for the dog preference test, twenty Beagles (male and female) identified by ear tattoo and cage number were presented the test diets on an individual basis. Two stainless steel bowls, each containing approximately 400 g of diet, were offered once daily for 2 d. Bowl placement was reversed daily and both bowls were presented for 30 min. If one diet was completely consumed prior to the end of the 30 min, both bowls were removed. Food consumption and first choice preference were recorded for each dog.

Results indicated that the total amount of food consumed by the 20 cats during the 2 d of this test was 1,352 g and was divided between the two diets as follows:

Control: 495 g or 36.6%

Test: 857 g or 63.4%

There was a 1.73 to 1 consumption ratio of Test to Control. Using a Wilcoxon Signed Rank test there was a trend to suspect a consumption difference between the two Diets (P=0.0522). Consumption ratio results for cats are shown in FIG. 27.

Results indicated that the total amount of food consumed by the 20 dogs during the 2 d of this test was 5,847 g and was divided between the two diets as follows:

Control: 2,814 g or 48.1%

Test: 3,033 g or 51.9%

There was a 1.08 to 1 consumption ratio of Test compared to Control. Using a Wilcoxon Signed Rank test there was no reason to suspect a consumption difference between the two Diets (P=0.3604). Consumption ratio results for dogs are shown in FIG. 28.

Although illustrative embodiments of the present teachings have been shown and described, other modifications, changes, and substitutions are intended. Accordingly, it is appropriate that the appended claims be construed broadly and in a manner consistent with the scope of the disclosure, as set forth in the following claims.

### Example 34

### Development of Shelf-Stable Wet Dog Food

According to the embodiment of Example 34, about 4 kg of pork hearts, 1 kg of pork liver, are added into a kettle and heated to about 180° F. for about 30 min. Then, 5 kg of sweet potato, 0.392 kg water, 0.030 g menhaden oil, 0.030 g flaxseed oil, 0.098 g sunflower oil, 0.010 g evening primrose oil, 0.246 g of trace minerals and vitamins are added into the kettle with the other ingredients and continued heated to about 180° F. for about 30 min. Next, the ingredient mixture was placed into a pot, covered and placed into an ice bath that was then placed into a freezer for about 40 min. After being in the freezer for 40 min, the pots were removed from the freezer and the temperature of the ingredient mixture was determined to be 119° F. 0.450 kg of dextrose was then added and mixed into the ingredient mixture. 10 g of Pediococcus pentosaceus and P. acidilactici was then added into the dextrose-enriched ingredient mixture that resulted in 1×10⁷ cfu/g. The product was then incubated at about 94 to about 111° F. for about 24 h. The product was then placed in individual trays (about 400 g each) and covered with a sealed lid. A sample of the product was assessed to be about pH 4.2. Product was then placed in ambient storage.

### Example 35

### Demonstration of Anti-Pathogen Attributes of Wet Pet Food

Determination of the pathogen killing effect of shelf-stable wet dog food is shown by Example 35.

In this example, a microtiter well experiment was conducted to determine the pathogen killing effect of the exudate obtained from shelf-stable wet dog food. The process to evaluate this using a microtiter plate is described as follows.

About 100 μl of chicken broth was added to wells 2 through 12 of a microtiter plate. The exudate from the shelf-stable wet dog food in Example 34 which had been stored at ambient conditions for about twelve months was then sampled. A portion of the exudate sample was used in its current formed and is herein referred to as “as is” exudate. Another portion of the exudate sample was neutralized to pH 7.0 by the addition of 0.1 N sodium hydroxide and is herein referred to as neutralized exudate. The neutralized exudate sample was used to compare the effect of non-acidified exudate to the “as is” exudate that has been acidified through the fermentation process.

About 100 μl of the “as is” exudate was added into wells 1 and 2. Well 1 served as the negative control as no growth nutrients were added (to demonstrate that no E. coli would grow in the wells without their purposeful addition to the wells). 100 μl was drawn from well 2 and placed into well 3. For well 3 and thereafter repeated 100 μl samples were drawn and placed into the next well to result in the following dilutions for wells 1 through 12: 1:1, 1:2, 1:4, 1:8, 1:16, 1:32, 1:64, 1:128, 1:256, 1:512, 1:1024, and 1:2048. The previously described procedure for wells 1 through 12 was repeated in a second set of microtiter wells using the neutralized exudate.

For both sets of microtiter wells, one set for “as is” exudate and the other set for neutralized exudate, into each well a 100 μl solution of E. coli (ATCC BAA strains 1427, 1428, 1429, 1430, 1431) was added as a pathogen source. The 100 μl solution of E. coli provided about 1×10⁷ cfu per well.

The microtiter plate was then incubated at about 35° C. for about 48 hours until all wells were dry. After the incubation time was complete and upon removal of the microtiter plate, about 100 μl of the metabolic indicator iodonitrotetrazolium chloride was added to each well. The microtiter plate was placed back into the 35° C. incubator for about 2 hours to develop the color change. Upon removal from the incubator, the results were recorded.

Results (see Table 19) indicate that the neutralized exudate was almost as effective as the “as is” exudate in killing E. coli. As such it can be concluded that the pathogen killing effect of the exudate relies on components other than simply acid to produce the E. coli killing effect.

TABLE 19
Efficacy of Either “As Is” or Neutralized Exudate Obtained from
Wet Dog Food Against E. coli Pathogens

| | | As Is Exudate | Neutralized Exudate |
|---|---|---|---|
| Well # | Dilution | (pH = 3.0) | (pH = 7.0) |
| 1 | 1:1 | − | − |
| 2 | 1:2 | − | −1 |
| 3 | 1:4 | −1 | −1 |
| 4 | 1:8 | + | + |
| 5 | 1:16 | + | + |
| 6 | 1:32 | + | + |
| 7 | 1:68 | + | + |
| 8 | 1:128 | + | + |
| 9 | 1:256 | + | + |
| 10 | 1:512 | + | + |
| 11 | 1:1024 | + | + |
| 12 | 1:2048 | + | + |
| | No Concentrate | ++ | ++ |
| | Added | | |

+ = Growth
−1 = Inhibition
− = No Growth

### Example 36

### Development of Fermented Growth Culture

Development of a fermented growth culture containing Pediococci is shown by Example 36.

2% Dextrose and 0.1% Tween 80 was added to chicken broth that was then pasteurized to about 212° F. to create a pasteurized broth mixture. When the pasteurized broth mixture had cooled to about 180° F., 0.1% threonine; 0.05% serine; 0.05% proline; 0.1% cysteine were added to create an amino acid enriched broth mixture. The amino acid enriched broth mixture was further cooled to about 125° F. Then three different inoculated broth mixtures were made by inoculating them with different culture sources. Inoculated broth mixtures included: 1) addition of 1.0 g of a freeze dried mixture of Pediococcus acidilactici and P. pentosaceus to 200 ml of inoculated broth to create a Pediococci-only broth, 2) addition of 1.0 g of freeze dried Lactobacillus plantarum to 200 ml of inoculated broth to create a Lactobacillus-only broth, and 3) addition of 0.5 g of a freeze dried mixture of Pediococcus acidilactici and P. pentosaceus and 0.5 g of freeze dried Lactobacillus plantarum to 200 ml of inoculated broth to create a Pediococci/Lactobacillus broth. Each culture was added to create an inoculated broth with a bacteria concentration of about 1×10⁷ cfu/ml.

The inoculated broth mixtures were placed in sealed containers and then placed in an environment at about 35° C. for about 48 hours to allow them to ferment to create fermented growth cultures. After 48 hours the fermented growth cultures were removed from the 35° C. environment and kept at ambient temperature until further use.

### Example 37

### Development of Live-Bacteria Concentrates

Development of three live-bacterial concentrates is shown by Example 37.

A sample of each of the three fermented growth cultures described in Example 36 were stained with crystal violet for 45 sec, then gently rinsed with water and finally visually observed using a light microscope at 1000× power in order to estimate the population density. The light microscope was used to examine the field of three random samples obtained from each fermented growth culture. Each microscope field was counted for rods and diplococci and the ratio of dipliococci to rods using a Hausner Counting Chamber. All three fermented growth cultures were confirmed to have a bacterial concentration of about 1×10⁹ cfu/ml. Further, the fermented growth culture resulting from the Pediococci/Lactobacillus broth was confirmed to have about a 1:1 ratio of Pediococci:Lactobacillus cultures.

The bacterial cells were recovered from each of the fermented growth cultures by centrifuging 200 ml of fermented growth culture at 2,500 RPM for 15 min. The supernatant was discarded and the recovered bacterial cells were resuspended in 50 ml of Butterfield's Phosphate Buffered Diluent (BPBD) at about 1×10¹⁰ cfu/g to create live bacterial cell concentrates. The live bacterial cell concentrates were concentrated four times (4λ) since 200 ml of fermented growth culture was used to obtain the recovered cells that were then reconstituted with 50 ml of BPBD (200 divided by 50 equals 4). The live bacterial cell concentrates from each of the fermented growth cultures were stored in refrigeration (4° C.).

### Example 38

### Ability of Live Bacterial Cell Concentrates to Kill Pathogens and Food-Spoilage Microorganisms

Determination of the bacterial killing ability of live bacterial cell concentrates is shown by Example 38.

In this example, a microtiter well experiment was conducted to evaluate the efficacy of the live bacterial cell concentrates as described in Example 37 on killing E. coli (ATCC BAA strains 1427, 1428, 1429, 1430, 1431). The process to evaluate this using a microtiter plate is described as follows. About 100 μl of chicken broth was added to wells 2 through 12 of a microtiter plate. About 100 μl (about 1×10⁷ cfu) of a live bacterial cell concentrate was added into wells 1 and 2.

Well 1 served as a control as no chicken broth was added. 100 μl was drawn from well 2 and placed into well 3. For well 3 and thereafter repeated 100 μl samples were drawn and placed into the next well to result in the following dilutions for wells 1 through 12: 1:1, 1:2, 1:4, 1:8, 1:16, 1:32, 1:64, 1:128, 1:256, 1:512, 1:1024, and 1:2048. The previously described procedure for wells 1 through 12 was repeated in a second and third set of microtiter wells using the other two live bacterial cell concentrates.

About 100 μl of E. coli (ATCC BAA strains 1427, 1428, 1429, 1430, 1431) solution that provided about 1×10⁷ cfu was added to wells 1 through 12 of each live bacterial cell concentrate. About 100 μal of chicken broth and about 100 μl of E. coli (ATCC BAA strains 1427, 1428, 1429, 1430, 1431) solution that provided about 1×10⁷ cfu were added into an additional microtiter plate well that served as a positive control (to demonstrate that the E. coli would grow without the addition of the live bacterial cell concentrates).

The microtiter plate was covered and placed in an incubator at about 35° C. for about 48 hours. After the appropriate incubation time the microtiter plate was removed from the incubator. About 100 μl of the metabolic indicator iodonitrotetrazolium chloride was added to each well. The microtiter plate was placed into the 35° C. incubator for about 2 hours to develop the color change. Upon removal from the incubator, the results were recorded.

Results are displayed in Table 20. Results indicate that pathogen killing power occurred with all three bacterial cell concentrates: 1) Pediococci-only broth, 2) Lactobacillus-only broth, and 3) Pediococci/Lactobacillus broth. Further, all three bacterial cell concentrates, regardless of the bacterial source had about the same killing power against E. coli. Results also indicate that the addition of Lactobacillus to a blend of Pediococci results in a compatible mixture of organisms to provide food product shelf-stability, Pediococci, and those organisms with expected health benefits, Lactobacillus.

TABLE 20
Results Comparing The Pathogen Killing Power Of Live Bacterial Cell Concentrates Obtained
From Pediococci-Only Broth, Lactobacillus-Only Broth And Pediococci/Lactobacillus Broth

| | | Live Bacterial Cell | Live Bacterial Cell | Live Bacterial Cell |
|---|---|---|---|---|
| | | Concentrate | Concentrate | Concentrate Obtained |
| | | Obtained from | Obtained from | from |
| | | Pediococci-Only | Lactobacillus-Only | Pediococci/Lactobacillus |
| Well # | Dilution | Broth | Broth | Broth |
| 1 | 1:1 | − | − | − |
| 2 | 1:2 | − | − | − |
| 3 | 1:4 | + | + | + |
| 4 | 1:8 | + | + | + |
| 5 | 1:16 | + | + | + |
| 6 | 1:32 | + | + | + |
| 7 | 1:68 | + | + | + |
| 8 | 1:128 | + | + | + |
| 9 | 1:256 | + | + | + |
| 10 | 1:512 | + | + | + |
| 11 | 1:1024 | + | + | + |
| 12 | 1:2048 | + | + | + |
| | No | ++ | ++ | ++ |
| | Concentrate | | | |

+ = Growth
−1 = Inhibition
− = No Growth

### Example 39

### Fermentation Culture Addition to a Liquid Flavor

In this example applying a fermentation culture to a liquid flavor is shown by Example 39.

The methods of growing the fermentation culture were similar to the ones used in Example 30.

To 100 g of a liquid flavor, 1.5 g of fermentation culture was added. The fermentation culture was mixed into the liquid flavor by stirring for two min. Since the fermentation culture contained about 1×10¹⁰ cfu/g of Pediococci, the liquid flavor contained about 1.5×10⁸ cfu/g of Pediococci. After 24 h, the level of Pediococci was assessed by the following plate method, MRS Agar at 35° C. for 72 hours in an anaerobic jar filled with carbon dioxide.

### Example 40

### Application of Liquid Flavor to a Ready to Eat Cereal Contaminated with Salmonella Surrogates

The effect of applying a liquid flavor containing fermentation culture to ready-to-eat cereal contaminated with salmonella surrogate bacteria is shown by Example 40.

The method of adding the fermentation culture to the liquid flavor was done according to the method described in Example 39.

A ready to eat cereal was obtained that was based on extruded corn flakes.

In order to simulate contaminated corn flakes, surrogate organisms for salmonella were applied to the corn flakes. The following E. coli strain types of ATCC (BAA 1427, BAA 1428, BAA 1429, BAA 1430, BAA 1431) were used. E. coli were applied at 1×10⁶ cfu/g to the corn flakes. The result was the creation of kibbles contaminated with Salmonella and E. coli O157:H7 surrogates.

The contaminated corn flakes source was divided in half. Onto the first half 1.5 g of liquid flavor was coated onto every 100 g of corn flakes. Onto the second half of corn flakes 1.5 g of liquid flavor containing fermentation culture as described in Example 39 was coated onto every 100 g of corn flakes. All coatings were done at ambient temperature. The liquid flavor containing the fermentation culture was made about 5 hours prior to it being applied to the corn flakes.

Coated corn flakes were then stored at 22° C. for up to four days. Daily samples of corn flakes were assessed for the level of Salmonella surrogates by using Violet Red Bile Glucose (VRBG) agar to enumerate the level of E. coli present. Serial dilutions of washed corn flakes were used to recover surface bacteria. Serial dilutions of corn flakes were made by dilution with Butterfield's Phosphate Buffer. The wash was collected, pipetted into petri plates and then pour plated with VRBG agar. The plates were incubated for 24 hours at 35° C. and then counted for the number of E. coli present within a given sample.

Results indicated greater than 1×10⁴ cfu/g loss of E. coli associated with the corn flakes coated with liquid flavor containing fermentation culture compared to the corn flakes coated with only liquid flavor.

### Example 41

### Development of a Wet Pet Food that is Shelf-Stable Free of Food Safety and Food Spoilage Organisms

Development of a wet pet food free of food safety and food spoilage organisms that is shelf-stable is shown by Example 41.

In this example, a shelf-stable wet dog food was made using the following process and formula.

All ingredients noted in Table 21 were combined and mixed together. After mixing was complete the food was incubated from about 108° F. to about 122° F. for about 20 hours to create an incubated dog food. The incubated dog food was then heated to about 190° F. and filled into individual trays (each tray containing about 400 g). Three samples of the product placed into the trays averaged pH 4.2.

The product was fed twice daily to two dogs. Within 24 hours one dog began vomiting the food and had diarrhea. Within 72 hours the other dog began vomiting the food and also had diarrhea. Although the food was determined to be shelf stable due to the low pH (4.2), the food was not well tolerated by the dogs.

TABLE 21
Wet Pet Food Ingredients

| Ingredient | Formula, % |
|---|---|
| Mechanically separated kangaroo meat | 78.456 |
| Sweet potato | 9 |
| Water | 11 |
| Dextrose | 1 |
| Salt | 0.5 |
| Live bacteria culture* | 0.044 |

*Live bacteria culture was comprised of Pediococci pentosaceus and P. acidilactici and was added at about 1 × 10⁷ cfu/g of the ingredient mixture.

### Example 42

### Development of a Wet Pet Food that Contains a Buffer

Development of a wet pet food that contains a buffer is shown by Example 42.

In this example, a buffered wet dog food was made using the following process and formula.

All ingredients noted in Table 22 were combined and mixed together. Notably, the buffer, calcium carbonate, was mixed in before the fermentation had occurred. After mixing was complete the food was incubated from about 108° F. to about 122° F. for about 20 hours to create an incubated dog food. The incubated dog food was then heated to about 180° F. and filled into individual trays (each tray containing about 400 g). Samples of the product placed into the trays averaged pH 6.17. As such, the product spoiled after 26 hours of incubation. It was concluded the buffer prevented adequate fermentation from occurring due to preventing the pH from dropping.

TABLE 22
Wet Pet Food Ingredients

| Ingredient | Formula, % |
|---|---|
| Mechanically separated kangaroo meat | 76.956 |
| Sweet potato | 9 |
| Water | 11 |
| Dextrose | 1 |
| Calcium carbonate | 2 |
| Live bacteria culture* | 0.044 |

*Live bacteria culture was comprised of Pediococci pentosaceus and P. acidilactici and was added at about 1 × 10⁷ cfu/g of the ingredient mixture.

### Example 43

### Development of Wet Pet Foods that are Shelf-Stable

Development of a wet pet food that is shelf-stable is shown by Example 43.

In this example, three shelf-stable wet dog foods, 1) low pH, 2) moderate pH/moderate temperature and 3) moderate pH/low temperature, were made using the following process and formula.

For each of the products, all ingredients noted in Table 23 except for the buffer, calcium carbonate, were first combined and mixed together. After mixing was complete, the food was incubated from about 108° F. to about 122° F. for about 18 hours to create an incubated dog food.

For the low pH product, the incubated dog food was then heated to about 180° F. and filled into individual trays (each tray containing about 400 g). Samples of the product after 18 hours of incubation averaged pH 4.4 while samples from trays after cooling to ambient temperature also averaged pH 4.4. No buffer source was added. The product was plate counted at less than 10 cfu/g of product. As such, it was concluded that the product contained no live bacteria.

For the moderate pH/moderate temperature product, calcium carbonate was added into the incubated dog food to create a buffered incubated dog food. The buffered incubated dog food was then heated to about 180° F. and filled into individual trays (each tray containing about 400 g). Samples of the product after 18 hours of incubation averaged pH 4.28 before the calcium carbonate was added while samples from trays after cooling to ambient temperature averaged pH 4.7. The product was plate counted at less than 10 cfu/g of product. As such, it was concluded that the product contained no live bacteria.

For the moderate pH/low temperature product, calcium carbonate was added into the incubated dog food to create a buffered incubated dog food. The buffered incubated dog food was then heated to about 130° F. and filled into individual trays (each tray containing about 400 g). Samples of the product after 18 hours of incubation and before the calcium carbonate was added averaged pH 4.36 while samples from trays after cooling to ambient temperature averaged pH 4.7. The product was plate counted at 8.5×10⁸ cfu/g of product. As such, it was concluded that the product contained live bacteria.

Due to the pH being decreased after incubation in each product, all products were deemed shelf-stable. The products containing the calcium carbonate were both moderated in their pH to about 4.7.

TABLE 23
Wet Pet Food Ingredients

| | | Moderate pH/ | Moderate pH/ |
|---|---|---|---|
| | | Moderate | Low |
| | Low | Temperature | Temperature |
| Ingredient | pH Product, % | Product, % | Product, % |
| Mechanically | 78.456 | 76.706 | 76.956 |
| separated kangaroo | | | |
| meat | | | |
| Sweet potato | 9 | 10 | 10 |
| Water | 11 | 10 | 10 |
| Dextrose | 1 | 2 | 2 |
| Calcium carbonate | 0 | 0.75 | 0.5 |
| Salt | 0.5 | 0.5 | 0.5 |
| Live bacteria culture* | 0.044 | 0.044 | 0.044 |

*Live bacteria culture was comprised of Pediococci pentosaceus and P. acidilactici and was added at about 1 × 10⁷ cfu/g of the ingredient mixture.

### Example 44

### Demonstration that Shelf-Stable Wet Pet Food can Provide an Acceptable Feeding Experience

Feeding studies that demonstrate an acceptable feeding experience by dogs of a shelf-stable wet dog food are shown by Example 44.

In this example, the wet dog foods made in Example 43 were fed to dogs to understand feeding acceptability and digestive tolerance of the foods.

The low pH product made in Example 43 was fed to two dogs. One dog ate the food for five days but never ate as much as was determined necessary to meet daily energy needs (average=84%). Further, consumption of the low pH product tended to decline during five days of consumption. The second dog also did not eat enough of the low pH product to meet his energy requirement (average=92%) and began having episodes of diarrhea.

The moderate pH/moderate temperature product and the moderate pH/low temperature product were compared to each other by feeding three dogs over a four day period. Each dog was offered one bowl of each food at meal times. Dogs were fed two meals per day. The first dog evaluated preferred the moderate pH/low temperature product (64% of his total four day consumption). The second dog evaluated ate both products equally. However, this dog was observed to first eat the moderate pH/low temperature product and then continued consuming the remaining allotment of the moderate pH/moderate temperature product. The third dog evaluated also preferred the moderate pH/low temperature product (57% of his total four day consumption).

Results indicated that the moderate pH/low temperature product was preferred over the moderate pH/moderate temperature product. Further, while the low pH product's pH was decreased as noted in Example 43, the product did not result in adequate consumption by the dogs as well as tended to produce episodes of diarrhea. As such, the moderate pH/low temperature product was most desirable as it was best accepted by the dogs.

### Example 45

### Taste Profile of a Wet Pet Food

A sensory study that demonstrates a more desirable taste profile of a wet dog food is shown by Example 45.

In this experiment, the wet dog foods produced by the ingredients and methods noted in Example 43 are compared for the perceived level of sourness.

Methods used in this experiment involved eight human panelists tasting the wet pet food and assessing the level of sourness of the food as a means of understanding the reasons why the pet foods resulted in the consumption differences noted in Example 44. Each subject first tasted a control product to obtain a baseline understanding of sourness. As such, the control product served as the reference product. Each subject then cleansed their palate by rinsing their mouth out with water. Next, each subject tasted the test product. Finally, each subject compared the sourness tasted on the test product to the reference product using the sensory rating scale found in Table 24.

In a first comparison test, the reference product was the low pH product as formulated and made by Example 43. The test product was the moderate pH/moderate temperature product as formulated and made by Example 43. The average score for the eight human panelists was +3.375 which represented that the test product, moderate pH/moderate temperature product, was less sour than the reference product.

In a second comparison test, the reference product was the low pH product as formulated and made by Example 43. The test product was the moderate pH/low temperature product as formulated and made by Example 43. The average score for the eight human panelists was +3.0 which represented that the test product, moderate pH/low temperature product, product was less sour than the reference product.

After both comparison tests were completed, panelists commented that the moderate pH/low temperature product was slightly more preferable because it was less tart. As such results indicate that a more ideal taste level for humans has been identified when using a moderate pH/low temperature product. These results likely explain at least in part the reasons for the moderate pH/low temperature product being more preferred by the dogs in Example 44.

TABLE 24
Sensory Rating Scale

| Rating | Description* |
|---|---|
| +4 | Extremely less sour than Reference (Control) |
| +3 | Less sour than Reference |
| +2 | Somewhat less sour than Reference |
| +1 | Slightly less sour than Reference |
| 0 | Similar to Reference |
| −1 | Slightly more sour than Reference |
| −2 | Somewhat more sour than Reference |
| −3 | More sour than Reference |
| −4 | Extremely more sour than Reference |

*Test product is compared to Control

### Example 46

### Preservation of Chicken Meat Free of Pathogens and Food Spoilage Organisms

Development of chicken meat free of food safety and food spoilage organisms is shown by Example 46.

In this example, three different whole ground chicken meat samples were made shelf-stable by adding different bacterial cultures. Into 100 g of whole ground chicken meat (pH=6.35) was mixed one of the following bacterial cultures: 1) Pediococcus acidilactici and P. pentosaceus, 2) Lactobacillus plantarum, and 3) P. acidilactici, P. pentosaceus and L. plantarum. All cultures were mixed in to provide 1×10⁷ cfu/g of chicken meat. The mixture of ground chicken meat and bacterial cultures created inoculated meat. The inoculated meat samples were then placed into an incubator set at 45° C. and incubated for 20 hours to create fermented meat samples. After 20 hours of incubation, the fermented meat samples were removed from the incubator and the pH measured. The resulting bacterial composition in the fermented meat sample created by the addition of P. acidilactici, P. pentosaceus and L. plantarum was also assessed. To do so, two different techniques were used to assess the ratio of cocci (indicative of the Pediococci strains) to rods (indicative of the Lactobacillus strain) contained within the sample. One technique involved staining the bacterial cells with crystal violet and enumerating the numbers of cocci and rods using a light microscope. Results indicated that 5.0×10⁸ cfu/g of cocci and 1.5×10⁹ cfu/g of rods were present in the fermented meat. The other technique involved using MRS agar to assess the number viable number of cocci and rods. Colony types were distinguished due to the larger colonies being rods (Lactobacillus strain) while the smaller colonies were cocci (Pediococci strains). Results indicated that 1×10⁹ cfu/g of cocci and 3×10⁹ cfu/g of rods were present in the fermented meat.

Additional results are reported in Table 25. Results noted previously using both the light microscope technique and the MRS agar confirmed similar levels of cocci and rods which indicate that the Pediococci and Lactobacillus were able to grow compatibly together. Taken together, results indicate that the ground meat sample containing the combination of P. acidilactici, P. pentosaceus and L. plantarum resulted in the lowest pH of the meat indicating a surprising and synergistic benefit of combining an organism important for intestinal health, Lactobacillus, with organisms (i.e., Pediococci) that provide shelf-stability to a food product. Additionally the food product carries the benefit of providing an organism (i.e., Lactobacillus) that is beneficial to intestinal health.

TABLE 25
Shelf Stable Food Product with Intestinal Health Benefit

| | Inoculated Ground | Ground |
|---|---|---|
| Culture Added to the Ground | Chicken Meat Length of | Chicken Meat |
| Chicken Meat | Incubation, hours | pH |
| No culture added | 0 | 6.35 |
| Pediococcus acidilactici | 20 | 4.61 |
| and P. pentosaceus | | |
| Lactobacillus plantarum | 20 | 4.53 |
| P. acidilactici, | 20 | 4.43 |
| P. pentosaceus, | | |
| and L. plantarum | | |

### Example 47

### Bacterial Culture Addition into Raw Meat Marinade

According to the embodiment of Example 24, a live culture of bacteria is mixed into a raw meat marinade. In Example 24, an activated, enriched starter culture is prepared in a substantially similar manner as that described above with reference to Examples 21-23. The activated, enriched starter culture is mixed into a marinade sauce to enable stability and lessen the chance of food borne pathogens. The marinade sauce is used to flavor raw and slightly cooked meats. Because of the use of meat mallets on the meat and the continuous bathing of the meat in the marinade the bacterial culture penetrates the meat and is active internally within the meat. The marinade has the unusual property of enabling the meat to be shelf-stable in ambient storage environments and is resistant to food borne pathogens since it has been inoculated with the activated, enriched bacterial solution.

### Example 48

### Bacterial Culture Addition into Tuna Fish Salad

According to the embodiment of Example 25, a live culture of bacteria is mixed into a tuna fish salad. In Example 25, an activated, enriched starter culture is prepared in a substantially similar manner as that described above with reference to Examples 21-24. The activated, enriched starter culture is mixed into a tuna fish salad to enable stability and lessen the growth of food-borne pathogens and spoilage microorganisms. Because of the use of mechanical mixing action of the meat and other ingredients and the continuous bathing of the meat and other ingredients, the activated, enriched starter culture permeates the tuna fish and other ingredients and is active internally within the tuna fish salad. The tuna fish salad has the unusual property of being shelf-stable in ambient storage environments and is resistant to food-borne pathogens, as it has been inoculated with the activated, enriched starter culture.

As used herein, the articles including “the”, “a” and “an” when used in a claim or in the specification, are understood to mean one or more of what is claimed or described.

As used herein, the terms “include”, “includes” and “including” are meant to be non-limiting.

As used herein, the term “animal” and “pet” means a domestic animal including, but not limited to domestic dogs, cats, horses, cows, ferrets, rabbits, pigs and the like. Domestic dogs and cats are particular examples of pets.

As used herein, the terms “animal feed”, “animal feed compositions”, “animal feed kibble”, “pet food” or “pet food composition” mean a composition intended for ingestion by a pet. Pet foods may include, without limitation, nutritionally balanced compositions suitable for daily feed, dry or semi-moist kibbles, as well as supplements or treats that may or may not be nutritionally balanced.

As used herein, the term “fines” mean a food piece less than the normal food piece size that unintentionally falls off the food piece while the food piece is in the process of being made.

As used herein, the term “starter culture,” the bacteria used in a starter culture, or the bacteria used in a topical application or a wet food composition, means a bacteria that promotes food preservation, food safety, and/or human or animal health. Such bacteria may facilitate production of a lower pH through lactic acid production, and will produce bacteriocins, hydrogen peroxide, or other metabolites, which either kill undesirable (i.e., pathogenic or food-spoilage) bacteria directly or facilitate preventing growth of undesirable bacteria.

All percentages and ratios are calculated and provided by weight unless otherwise indicated. All percentages and ratios are calculated and provided based on the total composition unless otherwise indicated.

Referenced herein may be trade names for components including various ingredients utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

Although illustrative embodiments of the present teachings have been shown and described, other modifications, changes, and substitutions are intended. Accordingly, it is appropriate that the appended claims be construed broadly and in a manner consistent with the scope of the disclosure, as set forth in the following claims.

What is claimed:

1. A topical application composition comprising:
a non-fermenting bacteria that is in a substantially non-fermenting state and is produced from fermentation of said bacteria;
a fermentation byproduct produced from said fermentation; and
a fluid portion.

2. The topical application composition of claim 1, wherein said non-fermenting bacteria and said fermentation byproduct constitute a solid residue that is present in said topical application composition in an original amount, and said topical application composition includes said solid residue in a concentrated amount that is between about 2 times and about 20 times greater than said original amount.

3. The topical application composition of claim 2, wherein said concentrated amount is about 8 times greater than said original amount.

4. The topical application composition of claim 1, wherein said fluid portion includes at least one member chosen from a group comprising water, growth medium, culture energy source, and buffered solution.

5. The topical application composition of claim 1, wherein said fluid portion is less than about 1% by weight of said topical application composition.

6. The topical application composition of claim 1, wherein an amount of said non-fermenting bacteria in said topical application is between about 1×10³ cfu/(gram of said topical application) and about 1×10¹⁰ cfu/(gram of said topical application).

7. The topical application composition of claim 1, wherein at least one of said non-fermenting bacteria includes at least one member chosen from a group comprising Pediococcus acidilactici, Pediococcus pentosaceus, Lactococcus lactis, Lactococcus cremoris, Lactobacillus delbruckii var bulgaricus, Lactobacillus plantarum, Lactobacillus pentosum, Streptococcus thermophilus, Lactobacillus sakei, Lactobacillus curvatus, Lactobacillus acidophilus, Lactobacillus reuteri, Lactobacillus plantarum, Lactobacillus casei, Lactobacillus johnsonii, Lactobacillus rhamnosus, Lactobacillus gasseri, Bifidobacterium lactis, Bifidobacterium infantis, Bifidobacterium longum, Saccharomyces boulardii, Lactobacillus salivarus, Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescents, Bifidobacterium animalis, Bifidobacterium thermophilium, Enterococcus faecalis, Streptococcus cremoris, Streptococcus salivarius, Streptococcus diacetylactis, Streptococcus intermedius, Lactobacillus paracasei, Streptococcus thermophiles, Streptococcus salivarius subsp. thermophilus, Bacillus cereus, Propionibacterium freundenreichii, Oxalobacter formagenes, Bifidobacterium bifidus, and Saccharomyces cerevisiae.

8. The topical application composition of claim 1, wherein at least one of said non-fermenting bacteria includes at least one member chosen from a group comprising Lactobacillus acidophilus, Lactobacillus reuteri, Lactobacillus plantarum, Lactobacillus casei, Lactobacillus johnsonii, Lactobacillus rahamnosus, Lactobacillus gasseri, Bifidobacterium lactis, Bifidobacterium infantis, Bifidobacterium longum, Saccharomyces boulardii, Lactobacillus salivarus, Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescsents, Bifidobacterium animalis, Bifidobacterium thermophilium, Enterococcus faecalis, Streptococcus cremoris, Streptococcus salivarius, Streptococcus diacetylactis, Streptococcus intermedius, Lactobacillus paracasei, Streptococcus thermophiles, Streptococcus salivarius subsp. Thermophilus, Bacillus cereus, Proprionibacteria freundenreichii, Bacillus coagulans (L. sporegenes), Oxalobacter formagenes, Bifidobacterium bifidus, and Saccharomyces cerevisiae.

9. The topical application composition of claim 1, wherein said fermentation byproduct includes at least one member chosen from a group comprising acid mucin, glycoprotein, bacteriocin, pediocin, hydrogen peroxide, and lactate.

10. The topical application composition of claim 1, wherein said fermentation byproduct includes at least one antimicrobial lactic acid producing bacteria metabolite chosen from a group comprising phenyllactic acid, 3-hydroxyphenyllactic acid, 4-hydroxyphenylactic acid, 3-hydroxy propanaldehyde, 1,2 propandiol, 1,3 propandiol, hydrogen peroxide, ethanol, acetic acid, carbon dioxide, carbonic acid, propanoic acid, butyric acid, cyclic dipeptides, cyclo(L-Phe-L-Pro), cyclo(L P-Traps-4-OH-L-Pro), 3-(R)-hydroxydecanoic acid, 3-hydroxy-5-cic dodecanoic acid, 3-(R)-hydroxy dodecanoic acid, and 3-(R)-hyroxytetradecanoic acid.

11. The topical application composition of claim 1, wherein said fermentation byproduct includes at least one bacteriocin that is a lantibiotic (Class II) and/or a non-lantibiotic (Class II).

12. The topical application composition of claim 1, wherein said fermentation byproduct includes at least one bacteriocin selected from a group comprising nisin A, nisin Z, nisin Q, nisin F, nisin U, nisin U2, salivarcin X, lacticin J46, lacticin 481, lacticin 3147, salivarcin A, salivarcin A2, salivarcin A3, salivarcin A4, salivarcin A5, salivarcin B, streptin, salivaricin A1, streptin, streptococcin A-FF22, BHT-Aa, BHT Ab, mutacin BNY266, mutacin 1140, mutacin K8, mutacin II, smbAB, bovicin HJ50, bovicin HC5, macedocin, plantaricin W, lactocin 5, cyctolysin, enterocin A, divercin V41, divercin M35, bavaricin, coagulin, pediocin PA-1, mundticin, piscicocin CS526, piscicocin 126/V1a, sakacin P, leucocin C, sakacin 5X, enterocin CRL35/mundticin, avicin A, mundticin I, enterocin HF, bavaricin A, ubericin A, leucocin A, mesentericin Y105, sakacin G, plantaricin 423, plantaricin C19, curvacin A/sakacin A, carnobacteriocin BM1, enterocin P, piscicoin V1b, penocin A, bacteriocin 31, bacteriocin RC714, hiracin JM79, bacteriocin T8, enterocin SE-K4, carnobacteriocin B2, SRCAM 1580, and CONCENSUS.

13. The topical application composition of claim 1, further comprising at least one member chosen from a group comprising flavor enhancer, palatant, stabilizing agent, food coating stabilizer, fragrance, binder, color, and coloring agent.

14. A substantially pathogen-free and/or spoilage-microorganism-free food composition comprising:
a topical application including:
a non-fermenting bacteria that is in a substantially non-fermenting state and is produced from fermentation of said bacteria; and
a fermentation byproduct produced from said fermentation; and
a food product having a surface that includes said topical application.

15. The substantially pathogen-free and/or spoilage-microorganism-free food composition of claim 14, wherein said food product includes less than about 10 cfu of a pathogen and/or a food-spoilage microorganism per gram of said food product.

16. A method for producing a topical application, said method comprising:
mixing a bacteria in a growth culture including a growth medium and an energy source; and
fermenting said bacteria in the presence of said growth culture to produce a fermented growth culture comprising a non-fermenting bacteria and a fermentation byproduct, and said non-fermenting bacteria is in a substantially non-fermenting state.

17. The method for producing a topical application of claim 16, wherein in said mixing, said growth culture includes a fluid portion.

18. The method for producing a topical application of claim 17, further comprising concentrating said fermented growth culture by removing a certain amount of said fluid portion from said fermented growth culture.

19. The method for producing a topical application of claim 18, wherein said concentrating includes separating an amount of said fluid portion from said fermented growth culture using at least one technique chosen from a group comprising sedimenting, centrifuging, vacuuming, decanting, drying, freeze drying, spray drying, and evaporating.

20. The method for producing a topical application of claim 16, wherein said fermenting is carried out at a temperature that is between about 28° C. and about 55° C.

21. The method for producing a topical application of claim 16, further comprising drying said fermented growth culture.

22. The method for producing a topical application of claim 16, further comprising drying said fermented growth culture.

23. A process for producing a safe and/or shelf-stable food product, said process comprising:
obtaining a topical application and a food product, and said topical application includes a non-fermenting bacteria and a fermentation byproduct, and said non-fermenting bacteria is in a substantially non-fermenting state;
applying said topical application to a surface of said food product and producing an inoculated food product; and
incubating said inoculated food product to produce a shelf-stable food product that is substantially free of pathogens and/or spoilage microorganisms.

24. The process of producing a safe and/or shelf-stable food product of claim 23, wherein said obtaining includes fermenting a bacteria to produce said non-fermenting bacteria and said fermentation byproduct.

25. The process of producing a safe and/or shelf-stable food product of claim 23, wherein said shelf-stable food product includes less than about 10 cfu of said pathogens and/or said spoilage microorganisms per gram of said shelf-stable food product.

26. The process of producing a safe and/or shelf-stable food product of claim 23, wherein said incubating is carried out at a temperature that is between about 28° C. and about 55° C.

27. The process of producing a safe and/or shelf-stable food product of claim 23, wherein said applying includes applying said topical application to said surface of said food product at a concentration that is between about 0.0001% by weight/weight of said topical application to said food product and about 10% by weight/weight of said topical application to said food product.

28. The process of producing a safe and/or shelf-stable food product of claim 23, wherein said applying is carried out using at least one technique chosen from a group comprising coating, spraying, soaking, misting, aerosolizing, affixing, and atomizing.

29. The process of producing a safe and/or shelf-stable food product of claim 23, wherein said food product includes at least one member chosen from a group comprising an ice cream, a yogurt, a milk, a meat, a fermented meat, kibbled food, kibble, expanded food, pelleted food, extruded food, refrigerated food, refrigerated treat, frozen food, frozen treat, biscuit, raw food, fried foods, treat, soft-moist food, soft-moist treat, pellet, fine, broken piece, jerky-style treat, injection-molded treat, treat, supplement, a sauce, a juice, a meal replacement drink, a probiotic drink, an OTC supplement, a prepared food, a ready-to-eat food, a functional food, a functional beverage, a whole fruit, a whole vegetable, prepared salad ingredient, ground fruit, grounded vegetable, prepared meal, meat, slaughtered carcass, prepared food, meat piece, meat chunk, fabricated meat chunk, fabricated protein chunk, livestock feed, steam-flaked feed, and aquaculture feed.

30. The process of producing a safe and/or shelf-stable food product of claim 23, wherein said food product has a moisture content of less than about 10% by weight.

31. The process of producing a safe and/or shelf-stable food product of claim 23, further comprising packaging said shelf-stable food product.

32. The process of producing a safe and/or shelf-stable food product of claim 23, wherein said non-fermenting bacteria promotes human or animal health after said shelf-stable food product is consumed by a human or an animal.

33. A process for decontaminating an inedible surface comprising applying a topical application to an inedible surface such that said topical application substantially kills and/or inhibits growth of pathogens and/or food-spoilage microorganisms on said inedible surface, and wherein said topical application includes a non-fermenting bacteria and a fermentation byproduct, and said non-fermenting bacteria is in a substantially non-fermenting state.

34. The process for decontaminating an inedible surface 33, wherein said applying is carried out using at least one technique chosen from a group comprising spraying, misting, washing, soaking, misting, aerosolizing, affixing, and atomizing.

35. The process for decontaminating an inedible surface 33, wherein said inedible surface includes at least one member chosen from a group comprising pipe, tool, chopper, grinder, hammer mill, roller mill, flaker, emulsifier, blender, block pre-breaker, block breaker, extruder, coating equipment, APEC coater, spray bar, dryer, conveyor, pellet mill, steam flaker, vortex mill, storage bin, band saw, knife, cutting surface, countertop, wood chopping block used in food preparation, stainless steel counter top, counter top, bathroom, wet bar, alcohol serving establishment, drainage system, disposal system, sink drain, kitchen sink, toilet, toilet bowl rim, bath drain, bath tub, garbage can, barn environment, barn stall, horse stall, livestock exhibition hall, livestock bedding area, retention pond, sewage holding tank, areas around sewage holding tanks, dog kennel, dog cage, cat cage, cat carrier, dog carrier, cattery, automotive garage, air recirculation system on jet airliner, shrimp shell after meat has been removed, fish parts after fillets have been removed, animal parts after meat has been removed, human hair, dog hair coat, diaper, cream, skin, dermatitis, psoriasis, eczema, bed sore, dentifrice, oral rinse, vaginal rinse, douche, tampon, feminine pad, waste pail, garbage can, dumpster, waste handling container, commercial waste management vehicle, garbage truck, waste hauling equipment, waste capture equipment, bin, can, vehicle, tote, conveyer, waste processing equipment, waste, under-arm, vagina, foot, outer ear, and diaper.

36. A wet food composition comprising:
a wet food;
a bacteria;
an energy source;
a buffer; and
wherein said wet food composition has a moisture content that is at least about 15% by weight and a pH that is between about 4.5 and about 4.9, and wherein said bacteria is a food-safety bacteria and/or a food-preserving bacteria that is substantially non-fermenting, and wherein said wet food composition is substantially free of pathogens and/or food-spoilage microorganisms.

37. The wet food composition of claim 36, wherein said buffer is at least one member chosen from a group comprising calcium carbonate, sodium bicarbonate, carbonic acid, pyrophosphates, sodium acid pyrophosphate, malic acid, potassium citrate, sodium citrate, calcium citrate, monopotassium phosphate, potassium tartrate, vinegar and tricalcium phosphate.

38. The wet food composition of claim 36, further comprising a salt and/or a syneresis-controlling substance.

39. The wet food composition of claim 38, wherein said syneresis-controlling substance is at least one member chosen from a group comprising pea powder, gum arabic, guar gum, hydrocolloid, carboxymethylcellulose, locust bean gum, cassia gum, carageenan, iota-carageenan, kappa-carageenan, milk, milk product, milk protein, casein, pork plasma, textured vegetable protein, gluten, corn gluten, wheat gluten, starch, corn starch, rice starch, potato starch, tapioca starch, sorghum starch, oat starch, soy, soy protein, soy protein concentrate, soy protein isolate, egg, egg derivatives, transglutaminase, gelatin, and polysaccharide.

40. The wet food composition of claim 38, wherein said salt is at least one member chosen from a group comprising sodium chloride, potassium chloride, sea salt, and calcium chloride.

41. The wet food composition of claim 36, further comprising a health-promoting bacteria.

42. The wet food composition of claim 36, wherein said food-safety bacteria and/or said food-preserving bacteria is at least one member chosen from a group comprising Pediococcus acidilactici, Pediococcus pentosaceus, Lactococcus lactis, Lactococcus cremoris, Lactobacillus delbruckii var bulgaricus, Lactobacillus plantarum, Lactobacillus pentosum, Streptococcus thermophilus, Lactobacillus sakei and Lactobacillus curvatus, Lactobacillus acidophilus, Lactobacillus reuteri, Lactobacillus plantarum, Lactobacillus casei, Lactobacillus johnsonii, Lactobacillus rhamnosus, Lactobacillus gasseri, Bifidobacterium lactis, Bifidobacterium infantis, Bifidobacterium longum, Saccharomyces boulardii, Saccharomyces cerevisiae, Lactobacillus salivarus, Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescents, Bifidobacterium animalis, Bifidobacterium thermophilium, Enterococcus faecalis, Streptococcus cremoris, Streptococcus salivarius, Streptococcus diacetylactis, Streptococcus intermedius, Lactobacillus paracasei, Streptococcus thermophiles, Streptococcus salivarius subsp. thermophilus, Bacillus cereus, Propionibacterium freundenreichii, and Oxalobacter formagenes.

43. The wet food composition of claim 36, wherein said health-promoting bacteria is at least one member chosen from a group comprising Lactobacillus acidophilus, Lactobacillus reuteri, Lactobacillus plantarum, Lactobacillus casei, Lactobacillus johnsonii, Lactobacillus rahamnosus, Lactobacillus gasseri, Bifidobacterium lactis, Bifidobacterium infantis, Bifidobacterium longum, Saccharomyces boulardii, Lactobacillus salivarus, Bacteroides spp, Enterococcus faecium, Lactobacillus delbrucekii spp bulgaricus, Lactobacillus cellibiosus, Lactobacillus curvatus, Lactobacillus brevis, Bifidobacterium bifidum, Bifidobacterium adolescsents, Bifidobacterium animalis, Bifidobacterium thermophilium, Enterococcus faecalis, Streptococcus cremoris, Streptococcus salivarius, Streptococcus diacetylactis, Streptococcus intermedius, Lactobacillus paracasei, Streptococcus thermophiles, Streptococcus salivarius subsp. Thermophilus, Bacillus cereus, Proprionibacteria freundenreichii, Bacillus coagulans (L. sporegenes), and Oxalobacter formagenes.

44. The wet food composition of claim 36, wherein said wet food composition has a concentration of said food-safety bacteria and/or said food-preserving bacteria that is between about 1×10³ cfu/g of said wet food composition and about 1×10⁹ cfu/g of said wet food composition.

45. The wet food composition of claim 36, wherein said wet food composition is shelf-stable for a time that is at least about six months.

46. A process for producing a wet food composition, said process comprising:
obtaining one or more food ingredients, an energy source, a food-safety bacteria and/or food-preserving bacteria, and a buffer;
mixing one or more of said food ingredients to produce a food product;
inoculating said food product with said food-safety bacteria and/or said food-preserving bacteria to produce an inoculated food product;
incubating said inoculated food product to produce an incubated food product, wherein said incubating is sufficient to produce a pH in said incubated food product that is less than about 4.3; and
adding said buffer to said incubated food product to produce said wet food composition having a pH that is between about 4.5 and about 4.9; and
wherein said wet food composition has a moisture content that is at least about 15% by weight, and said wet food composition is substantially free of pathogens and/or spoilage microorganisms.

47. The process for producing a wet food composition of claim 46, further comprising adding a salt and/or a syneresis-controlling substance to said wet food composition.

48. The process for producing a wet food composition of claim 46, wherein said inoculating includes inoculating said food product with a health-promoting bacteria.
